(19) United States
(12) Patent Application Publication
(10) Pub. No.: US 2012/0167246 A1
(43) Pub. Date: Jun. 28, 2012

(54) PLANT NUCLEIC ACIDS ASSOCIATED WITH CELLULAR pH AND USES THEREOF

(75) Inventors: Francesca Quattrocchio, Amsterdam (NL); Ronald Koes, Amsterdam (NL); Kees Spelt, Amsterdam (NL)

(73) Assignees: Stichting VU-VUmc, Amsterdam (NL); STICHTING VOOR DE TECHNISCHE WETENSCHAP, Utrecht (NL)

(21) Appl. No.: 13/318,315

(22) PCT Filed: Apr. 30, 2010
(86) PCT No.: PCT/AU10/00504

§ 371 Date: Jan. 13, 2012

(30) Foreign Application Priority Data
May 1, 2009 (AU) 2009901920

(51) Int. Cl. C12N 5/10; C12N 15/82; A01H 1/06; A01H 5/00; A01H 5/10; A01H 5/08; A01H 5/06; A01H 5/04; A01H 5/12; C12N 15/29; A01H 5/02

(52) U.S. Cl. 800/278; 536/23.6; 435/320.1; 435/419; 800/298

(57) ABSTRACT

The present invention relates generally to the field of plant molecular biology and agents useful in the manipulation of plant physiological and biochemical properties. More particularly, the present invention provides genetic and proteinaceous agents capable of modulating or altering the level of acidity or alkalinity in a cell, group of cells, organelle, part or reproductive portion of a plant. Genetically altered plants, plant parts, progeny, subsequent generations and reproductive material including flowers or flowering parts having cells exhibiting an altered cellular including vacuolar pH compared to a non-genetically altered plant are also provided.

This application is associated with and claims priority from Australian Provisional Patent Application No. 2009901920, filed on 1 May, 2009, entitled “Nucleic acid molecules and uses therefor”, the entire contents of which, are incorporated herein by reference.

### FIELD

The present invention relates generally to the field of plant molecular biology and agents useful in the manipulation of plant physiological and biochemical properties. More particularly, the present invention provides genetic and proteinaceous agents capable of modulating or altering the level of acidity or alkalinity in a cell, group of cells, organelle, part or reproductive portion of a plant. Genetically altered plants, plant parts, progeny, subsequent generations and reproductive material including flowers or flowering parts having cells exhibiting an altered cellular including vacuolar pH compared to a non-genetically altered plant are also provided.

### BACKGROUND

Reference to any prior art in this specification is not, and should not be taken as, an acknowledgment or any form of suggestion that this prior art forms part of the common general knowledge in any country.

Bibliographic details of references provided in the subject specification are listed at the end of the specification.

The cut-flower, ornamental and agricultural plant industries strive to develop new and different varieties of plants with features such as novel flower colors, better taste/flavor of fruits (e.g. grapes, apples, lemons, oranges) and berries (e.g. strawberries, blueberries), improved yield, longer life, increased nutritional content, novel colored seeds for use as proprietary tags, tolerance to abiotic factors and accumulation of specific molecules.

Furthermore, plant byproduct industries which utilize plant parts value novel products which have the potential to impart altered characteristics to their products (e.g. juices, wine) such as, appearance, style, taste, smell and texture.

In the cut flower and ornamental plant industries, an effective way to create such novel varieties is through the manipulation of flower color. Classical breeding techniques have been used with some success to produce a wide range of colors for almost all of the commercial varieties of flowers and/or plants available today. This approach has been limited, however, by the constraints of a particular species' gene pool and for this reason it is rare for a single species to have the full spectrum of colored varieties. For example, the development of novel colored varieties of plants or plant parts such as flowers, foliage and stems would offer a significant opportunity in both the cut flower and ornamental markets. In the cut flower or ornamental plant industry, the development of novel colored varieties of major flowering species such as rose, chrysanthemum, tulip, lily, carnation, gerbera, orchid, lisianthus, begonia, torenia, geranium, petunia, nierembergia, pelargonium, iris, impatiens and cyclamen would be of great interest. A more specific example would be the development of a blue rose for the cut flower market.

To date, creation of a “true” blue shade in cut flowers has proven to be extremely difficult. Success in creating colors in the “blue” range has provided a series of purple colored carnation flowers (see the website for Florigene Pty Ltd, Melbourne, Australia; and International Patent Application PCT/AU96/00296). These are now on the market in several countries around the world. There is a need, however, to generate altered flower colors in other species in addition to bluer colors in carnation and other cut flower species such as Rosa spp, Dianthus spp, Gerbera spp, Chrysanthemum spp, Dendranthema spp, lily, Gypsophila spp, Torenia spp, Petunia spp, orchid, Cymbidium spp, Dendrobium spp, Phalaenopsis spp, Cyclamen spp, Begonia spp, Iris spp, Alstroemeria spp, Anthurium spp, Catharanthus spp, Dracaena spp, Erica spp, Ficus spp, Freesia spp, Fuchsia spp, Geranium spp, Gladiolus spp, Helianthus spp, Hyacinth spp, Hypericum spp, Impatiens spp, Iris spp, Chamelaucium spp, Kalanchoe spp, Lisianthus spp, Lobelia spp, Narcissus spp, Nierembergia spp, Ornithoglaum spp, Osteospermum spp, Paeonia spp, Pelargonium spp, Plumbago spp, Primrose spp, Ruscus spp, Saintpaulia spp, Solidago spp, Spathiphyllum spp, Tulip spp, Verbena spp, Viola spp, Zantedeschia spp, etc. It is apparent that other plants have been recalcitrant to genetic manipulation of flower color due to certain physiological characteristics of the cells.

One such physiological characteristic is vacuolar pH.

In all living cells, the pH of the cytoplasm is about neutral, whereas in the vacuoles and lysosomes an acidic environment is maintained. The H⁺-gradient across the vacuolar membrane is a driving force that enables various antiporters and symporters to transport compounds across the vacuolar membrane. The acidification of the vacuolar lumen is an active process. Physiological work indicated that two proton pumps, a vacuolar H⁺ pumping ATPase (vATPase) and a vacuolar pyrophosphatase (V-PPase), are involved in vacuolar acidification.

Vacuoles have many different functions and different types of vacuoles may perform these different functions.

The existence of different vacuoles also opens complementary questions about vacuole generation and control of the vacuolar content. The studies devoted to finding an answer to this question are complicated by the fact that isolation and evacuolation of cells (protoplast isolation and culture) induces stress that results in changes in the nature of the vacuolar environment and content.

Mutants in which the process of vacuolar genesis and/or the control of the internal vacuolar environment are affected are highly valuable to allow the study of these phenomena in intact cells in the original tissue. Mutants of this type are not well described in the literature. This has hampered research in this area.

Flower color is predominantly due to three types of pigment:flavonoids, carotenoids and betalains. Of the three, the flavonoids are the most common and contribute to a range of colors from yellow to red to blue. The flavonoid pigments are secondary metabolites of the phenylpropanoid pathway. The biosynthetic pathway for the flavonoid pigments (flavonoid pathway) is well established (Holton and Cornish, Plant Cell 7:1071-1083, 1995; Mol et al, Trends Plant Sci. 3: 212-217, 1998; Winkel-Shirley, Plant Physiol. 126:485-493, 2001a; Winkel-Shirley, Plant Physiol. 127:1399-1404, 2001b, Tanaka et al, Plant Cell, Tissue and Organ Culture 80 (1):1-24, 2005, Koes et al, Trends in Plant Science, May 2005).

The flavonoid molecules that make the major contribution to flower or fruit color are the anthocyanins, which are glycosylated derivatives of anthocyanidins. Anthocyanins are generally localized in the vacuole of the epidermal cells of petals or fruits or the vacuole of the sub epidermal cells of leaves. Anthocyanins can be further modified through the addition of glycosyl groups, acyl groups and methyl groups. The final visible color of a flower or fruit is generally a combination of a number of factors including the type of anthocyanin accumulating, modifications to the anthocyanidin molecule, co-pigmentation with other flavonoids such as flavonols and flavones, complexation with metal ions and the pH of the vacuole.

The vacuolar pH is a factor in anthocyanin stability and color. Although a neutral to alkaline pH generally yields bluer anthocyanidin colors, these molecules are less stable at this pH.

Vacuoles occupy a large part of the plant cell volume and play a crucial role in the maintenance of cell homeostasis. In mature cells, these organelles can approach 90% of the total cell volume, can store a large variety of molecules (ions, organic acids, sugar, enzymes, storage proteins and different types of secondary metabolites) and serve as reservoirs of protons and other metabolically important ions. Different transporters on the membrane of the vacuoles regulate the accumulation of solutes in this compartment and drive the accumulation of water producing the turgor of the cell. These structurally simple organelles play a wide range of essential roles in the life of a plant and this requires their internal environment to be tightly regulated.

There is a need to be able to manipulate the pH in plant cells and organelles in order to generate desired flower colors and other altered characteristics such as taste and flavor in tissues such as fruit including berries and other reproductive material.

### SUMMARY

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Nucleotide and amino acid sequences are referred to by a sequence identifier number (SEQ ID NO:). The SEQ ID NOs: correspond numerically to the sequence identifiers <400>1 (SEQ ID NO:1), <400>2 (SEQ ID NO:2), etc. A summary of the sequence identifiers is provided in Table 1. A sequence listing is provided after the claims.

The present invention provides a nucleic acid molecule derived, obtainable or from plants encoding a polypeptide having pH modulating or altering activity and to the use of the nucleic acid molecule and/or corresponding polypeptide to generate genetic agents or constructs or other molecules which manipulate the pH in a cell, groups of cells, organelles, parts or reproductions of a plant. The nucleic acid molecule is referred to herein as “PH1”. Reference to “PH1” includes its homologs, orthologs, paralogs, polymorphic variants and derivatives from a range of plants. Particular PH1 genes and gene products are from rose, petunia, grape and carnation.

Manipulation of vacuolar pH is a particular embodiment herein including modulating levels of PH1 or PH1 in combination with PH5. The PH5 gene is disclosed in Verweij et al, Nature Cell Biology 10:1456-1462, 2008 and in International Patent Application Nos. PCT/AU2006/000451 and PCT/AU2007/000739, the entire contents of which are incorporated by reference. Controlling the pH pathway, and optionally, together with manipulation of the anthocyanin pathway and/or an ion transport pathway provides a powerful technique to generate altered colors or other traits such as taste or flavor, especially in rose, carnation, gerbera, chrysanthemum, lily, gypsophila, apple, begonia, Euphorbia, pansy, Nierembergia, lisianthus, grapevine, Kalanchoe, pelargonium, Impatiens, Catharanthus, cyclamen, Torenia, orchids, Petunia, iris, Fuchsia, lemons, oranges, grapes and berries (such as strawberries, blueberries). Reference to alteration of the anthocyanin pathway includes modulating levels of inter alia flavonoid 3′,5′ hydroxylase (“F3′5′H”), flavonoid 3′ hydroxylase (“F3′H”), dihydroflavonol-4-reductase (“DFR”) and methyltransferases (MT) which act on anthocyanin.

Accordingly, genetic agents and proteinaceous agents are provided which increase or decrease the level of acidity or alkalinity in a plant cell. The ability to alter pH enables manipulation of flower color. The agents include nucleic acid molecules such as cDNA and genomic DNA or parts or fragments thereof, antisense, sense or RNAi molecules or complexes comprising same, ribozymes, peptides and proteins. In a particular embodiment, the vacuolar pH is altered by manipulation of PH1. As indicated above, PH1 may be manipulated alone or in combination with other pH altering genes or proteins such as PH5. Furthermore, PH1 (and optionally PH5) may be manipulated in combination with an ion pump such as a sodium-potassium antiporter or other cation-proton antiporter transporter for the purposes of altering flower color and other infloresence and/or taste or flavor of fruit including berries and other reproductive material.

In particular, the present invention provides, in one embodiment, a method for increasing pH to make a cell or vacuole or other compartment more alkaline by decreasing the level of PH1 protein or activity. Plants comprising such cells produce flowers with a blue to purple color. In another embodiment, a method is provided for decreasing pH to make a cell or vacuole or other compartment more acidic by increasing the level of PH1 protein or activity. Plants comprising such cells produce flowers with a red to crimson color. Altered cell or organelle (e.g. vacuolar) pH can also lead to an altered taste or flavor such as in fruit including berries and other reproductive material.

Another aspect relates to a nucleic acid molecule comprising a sequence of nucleotides encoding or complementary to a sequence encoding a protein which exhibits a direct or indirect effect on cellular pH, and in particular vacuolar pH. In one embodiment, the nucleic acid is PH1 from a plant such as but not limited to rose, petunia, grape and carnation. The nucleic acid molecule may be a cDNA or genomic molecule.

Levels of expression of the subject PH1 nucleic acid molecule to be manipulated or to be introduced into a plant cell alter cellular pH, and in particular vacuolar pH. This in turn permits flower color or taste or other characteristics to be manipulated.

In particular, decreasing levels of activity of PH1 alone or in combination with PH5 leads to an increase in pH to alkaline conditions. Increasing levels or activity of PH1 alone or in combination with PH5 leads to a decrease in pH to acidic conditions.

Genetically modified plants are provided exhibiting altered flower color or taste or other characteristics. Reference to “genetically modified” plants includes the first generation plant or plantlet as well as vegetative propagants and progeny and subsequent generations of the plant. Reference to a “plant” includes reference to plant parts including reproductive portions, seeds, flowers, stems, leaves, stalks, pollen and germplasm, callus including immature and mature callus.

A particular aspect described herein relates to down regulation of PH1 which increases the level of alkalinity, leading to an increase in cellular, and in particular vacuolar, pH in a plant, resulting in bluer colored flowers in the plant. In another particular aspect, elevated regulation of PH1 which increases the level of acidity, leading to a decrease in cellular, and in particular vacuolar pH, resulting in redder colored flowers in a plant. This may require additional manipulation of levels of indigenous or heterologous PH5, F3′5′H, F3′H, DFR and MT enzymes. Altered pH levels can also lead to changes in taste and flavor in various tissues such as fruit including berries and other reproductive material.

The present invention provides, therefore, a PH1 or PH1 homolog from a plant which:
(i) comprises a nucleotide sequence which has at least 50% identity to SEQ ID NOs:1, 3, 42, 44, 58 or 59 after optimal alignment;
(ii) comprises a nucleotide sequence which is capable of hybridizing to SEQ ID NOs:1, 3, 42, 44, 58 or 59 or its complement;
(iii) encodes an amino acid sequence which has at least 50% similarity to SEQ ID NOs:2, 4, 43 or 45 after optimal alignment; and
(iv) when expressed in a plant cell or organelle, leads to acidic conditions or when its expression is reduced in a plant cell or organelle, leads to alkaline conditions.

In an embodiment, the PH1 or its homolog is capable of complementing a PH1 mutant in the same species from which it is derived. In a particular embodiment, the PH1 can complement a ph1 mutant in petunia.

The present invention further contemplates the use of a PH1 or its homolog as defined above in the manufacture of a transgenic plant or genetically modified progeny thereof exhibiting altered inflorescence or other characteristics such as taste or flavor such as in fruit including berries and other reproductive material.

Cut flowers are also provided including severed stems containing flowers of the genetically altered plants or their progeny in isolated form or packaged for sale or arranged on display.

The nucleic acid molecule and polypeptide encoded thereby corresponding to PH1 is particularly contemplated herein. Genetically modified plants having an altered PH1 alone or in combination with PH5 and the expression (or reduction in expression) of anthocyanin modifying genes such as F3′5′H, F3′H, DFR and MT as well as ion transporters such as a sodium-potassium antiporter are encompassed by the present invention for the purposes of altering flower color and other infloresence and/or taste or flavor of fruit including berries and other reproductive material.

TABLE 1
Summary of sequence identifiers

| SEQ | | | |
|---|---|---|---|
| ID | | Type of | |
| NO: | Sequence name | sequence | Description |
| 1 | RosePH1 cDNA | Nucleotide | cDNA nucleotide |
| | | | sequence of Rosa |
| | | | hybrida PH1 |
| 2 | RosePH1 protein | Amino acid | Deduced amino acid |
| | (deduced sequence) | | sequence of Rosa |
| | | | hybrida PH1 |
| 3 | PetuniaPH1 cDNA | Nucleotide | cDNA nucleotide |
| | | | sequence of Petunia |
| | | | hybrida PH1 |
| 4 | petuniaPH1 protein | Amino acid | Deduced amino acid |
| | | | sequence of Petunia |
| | | | hybrida PH1 |
| 5 | PH1 Rose/MS fw1 | Nucleotide | Primer |
| 6 | PH1 Rose/MS rev1 | Nucleotide | Primer |
| 7 | PH1 Rose/MS fw2 | Nucleotide | Primer |
| 8 | PH1 Rose/MS rev2 | Nucleotide | Primer |
| 9 | PH1 Rose/MS fw3 | Nucleotide | Primer |
| 10 | PH1 Rose/MS rev3 | Nucleotide | Primer |
| 11 | PH1 deg. bp4520 F | Nucleotide | Primer |
| 12 | PH1 deg. bp3355 F | Nucleotide | Primer |
| 13 | PH1 deg. bp6405 F | Nucleotide | Primer |
| 14 | PH1 deg. bp6650 R | Nucleotide | Primer |
| 15 | PH1 deg bp7150 R | Nucleotide | Primer |
| 16 | PH1 deg bp4463 F | Nucleotide | Primer |
| 17 | PH1 deg bp4463 R | Nucleotide | Primer |
| 18 | PH1 deg bp6410 R | Nucleotide | Primer |
| 19 | PH1 deg. 560 F | Nucleotide | Primer |
| 20 | PH1 deg. 580 R | Nucleotide | Primer |
| 21 | PH1 deg. 630 R | Nucleotide | Primer |
| 22 | PH1 deg. bp1440 F | Nucleotide | Primer |
| 23 | PH1 deg. bp2300 R | Nucleotide | Primer |
| 24 | PH1Rose bp187(cds) F | Nucleotide | Primer |
| 25 | PH1 Rose bp2030(cds) R | Nucleotide | Primer |
| 26 | PH1 Rose bp3040 F | Nucleotide | Primer |
| 27 | PH1 Rose bp1222 R | Nucleotide | Primer |
| 28 | PH1 Rose bp1170 R | Nucleotide | Primer |
| 29 | PH1 Rose bp1460 F | Nucleotide | Primer |
| 30 | PH1 Rose bp2540 F | Nucleotide | Primer |
| 31 | PH1 Rose bp720 R | Nucleotide | Primer |
| 32 | PH1 Rose bp740 R | Nucleotide | Primer |
| 33 | PH1 Rose bp720 F | Nucleotide | Primer |
| 34 | PH1 Rose Stop R | Nucleotide | Primer |
| 35 | PH1 Rose ATG Topo F | Nucleotide | Primer |
| 36 | PH1 Rose bp240 R | Nucleotide | Primer |
| 37 | PH1 Rose bp330 F | Nucleotide | Primer |
| 38 | PH1 Rose bp900 R | Nucleotide | Primer |
| 39 | PH1 Rose bp1680 R | Nucleotide | Primer |
| 40 | PH1 Rose ATG + attB1 F | Nucleotide | Primer |
| 41 | PH1 Rose stop + attB2 R | Nucleotide | Primer |
| 42 | PH1 Grape cv Pinot Noir | Nucleotide | Nucleotide sequence |
| | | | of Vitis vinifera cv |
| | | | Pinot Noir |
| 43 | PH1 Grape cv Pinot Noir | Amino acid | Amino acid sequence |
| | | | of Vitis vinifera cv |
| | | | Pinot Noir |
| 44 | PH1 Grape cv Nebbiolo | Nucleotide | Nucleotide sequence |
| | | | of Vitis vinifera cv |
| | | | Nebbiolo |
| 45 | PH1 Grape cv Nebbiolo | Amino acid | Amino acid of PH1 |
| | | | Vitis vinifera cv |
| | | | Nebbiolo |
| 46 | PH5 Phusion PCR | 2438 Primer | Primer |
| 47 | PH5 Phusion PCR | 2078 Primer | Primer |
| 48 | PH1 Grape cv Nebbiolo | 4836 Primer | Primer |
| 49 | PH1 Grape cv Nebbiolo | 4934 Primer | Primer |
| 50 | PH1 Grape cv Nebbiolo | 4933 Primer | Primer |
| 51 | PH1 Grape cv Nebbiolo | 4936 Primer | Primer |
| 52 | PH1 Grape cv Nebbiolo | 4935 Primer | Primer |
| 53 | PH1 Grape cv Nebbiolo | 4837 Primer | Primer |
| 54 | RosePH1 | 4446 Primer | Primer |
| 55 | RosePH1 | 4447 Primer | Primer |
| 56 | PH1 Phusion polymerase | 4001 Primer | Primer |
| 57 | PH1 Phusion polymerase | 3917 Primer | Primer |
| 58 | Petunia PH1 genomic | Nucleotide | Genomic nucleotide |
| | | | sequence of Petunia |
| | | | hybrida PH1 |
| 59 | Grape cv Pinot Noir | Nucleotide | Genomic nucleotide |
| | PH1.genomic | | sequence of PH1 from |
| | | | Vitis vinifera cv |
| | | | Pinot Noir |

Refence to “Rose” means Rosa hybrida.
Refernece to “Grape” means a cultivar of Vitis vinifera.

### BRIEF DESCRIPTION OF THE FIGURES

Some figures contain color representations or entities. Color photographs are available from the Patentee upon request or from an appropriate Patent Office. A fee may be imposed if obtained from a Patent Office.

FIG. 1 is a photographic, diagrammatic and schematic representation of the cloning and characterization of the PH1 gene. A) the stable ph1 mutant line R67 was crossed to the an1 unstable line W138. In the F1 progeny, plant L2164-1 showed a ph mutant phenotype. B) Scheme of the PH1 gene, with the position of the transposon insertion in the allele ph1^{L2164} and that of the mutation in the stable mutant line R67 and V23 indicated. C) Phylogenetic relation among known magnesium translocating P-type ATPases. No similar proteins have been found in animals. In fungi these proteins are represented in Ascomycetes, however baker's yeast does not have members of this family. In plants, only a few families are known to have these pumps, Arabidopsis does not. The tree is constructed by pairwise alignment between the PH1 protein sequence and the non redundant protein database (see D). D) Sequence analysis of mutant and revertant alleles of PH1:-WT sequence of the WT PH1 allele, L2164-1 sequence of the mutant allele isolated in the tagging experiment. In this allele a dTPH1 copy is inserted in the coding sequence of CAC7.5 (13 by after the ATG of the predicted protein sequence) and gave rise to a target site duplication of 8 bp, M1016-2 and M1017-1 are two revertant plants that harbor wild-type red flowers. The PH1 alleles in these plants originated from two independent excision events of dTPH1 in backcross progeny of L2164-1. In both cases a 6 by footprint was created at the site of insertion of the transposon. In the second group of sequences, stable PH1 mutant alleles are analyzed. WT: sequence of the PH1 gene, R67/V23 sequence found at the same site in the PH1 alleles of the stable mutant lines R67 and V23 (8 by insertion), the lines V42 and V48 show a 7 by insertion at the same site.

FIG. 2 is a diagrammatic representation of a comparison of members of the p-ATPases superfamily. The tree was constructed from sequences of proteins belonging to the IIIA group (of which PH5 is member) and IIIB group (of which PH1 is member). For comparison, a member of the IIA group is also included.

FIG. 3 is a photographic and graphical representation of the effect of PH1 and PH5 on petal coloration and vacuolar lumen acidification. A) effect of the ph1 mutation on the phenotype of petunia flowers accumulating different anthocyanins. 1: WT (malvidin); 2:rt, hf1 ph1 (cyanidin); 3:rt, Hf1, ph1 (delphinidin); 4:Fl, ph1^{m} (malvidin combined with flavonols); 5:fl, ph1^{m} (malvidin and no flavonols). B) pH value of the crude extracts of petals and leaves in wild-type versus ph mutant plants and in transgenics ectopically expressing PH1, PH5 or the combination of the two. While neither PH1 nor PH5 alone can complement the regulatory mutant ph3, or acidify leaf tissue, the combined expression of the two fully complements the ph3 mutant and strongly acidifies the vacuoles of leaves. Reddish bars indicate flowers with WT phenotype, bluish bars flowers with ph mutant phenotype and green bars, leaf extracts. C) Phenotypes of the plants used in the experiment shown in panel B.

FIG. 4 is a diagrammatic representation of the model explaining the involvement of PH5 and PH1 in modifying the pH of the vacuolar lumen. A) PH5 pumps protons into the vacuole using energy provided by ATP. When the electrochemical potential across the tonoplast becomes high, PH5 cannot pump anymore protons across the membrane, until Mg²⁺ cations are removed by the activity of PH1. If PH1 is absent, the proton pumping activity of PH5 is limited and the vacuolar lumen remains relatively alkaline, which prevents the generation of blue pigment. B) The characterized function of PH5 is to establish a proton gradient, which is used by a MATE protein allowing for the accumulation of proanthocyanin molecules inside the vacuole. With the evolution of flowering higher plants and the need to attract pollinators for reproduction, it was thought that the activity of PH5 was also directed towards keeping the pH of the vacuolar lumen low. This would allow for coloration of flower petals which is important for attracting pollinators. On the tonoplast of these cells is an ATP-dependent MPR-like transporter, the activity of which allows for the accumulation of anthocyanins in the vacuole. The activity of PH5 generates an electrochemical gradient, as well as a proton gradient, which is regulated by the cation pumping activity of PH1.

FIG. 5 (1026 PH1 rose gDNA-pEnt) is a diagrammatic representation of the genomic PCR fragment containing the complete coding sequence (from ATG to STOP codon) of PH1 from rose, cloned between the recombination sites of the Gateway Entry vector PEnt.

FIG. 6 (1027 35S:PH1 rose gDNA in pK2GW7) is a diagrammatic representation of the rose PH1 genomic fragment derived from the construct in described in FIG. 5 following cloning into the expression vector pK2GW7 between the 35S promoter and the 35S terminator. This construct confers resistance to Kanamicin in plant cells.

FIG. 7 (1028 35S:PH1 rose gDNA in pB7WG2.0) is a diagrammatic representation of the rose PH1 genomic fragment derived from the construct in described in FIG. 5 following cloning into the expression vector pB7GW2.0 between the 35S promoter and the 35S terminator. This construct confers resistance to the herbicide Basta in plant cells.

FIG. 8a is a diagrammatic representation of construct 1020. Petunia PH1 genomic fragment in entry vector (Pentr/d-top( )). From this it was recombined into vector V178 (pB7WG2,0) to give the expression construct 1025 (FIG. 8b).

FIG. 8b is a diagrammatic representation of construct CaMV 35 promoter: Petunia hybrida (Ph)PH1 genomic fragment:T35S terminator in vector V178 (pB7WG2,0).

FIG. 8c is a diagrammatic representation of clone 831. gDNA fragment of Petunia hybrida PH5 in pEZ-LC.

FIG. 8d is a diagrammatic representation of clone 835. Genomic fragment of Petunia hybdrida PH5 plus OCS terminator in pENTR4.

FIG. 8e is a diagrammatic representation of construct 0836 (893) for expression of Petunia hybrida PH5 in plants containing 35S: petunia PH5:35 S expression cassette in a binary transformation vector.

FIG. 9 is a graphical representation of pH values measured in crude extracts of flowers with pH mutant phenotype (blue bars), pH wild-type phenotype (red bars) and leaves (green bars).

FIG. 10a is a diagrammatic representation of construct 1218 containing grape PH1 sequence. Insert obtained by tailoring two grape cDNA fragments and one grape gDNA fragment to introduce one intron. Fragment C1+G1+C3. Complete fragment of 3.5 kb in V194=clone 1215 (FIG. 10c). This clone obtained by LR reaction of clone 1215×pK2GW7,0(V137). Heterozygous allele gives one mutation in aa299 N>Y.

FIG. 10b is a diagrammatic representation of construct 1219 containing grape PH1 sequence. Insert obtained by tailoring two grape cDNA fragments and one grape gDNA fragment to introduce two introns. Fragment C1+G2+C4. Complete fragment of 3.8 kb in V194=clone 1216 (FIG. 10d). This clone obtained by LR reaction of clone 1216×pK2GW7.0(V137). Heterozygous allele gives two mutations in aa38A>T and aa113 H>R.

FIG. 10c is a diagrammatic representation of construct 1215 containing grape PH1 sequence. Insert obtained by tailoring two grape cDNA fragments and one grape gDNA fragment to introduce one intron. Fragment C1:PCR on cDNA with primers 4836(+attB1) and 4934=>800 bp. Fragment G1:PCR on gDNA with primers 4933 and 4938=>1000 bp. Fragment C3:PCR on cDNA with primers 4937 and 4837(+attB2)=>2000 bp. Complete fragment of 3.5 kb recombined with pDONR221 by BP reaction. Heterozygous allele gives one mutation in aa299 N>Y.

FIG. 10d is a diagrammatic representation of construct 1216 containing grape PH1 sequence. Insert obtained by tailoring two grape cDNA fragments and one grape gDNA fragment to introduce two introns. Fragment C1:PCT on cDNA with primers 4836(+attB1) and 4934=>800 bp. Fragment G2:PCR on gDNA with primers 4933 and 4936=>1900 bp. Fragment C4:PCR on cDNA with primers 4935 and 4837(+attB2)=>1400 bp. Complete fragment of 3.8 kb recombined with pDONR221 by BP reaction. Heterozygous allele gives two mutations in aa38A>T and aal 13 H.R.

FIG. 10e is a diagrammatical representation of construct 1027 for expression of rose PH1. Obtained by LR reaction from gDNA_pENTR (clone 1026)×pK2GW7,0(V137). The LR reaction means entry clone+destination vector=expression clone. See website for Gateway cloning (Invitrogen).

FIG. 10f is a diagrammatical representation of clone 1026. Phusion PCR fragment; primers 4446+4447; BP reaction with pDONR207. The BP reaction means PCR fragment+donor clone=entry clone. See website for Gateway cloning (Invitrogen).

FIGS. 11a through c are photographic representations of complementation of the ph1 mutant phenotype in petunia with the 35S:Petunia hybrida (Ph)PH/gDNA-GFP. The mutant hybrid in which the transgenics where generated is M1015 ph1⁻ (R170×V23). An untransformed control shown on the left, a complementant on the right. FIG. 11b shows complementation of the petunia ph1 mutant hybrid M1020 ([V23XV30]XS) with the 35S:PH1 rose gDNA. On the left a flower from a complemented plant (P7022-1) on the right an untransformed M1020 control. FIG. 11c shows the complementation of the petunia ph1 mutant hybrid M1020 ([V23XV30]XS) with the 35S:PH1 grape gene. The flower in the picture comes from a plant complemented with construct 1218, the phenotype of plants complemented with construct 1229 is just identical. On the right the complemented flower (from plant P7079-2) and on the right an untransformde M1020 ph1 mutant. The M1020 hybrid is a selfing of the original heterozygous wild-type V23XV30. This results in a segragating population of wild-type heterozygous plants (with red flowers and low pH of the crude petal extract) and mutant homozygous plants (with blue flowers and high pH of the crude petal extract). Homozygous mutant plants where chosen as host for transformation.

FIG. 12 is a diagrammatic representation of a phylogenetic tree obtained alligning the fullsize protein sequence of PH1 homologs from the bacteria Bacillus cereus and Eschericia coli, and from the plant species Vitis vnifera, Rosa hybrida and Petunia hybrida.

FIG. 13 is a diagrammatic representation of the vector pSPB3855 containing an e35S: sense rose PH1: antisense rose PH1: mas expression cassette. Selected restriction endonuclease recognition sites are marked. The Gateway system (Invitrogen) was used to construct this plasmid.

### DETAILED DESCRIPTION

Nucleic acid sequences encoding polypeptides having pH modulating or altering activities have been identified, cloned and assessed. The nucleic acid sequence corresponds to the gene, PH1. This is a cation translocator. Reference to “PH1” includes the gene and its expression product (PH1 protein). It also encompasses homologs, orthologs, paralogs, polymorphic variants and derivatives of PH1 from any plant species. PH1 genetic sequences described herein permit the modulation of expression of this gene or altering its expression activities by, for example, de novo expression, over-expression, sense suppression, antisense inhibition, ribozyme, minizyme and DNAzyme activity, RNAi-induction or methylation-induction or other transcriptional or post-transcriptional silencing activities. RNAi-induction includes genetic molecules such as hairpin, short double stranded DNA or RNA, and partially double stranded DNAs or RNAs with one or two single stranded nucleotide overhangs. The ability to control cellular pH and in particular vacuolar pH in plants thereby enables the manipulation of petal color in response to pH change. A pH change can also lead to altered taste and flavor in tissues such as fruit including berries and other reproductive material. Moreover, plants and reproductive or vegetative parts thereof are contemplated herein including flowers, fruits, seeds, vegetables, leaves, stems and the like having altered levels of alkalinity or acidity. Other aspects include ornamental transgenic or genetically modified plants. The term “transgenic” also includes vegetative propagants and progeny plants and plants from subsequent genetic manipulation and/or crosses thereof from the primary transgenic plants.

The present invention extends to manipulating PH1 alone or in combination with one or more of altering levels of PH5, F3′5′H, F3′H, DFR, MT and a sodium-potassium antiporter or other ion transporter mechanism for the purposes of altering flower color and other infloresence and/or taste or flavor of fruit including berries and other reproductive material.

Reference to “MT” means an MT which acts on anthocyanin.

Hence, the present invention encompasses manipulating levels of PH1 alone or in combination with one or more of PH5, F3′5′H, F3′H, DFR, MT and an ion transporter for the purposes of altering flower color and other infloresence and/or taste or flavor of fruit including berries and other reproductive material.

Accordingly, the present invention provides an isolated nucleic acid molecule comprising a sequence of nucleotides encoding or complementary to a sequence encoding a pH modulating or altering gene or a polypeptide having the pH modulating or altering characteristics of PH1 wherein expression of the nucleic acid molecule alters or modulates pH inside the cell. In one aspect, the pH is altered in the vacuole.

More particularly, an isolated nucleic acid molecule corresponding to PH1 is provided comprising a sequence of nucleotides encoding or corresponding to PH1 wherein expression of PH1 alters or modulates pH inside the cell. PH1 expression leads to a lowering of pH to acidic conditions. A decrease in PH1 levels or acticity results in an increase in pH to more alkaline conditions.

As indicated above, in a particular embodiment, the nucleic acid modulates vacuolar pH. In particular, decreasing PH1 alone or in combination with PH5 results in alkaline conditions. In another embodiment, increasing PH1 alonge or in combination with PH5 results in more acidic conditions. By increasing or decreasing PH1 or PH5 is meant increasing or decreasing the level of protein or protein activity. Altered pH can lead to altered flower color or other characteristics such as taste and flavor in tissues such as fruit including berries and other reproductive material.

Another aspect contemplates an isolated nucleic acid molecule comprising a sequence of nucleotides encoding or corresponding to PH1 operably linked to a promoter.

Homologous PH1 nucleic acid molecules and proteins derived from rose, petunia, grape and carnation are particularly contemplated. A “PH1” includes all homologs, orthologs, paralogs, polymorphic variants and derivatives (naturally occurring or artificially induced). In a further embodiment, a PH1 is considered herein as capable of complementing a plant which lacks the function of the PH1 gene. Hence, contemplated herein is a PH1 nucleic acid molecule capable of restoring PH1 activity or function in a cell or organelle. In a particular embodiment, the PH1 can complement a ph1 mutant petunia plant.

Reference to “derived” in relation to the nucleic acid molecule from a plant means isolated directly from the plant, is obtainable from a plant, is obtained indirectly via a nucleic acid library in a virus, bacterium or other cell or was originally from the plant but is maintained by a different plant.

By the term “nucleic acid molecule” is meant a genetic sequence in a non-naturally occurring condition. Generally, this means isolated away from its natural state or synthesized or derived in a non-naturally-occurring environment. More specifically, it includes nucleic acid molecules formed or maintained in vitro, including genomic DNA fragments recombinant or synthetic molecules and nucleic acids in combination with heterologous nucleic acids. It also extends to the genomic DNA or cDNA or part thereof encoding pH modulating sequences or a part thereof in reverse orientation relative to its own or another promoter. It further extends to naturally occurring sequences following at least a partial purification relative to other nucleic acid sequences.

The term “genetic sequence” is used herein in its most general sense and encompasses any contiguous series of nucleotide bases specifying directly, or via a complementary series of bases, a sequence of amino acids in a pH modulating protein and in particular PH1. Such a sequence of amino acids may constitute a full-length PH1 enzyme such as is set forth in SEQ ID NO:2 (Rosa hybrida) or 4 (Petunia hybrida), 43 (Vitis vinifera cv Pinot Noir) or 45 (Vitis vinifera cv Nebbiolo) or an amino acid sequence having at least 50% similarity thereto, or an active truncated form thereof or may correspond to a particular region such as an N-terminal, C-terminal or internal portion of the PH1 enzyme. An enzyme with 50% similarity to SEQ ID NOs:2, 4, 43 and/or 46 is one which can complement a PH1 mutant plant lacking a functional PH1 or its homolog. In an embodiment, the PH1 DNA can complement a petunia ph1 mutant. A genetic sequence may also be referred to as a sequence of nucleotides or a nucleotide sequence and includes a recombinant fusion of two or more sequences.

In accordance with the above aspects of the present invention there is provided a nucleic acid molecule having the characteristics of PH1 comprising a nucleotide sequence or complementary nucleotide sequence substantially as set forth in SEQ ID NO:1 or 3 or 42 or 44 or 58 or 59 or having at least about 50% similarity to one or more of these sequences or capable of hybridizing to the sequence set forth in SEQ ID NO:1 or 3 or 42 or 44 or 58 or 59 under low stringency conditions. Hence, the present invention provides PH1 which is conveniently defined by and has the characteristics of modulating cellular and in particular vacuolar pH and which comprises an amino acid sequence having at least 50% similarity to one or more of SEQ ID NOs:2, 4, 43 and/or 45. Alternatively, the PH1 is characterized as being encoded by a nucleotide sequence having at least 50% identity to one or more of SEQ ID NOs:1, 3, 42, 44, 58 and/or 59 or a nucleotide sequence which hybridizes to the complement of SEQ ID NOs:1, 3, 42, 44, 58 and/or 59 under low stringency conditions. Hybridization conditions may also be defined in terms of medium or high stringency conditions. Still another alternative, the PH1 as defined above is capable of complementing a mutant incapable of producing a functional PH1 or its homolog. In an embodiment, the PH1 can complement a petunia ph1 mutant.

Alternative percentage similarities and identities (at the nucleotide or amino acid level) encompassed by the present invention include at least about 60% or at least about 65% or at least about 70% or at least about 75% or at least about 80% or at least about 85% or at least about 90% or above, such as about 95% or about 96% or about 97% or about 98% or about 99%, such as at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In a particular embodiment, there is provided an isolated nucleic acid molecule comprising a nucleotide sequence or complementary nucleotide sequence substantially as set forth in SEQ ID NO:1 or 3 or 42 or 44 or 58 or 59 or having at least about 50% similarity thereto or capable of hybridizing to a complementary sequence of SEQ ID NO:1 or 3 or 42 or 44 or 58 or 59 under low stringency conditions, wherein said nucleotide sequence encodes PH1 having pH modulating or altering activity. In an embodiment, a nucleic acid sequence set forth in SEQ ID NO:1 or 3 or 42 or 44 or 58 or 59 or having 50% similarity to one or more of these sequences or which can hybridize to one or more of these sequences under low stringency conditions is capable of complementing a PH1 mutant from the same species from which the nucleotide sequence is isolated or obtained. Hence, for example, rose PH1 is capable of restoring a mutant rose incapable of producing PH1. In another embodiment, PH1 or PH1 homolog is capable of functionally complementing a petunia ph1 mutant.

For the purposes of determining the level of stringency to define nucleic acid molecules capable of hybridizing to SEQ ID NO:1 or 3 or 42 or 44 or 58 or 59 reference herein to a low stringency includes and encompasses from at least about 0% to at least about 15% v/v formamide and from at least about 1M to at least about 2 M salt for hybridization, and at least about 1 M to at least about 2 M salt for washing conditions. Generally, low stringency is from about 25-30° C. to about 42° C. The temperature may be altered and higher temperatures used to replace the inclusion of formamide and/or to give alternative stringency conditions. Alternative stringency conditions may be applied where necessary, such as medium stringency, which includes and encompasses from at least about 16% v/v to at least about 30% v/v formamide and from at least about 0.5 M to at least about 0.9 M salt for hybridization, and at least about 0.5 M to at least about 0.9 M salt for washing conditions, or high stringency, which includes and encompasses from at least about 31% v/v to at least about 50% v/v formamide and from at least about 0.01 M to at least about 0.15 M salt for hybridization, and at least about 0.01 M to at least about 0.15 M salt for washing conditions. In general, washing is carried out Tₘ=69.3+0.41 (G+C) % (Marmur and Doty, J. Mol. Biol. 5: 109, 1962). However, the Tₘ of a duplex DNA decreases by 1° C. with every increase of 1% in the number of mismatch base pairs (Bonner and Laskey, Eur. J. Biochem. 46: 83, 1974). Formamide is optional in these hybridization conditions. Accordingly, particularly preferred levels of stringency are defined as follows: low stringency is 6×SSC buffer, 1.0% w/v SDS at 25-42° C.; a moderate stringency is 2×SSC buffer, 1.0% w/v SDS at a temperature in the range 20° C. to 65° C.; high stringency is 0.1×SSC buffer, 0.1% w/v SDS at a temperature of at least 65° C.

Another aspect of the present invention provides a nucleic acid molecule comprising a sequence of nucleotides encoding or complementary to a sequence encoding an amino acid sequence substantially as set forth in SEQ ID NO:2 or 4 or 43 or 45 or an amino acid sequence having at least about 50% similarity thereto after optimal alignment.

The term similarity as used herein includes exact identity between compared sequences at the nucleotide or amino acid level. Where there is non-identity at the nucleotide level, similarity includes differences between sequences which result in different amino acids that are nevertheless related to each other at the structural, functional, biochemical and/or conformational levels. Where there is non-identity at the amino acid level, similarity includes amino acids that are nevertheless related to each other at the structural, functional, biochemical and/or conformational levels. In a particular embodiment, nucleotide sequence comparisons are made at the level of identity and amino acid sequence comparisons are made at the level of similarity.

Terms used to describe sequence relationships between two or more polynucleotides or polypeptides include “reference sequence”, “comparison window”, “sequence similarity”, “sequence identity”, “percentage of sequence similarity”, “percentage of sequence identity”, “substantially similar” and “substantial identity”. A “reference sequence” is at least 12 but frequently 15 to 18 and often at least 25 or above, such as 30 monomer units, inclusive of nucleotides and amino acid residues, in length. Because two polynucleotides may each comprise (1) a sequence (i.e. only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of typically 12 contiguous residues that is compared to a reference sequence. The comparison window may comprise additions or deletions (i.e. gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e. resulting in the highest percentage homology over the comparison window) generated by any of the various methods selected. Reference also may be made to the BLAST family of programs as, for example, disclosed by Altschul et al, (Nucl. Acids Res. 25: 3389-3402, 1997). A detailed discussion of sequence analysis can be found in Unit 19.3 of Ausubel et al, Current Protocols in Molecular Biology John Wiley & Sons Inc, 1994-1998, Chapter 15, 1998.

The terms “sequence similarity” and “sequence identity” as used herein refers to the extent that sequences are identical or functionally or structurally similar on a nucleotide-by-nucleotide basis or an amino acid-by-amino acid basis over a window of comparison. Thus, a “percentage of sequence identity”, for example, is calculated by comparing two optimally aligned sequences over the window of comparison, determining the number of positions at which the identical nucleic acid base (e.g. A, T, C, G, I) or the identical amino acid residue (e.g. Ala, Pro, Ser, Thr, Gly, Val, Leu, Ile, Phe, Tyr, Trp, Lys, Arg, H is, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software. Similar comments apply in relation to sequence similarity.

The nucleic acid sequences contemplated herein also encompass oligonucleotides useful as genetic probes for amplification reactions or as antisense or sense molecules capable of regulating expression of the corresponding PH1 gene in a plant. Sense molecules include hairpin constructs, short double stranded DNAs and RNAs and partially double stranded DNAs and RNAs which one or more single stranded nucleotide over hangs. An antisense molecule as used herein may also encompass a genetic construct comprising the structural genomic or cDNA gene or part thereof in reverse orientation relative to its own or another promoter. It may also encompass a homologous genetic sequence. An antisense or sense molecule may also be directed to terminal or internal portions of the PH1 gene such that the expression of the gene is reduced or eliminated.

With respect to this aspect, there is provided an oligonucleotide of 5-50 nucleotides such as 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55 having substantial similarity to a part or region of a molecule with a nucleotide sequence set forth in SEQ ID NO:1 or 3 or 42 or 44 or 58 or 59 or a PH1 homolog having at least 50% identity to SEQ ID NO:1 or 3 or 5 or which hybridizes to a complementary strand of SEQ ID NO:1 or 3 or 42 or 44 or 58 or 59 under low stringency conditions. By substantial similarity or complementarity in this context is meant a hybridizable similarity under low, alternatively and preferably medium and alternatively and most preferably high stringency conditions specific for oligonucleotide hybridization (Sambrook et al, Molecular Cloning: A Laboratory Manual, 2^{nd} edition, Cold Spring Harbor Laboratories, Cold Spring Harbor, N.Y., USA, 1989). Such an oligonucleotide is useful, for example, in screening for pH modulating or altering genetic sequences from various sources or for monitoring an introduced genetic sequence in a transgenic plant. One particular oligonucleotide is directed to a conserved pH modulating or altering genetic sequence or a sequence within PH1.

In one aspect, the oligonucleotide corresponds to the 5′ or the 3′ end of PH1. For convenience, the 5′ end is considered herein to define a region substantially between the start codon of the structural gene to a center portion of the gene, and the 3′ end is considered herein to define a region substantially between the center portion of the gene and the terminating codon of the structural gene. It is clear, therefore, that oligonucleotides or probes may hybridize to the 5′ end or the 3′ end or to a region common to both the 5′ and the 3′ ends. The present invention extends to all such probes.

In one embodiment, the nucleic acid sequence encoding PH1 or various functional derivatives thereof is used to reduce the level of an endogenous PH1 (e.g. via co-suppression or antisense-mediated suppression) or other post-transcriptional gene silencing (PTGS) processes including RNAi or alternatively the nucleic acid sequence encoding this enzyme or various derivatives or parts thereof is used in the sense or antisense orientation to reduce the level of a pH modulating or altering protein. The use of sense strands, double or partially single stranded such as constructs with hairpin loops is particularly useful in inducing a PTGS response. In a further alternative, ribozymes, minizymes or DNAzymes could be used to inactivate target nucleic acid sequences.

Still a further embodiment encompasses post-transcriptional inhibition to reduce translation into PH1 polypeptide material. Still yet another embodiment involves specifically inducing or removing methylation.

Reducing PH1 levels or activity leads to an increase in pH leading to alkaline conditions.

Reference herein to the changing of a pH modulating or altering activity relates to an elevation or reduction in activity of up to 30% or more preferably of 30-50%, or even more preferably 50-75% or still more preferably 75% or greater above or below the normal endogenous or existing levels of activity. Such elevation or reduction may be referred to as modulation or alteration of PH1. Often, modulation is at the level of transcription or translation of PH1. Alternatively, changing pH modulation is measured in terms of degree of alkalinity or acidity and/or an ability to complement a PH1 mutant plant such as a ph1 petunia mutant.

The nucleic acids of the present invention encoding or controlling PH1 may be a ribonucleic acid or deoxyribonucleic acids, single or double stranded and linear or covalently closed circular molecules. Generally, the nucleic acid molecule is cDNA. The present invention also extends to other nucleic acid molecules which hybridize under low, particularly under medium and most particularly under high stringency conditions with the nucleic acid molecules of the present invention and in particular to the sequence of nucleotides set forth in SEQ ID NO:1 or 3 or 42 or 44 or 58 or 59 or a part or region thereof. In a particular embodiment, a nucleic acid molecule is provided having a nucleotide sequence set forth in SEQ ID NO:1 or 3 or 42 or 44 or 58 or 59 or to a molecule having at least 50%, more particularly at least 55%, still more particularly at least 65%-70%, and yet even more preferably greater than 85% similarity at the nucleotide level to at least one or more regions of the sequence set forth in SEQ ID NO:1 or 3 or 42 or 44 or 58 or 59 and wherein the nucleic acid encodes or is complementary to a sequence which encodes PH1. It should be noted, however, that nucleotide or amino acid sequences may have similarities below the above given percentages and yet still encode a PH1 homolog or derivative and such molecules are still considered to be within the scope of the present invention where they have regions of sequence conservation.

The term gene is used in its broadest sense and includes cDNA corresponding to the exons of a gene. Accordingly, reference herein to a gene is to be taken to include:—

(i) a classical genomic gene consisting of transcriptional and/or translational regulatory sequences and/or a coding region and/or non-translated sequences (i.e. introns, 5′- and 3′-untranslated sequences); or
(ii) mRNA or cDNA corresponding to the coding regions (i.e. exons) and 5′- and 3′-untranslated sequences of the gene.

The term gene is also used to describe synthetic or fusion molecules encoding all or part of an expression product. In particular embodiments, the term nucleic acid molecule and gene may be interchangeably used.

The nucleic acid or its complementary form may encode the full-length PH1 enzyme or a part or derivative thereof. By “derivative” is meant any single or multiple amino acid substitutions, deletions, and/or additions relative to the naturally occurring enzyme and which retains a pH modulating or altering activity and/or an ability to complement a PH1 mutant plant or plant tissue such as a petunia ph1 mutant plant. In this regard, the nucleic acid includes the naturally occurring nucleotide sequence encoding a pH modulating or altering activity or may contain single or multiple nucleotide substitutions, deletions and/or additions to the naturally occurring sequence. The nucleic acid of the present invention or its complementary form may also encode a “part” of the pH modulating or altering protein, whether active or inactive, and such a nucleic acid molecule may be useful as an oligonucleotide probe, primer for polymerase chain reactions or in various mutagenic techniques, or for the generation of antisense molecules.

Reference herein to a “part” of a nucleic acid molecule, nucleotide sequence or amino acid sequence, preferably relates to a molecule which contains at least about 10 contiguous nucleotides or five contiguous amino acids, as appropriate.

Amino acid insertional derivatives of the pH modulating or altering protein of the present invention include amino and/or carboxyl terminal fusions as well as intra-sequence insertions of single or multiple amino acids. Insertional amino acid sequence variants are those in which one or more amino acid residues are introduced into a predetermined site in the protein although random insertion is also possible with suitable screening of the resulting product. Deletional variants are characterized by the removal of one or more amino acids from the sequence. Substitutional amino acid variants are those in which at least one residue in the sequence has been removed and a different residue inserted in its place. Typical substitutions are those made in accordance with Table 2.

TABLE 2
Suitable residues for amino acid substitutions

| Original residue | Exemplary substitutions |
|---|---|
| Ala | Ser |
| Arg | Lys |
| Asn | Gln; His |
| Asp | Glu |
| Cys | Ser |
| Gln | Asn; Glu |
| Glu | Asp |
| Gly | Pro |
| His | Asn; Gln |
| Ile | Leu; Val |
| Leu | Ile; Val |
| Lys | Arg; Gln; Glu |
| Met | Leu; Ile; Val |
| Phe | Met; Leu; Tyr |
| Ser | Thr |
| Thr | Ser |
| Trp | Tyr |
| Tyr | Trp; Phe |
| Val | Ile; Leu; Met |

Where PH1 protein is derivatized by amino acid substitution, the amino acids are generally replaced by other amino acids having like properties, such as hydrophobicity, hydrophilicity, electronegativity, bulky side chains and the like. Amino acid substitutions are typically of single residues. Amino acid insertions will usually be in the order of about 1-10 amino acid residues and deletions will range from about 1-20 residues. Generally, deletions or insertions are made in adjacent pairs, i.e. a deletion of two residues or insertion of two residues.

The amino acid variants referred to above may readily be made using peptide synthetic techniques well known in the art, such as solid phase peptide synthesis (Merrifield, J. Am. Chem. Soc. 85:2149, 1964) and the like, or by recombinant DNA manipulations. Techniques for making substitution mutations at predetermined sites in DNA having known or partially known sequence are well known and include, for example, M13 mutagenesis. The manipulation of DNA sequence to produce variant proteins which manifest as substitutional, insertional or deletional variants are conveniently described, for example, in Sambrook et al, 1989 supra.

Other examples of recombinant or synthetic mutants and derivatives of PH1 described herein include single or multiple substitutions, deletions and/or additions of any molecule associated with the enzyme such as carbohydrates, lipids and/or proteins or polypeptides.

The terms “homologs”, “orthologs”, “paralogs”, “polymorphic variants” and “derivatives” also extend to any functional equivalent of PH1 and also to any amino acid derivative described above. For convenience, reference to PH1 herein includes reference to any functional mutant, derivative, part, fragment or homolog thereof.

Nucleic acid sequences derived from rose, petunia, grape and carnation are particularly contemplated herein since this represents a convenient source of material to date. However, one skilled in the art will immediately appreciate that similar sequences can be isolated from any number of sources such as other plants or certain microorganisms. All such nucleic acid sequences encoding directly or indirectly a PH1 are encompassed herein regardless of their source. Examples of other suitable sources of genes encoding PH1 include, but are not limited to Liparieae, Plumbago spp, Gerbera spp, Chrysanthemum spp, Dendranthema spp, lily, Gypsophila spp, Torenia spp, orchid, Cymbidium spp, Dendrobium spp, Phalaenopsis spp, cyclamen, Begonia spp, Iris spp, Alstroemeria spp, Anthurium spp, Catharanthus spp, Dracaena spp, Erica spp, Ficus spp, Freesia spp, Fuchsia spp, Geranium spp, Gladiolus spp, Helianthus spp, Hyacinth spp, Hypericum spp, Impatiens spp, Iris spp, Chamelaucium spp, Kalanchoe spp, Lisianthus spp, Lobelia spp, Narcissus spp, Nierembergia spp, Ornithoglaum spp, Osteospermum spp, Paeonia spp, Pelargonium spp, Primrose spp, Ruscus spp, Saintpaulia spp, Solidago spp, Spathiphyllum spp, Tulip spp, Verbena spp, Zantedeschia spp etcanenome, hyacinth, Liatrus spp, Viola spp, Nierembergia spp and Nicotiana spp, etc.

Hence, in an aspect of the present invention a PH1 homolog is provided which complements a PH1 mutant in a plant selected from Rosa spp, Vitis spp, Dianthus spp, Petunia spp, Liparieae, Plumbago spp, Gerbera spp, Chrysanthemum spp, Dendranthema spp, lily, Gypsophila spp, Torenia spp, orchid, Cymbidium spp, Dendrobium spp, Phalaenopsis spp, cyclamen, Begonia spp, Iris spp, Alstroemeria spp, Anthurium spp, Catharanthus spp, Dracaena spp, Erica spp, Ficus spp, Freesia spp, Fuchsia spp, Geranium spp, Gladiolus spp, Helianthus spp, Hyacinth spp, Hypericum spp, Impatiens spp, Iris spp, Chamelaucium spp, Kalanchoe spp, Lisianthus spp, Lobelia spp, Narcissus spp, Nierembergia spp, Ornithoglaum spp, Osteospermum spp, Paeonia spp, Pelargonium spp, Primrose spp, Ruscus spp, Saintpaulia spp, Solidago spp, Spathiphyllum spp, Tulip spp, Verbena spp, Zantedeschia spp etcanenome, hyacinth, Liatrus spp, Viola spp, Nierembergia spp and Nicotiana spp. More particularly, the PH1 or homolog complements a petunia ph1 mutant.

A nucleic acid sequence is described herein encoding PH1 may be introduced into and expressed in a transgenic plant in either orientation thereby providing a means to modulate or alter the vacuolar pH by either reducing or eliminating endogenous or existing pH modulating or altering protein activity thereby allowing the vacuolar pH to increase. A particular effect is a visible effect of a shift to blue in the color of the anthocyanins and/or in the resultant flower color. There may also be a change in taste or flavor. In particular, the taste or flavor change in fruit including berries and other reproductive material. Expression of the nucleic acid sequence in the plant may be constitutive, inducible or developmental and may also be tissue-specific. The word “expression” is used in its broadest sense to include production of RNA or of both RNA and protein. It also extends to partial expression of a nucleic acid molecule.

According to this aspect, there is provided a method for producing a transgenic flowering plant having altered levels of PH1, the method comprising stably transforming a cell of a suitable plant with a nucleic acid sequence which comprises a sequence of nucleotides encoding or corresponding to PH1 under conditions permitting the eventual expression of the nucleic acid sequence, regenerating a transgenic plant from the cell and growing the transgenic plant for a time and under conditions sufficient to permit the expression of the nucleic acid sequence. The transgenic plant may thereby produce non-indigenous PH1 at elevated levels relative to the amount expressed in a comparable non-transgenic plant. Alternatively, through mechanisms such as sense suppression, indigenous levels of PH1 may be reduced. It is proposed herein that reduced PH1 levels leads to more alkaline conditions and an elevated PH1 leads to more acidic conditions.

Another aspect contemplates a method for producing a transgenic plant with reduced indigenous or existing PH1 levels, the method comprising stably transforming a cell of a suitable plant with a nucleic acid molecule which comprises a sequence of nucleotides encoding or corresponding to PH1, regenerating a transgenic plant from the cell and where necessary growing the transgenic plant under conditions sufficient to permit the expression of the nucleic acid. Such a plant may be a transgenic plant or the progeny of a transgenic plant. Progeny of transgenic plants contemplated herein are nevertheless still genetically modified and exhibit increased alkalinity by levels or organelles.

Yet another aspect provides a method for producing a genetically modified plant with reduced indigenous or existing PH1 activity, the method comprising altering the PH1 gene through modification of the indigenous sequences via homologous recombination from an appropriately altered PH1 introduced into the plant cell, and regenerating the genetically modified plant from the cell and optionally generating genetically modified progeny therefrom.

Still another aspect contemplates a method for producing a genetically modified plant with reduced indigenous PH1 protein activity, the method comprising altering PH1 levels by reducing expression of a gene encoding the indigenous PH1 protein by introduction of a nucleic acid molecule into the plant cell and regenerating the genetically modified plant from the cell and optionally generating genetically modified progeny therefrom.

Yet another aspect provides a method for producing a transgenic plant capable of generating a pH altering protein, the method comprising stably transforming a cell of a suitable plant with the PH1 nucleic acid molecule obtainable from rose, petunia or carnation comprising a sequence of nucleotides encoding, or complementary to, a sequence encoding PH1 and regenerating a transgenic plant from the cell and optionally generating genetically modified progeny therefrom.

Hence, relation to these aspects, the method may further involve generating progeny which exhibit the genetic trait associated with PH1.

As used herein an “indigenous” enzyme is one, which is native to or naturally expressed in a particular cell. A “non-indigenous” enzyme is an enzyme not native to the cell but expressed through the introduction of genetic material into a plant cell, for example, through a transgene. An “endogenous” enzyme is an enzyme produced by a cell but which may or may not be indigenous to that cell.

The term “inflorescence” as used herein refers to the flowering part of a plant or any flowering system of more than one flower which is usually separated from the vegetative parts by an extended internode, and normally comprises individual flowers, bracts and peduncles, and pedicels. As indicated above, reference to a “transgenic plant” may also be read as a “genetically modified plant”. A “genetically modified plant” includes modified progeny from the originally produced transgenic plant.

Alternatively, the method may comprise stably transforming a cell of a suitable plant with PH1 nucleic acid sequence or its complementary sequence, regenerating a transgenic plant from the cell and growing the transgenic plant for a time and under conditions sufficient to alter the level of activity of the indigenous or existing PH1. In one embodiment, the altered level would be less than the indigenous or existing level of PH1 in a comparable non-transgenic or mutant plant. In another embodiment, the altered level is more than the indigenous or existing level of PH1 in a comparable non-transgenic or mutant plant decreasing or increasing Ph1 levels leads to a flowering plant exhibiting altered floral or inflorescence properties or altered other properties such as taste or flavor of fruit including berries or other reproductive material.

In a related embodiment, a method is provided for producing a flowering plant exhibiting altered floral or inflorescence properties, the method comprising alteration of the level of PH1 gene expression to either decrease the level of PH1 or increase the level of Ph1 wherein a decrease in Ph1 leads to more alkaline conditions and an increase in PH1 leads to more acidic conditions and regenerating a transgenic plant and optionally generating genetically modified progeny therefrom.

In a particular aspect, the altered floral or inflorescence includes the production of different shades of blue or purple or red flowers or other colors, depending on the genotype and physiological conditions of the recipient plant. In another aspect, there is an alteration in taste or flavor in tissues such as fruit including berries or other reproductive material.

Accordingly, a method is contemplated for producing a transgenic plant capable of expressing a recombinant PH1 gene or part thereof or which carries a nucleic acid sequence which is substantially complementary to all or a part of a mRNA molecule encoding PH1, the method comprising stably transforming a cell of a suitable plant with the isolated nucleic acid molecule comprising a sequence of nucleotides encoding, or complementary to a sequence encoding PH1, where necessary under conditions permitting the eventual expression of the isolated nucleic acid molecule, and regenerating a transgenic plant from the cell and optionally generating genetically modified porgeny from the transgenic plant. The plant may also be genetically engineered to alter levels of or introduce de novo levels of an F3′5′H, F3′H, DFR and/or MT or other enzymes of the anthocyanin pathway.

In addition, the activity of PH5 or other pH modulating gene or an ion transporter may be modulated.

The cellular and in particular vascular pH may be manipulated by PH1 alone or in combination with PH5. PH5 is described in International Patent Applications PCT/AU2006/000451 and PCT/AU2007/000739. The anthocyanin pathway genes optionally contemplated to be used in conjunction with PH1 (an optionally PH5) have been previously described, for example, in patents and patent application for the families relating to PCT/AU92/00334; PCTAU96/00296; PCT/AU93/00127; PCT/AU97/00124; PCT/AU93/00387; PCT/AU93/00400; PCT/AU01/00358; PCT/AU03/00079; PCT/AU03/01111 and JP 2003-293121, the contents of all of which are incorporated by reference. These genes include inter alia F3′,5′H, F3′H, DFR, PH5 and MT.

It is proposed that PH1 alone or in combination with PH5 and/or transporters which use proton gradients to transport large molecules (e.g. MATE transporters which exchange protons for proanthocyanins) or ions, such as NHX (which exchanges protons for Na⁺ or K⁺) promotes a higher level of sequestration of specific molecules in the vacuolar lumen. This is for the purpose of altering flower color and other infloresence and/or taste or flavor of fruit including berries and other reproductive material It is further proposed herein that vacuolar pH affects root absorption and stomata opening which influences wilting of flowers and plants.

In addition, anthocyanin genes may be manipulated along with PH1 and optionally PH5.

One skilled in the art will immediately recognize the variations applicable to the methods described herein, such as increasing or decreasing the expression of the enzyme naturally present in a target plant leading to differing shades of colors such as different shades of blue, purple or red, or changing taste or flavor in tissues such as fruit including berries or other reproductive material.

The instant disclosure, therefore, extends to all transgenic plants or parts or cells therefrom of transgenic plants or genetically modified progeny of the transgenic plants containing all or part of the nucleic acid sequences of the present invention, or antisense forms thereof and/or any homologs or related forms thereof and, in particular, those transgenic plants which exhibit altered floral or inflorescence properties. The transgenic plants may contain an introduced nucleic acid molecule comprising a nucleotide sequence encoding or complementary to a sequence encoding PH1. Generally, the nucleic acid would be stably introduced into the plant genome, although the present invention also extends to the introduction of PH1 within an autonomously-replicating nucleic acid sequence such as a DNA or RNA virus capable of replicating within the plant cell. This aspect also extends to seeds from such transgenic plants. Such seeds, especially if colored, are useful as proprietary tags for plants. Any and all methods for introducing genetic material into plant cells including but not limited to Agrobacterium-mediated transformation, biolistic particle bombardment etc. are encompassed herein.

Another aspect contemplates the use of the extracts from transgenic plants or plant parts or cells therefrom of transgenic plants or progeny of the transgenic plants containing all or part of the nucleic acid sequences described herein such as when used as a flavoring or food additive or health product or beverage or juice or coloring.

Plant parts contemplated herein include, but are not limited to flowers, fruits, vegetables, nuts, roots, stems, leaves or seeds. Such tissues are proposed to have altered pH levels or have a taste or flavor altered because of a change in pH levels. In particular, taste or flavor changes may occur in fruit including berries or other reproductive material.

The extracts may be derived from the plants or plant part or cells therefrom in a number of different ways including but not limited to chemical extraction or heat extraction or filtration or squeezing or pulverization.

The plant, plant part or cells therefrom or extract can be utilized in any number of different ways such as for the production of a flavoring (e.g. a food essence), a food additive (e.g. a stabilizer, a colorant) a health product (e.g. an antioxidant, a tablet) a beverage (e.g. wine, spirit, tea) or a juice (e.g. fruit juice) or coloring (e.g. food coloring, fabric coloring, dye, paint, tint).

A further aspect is directed to recombinant forms of PH1. The recombinant forms of the enzyme provide a source of material for research, for example, more active enzymes and may be useful in developing in vitro systems for production of colored compounds.

Still a further aspect contemplates the use of the genetic sequences described herein such as from rose in the manufacture of a genetic construct capable of expressing PH1 or down-regulating an indigenous PH1 in a plant.

The term genetic construct has been used interchangeably throughout the specification and claims with the terms “fusion molecule”, “recombinant molecule”, “recombinant nucleotide sequence”. A genetic construct may include a single nucleic acid molecule comprising a nucleotide sequence encoding a single protein or may contain multiple open reading frames encoding two or more proteins. It may also contain a promoter operably linked to one or more of the open reading frames.

Another aspect is directed to a prokaryotic or eukaryotic organism carrying a genetic sequence encoding PH1 extrachromasomally in plasmid form.

A “recombinant polypeptide” means a polypeptide encoded by a nucleotide sequence introduced into a cell directly or indirectly by human intervention or into a parent or other relative or precursor of the cell. A recombinant polypeptide may also be made using cell-free, in vitro transcription systems. The term “recombinant polypeptide” includes an isolated polypeptide or when present in a cell or cell preparation. It may also be in a plant or parts of a plant regenerated from a cell which produces said polypeptide.

A “polypeptide” includes a peptide or protein and is encompassed by the term “enzyme”.

The recombinant polypeptide may also be a fusion molecule comprising two or more heterologous amino acid sequences.

Still yet another aspect contemplates PH1 linked to a nucleic acid sequence involved in modulating or altering the anthocyanin pathway.

Another aspect is direct to the use of a nucleic acid molecule encoding PH1 in the manufacture of a plant with an altered pH compared to the pH in a non-manufactured plant of the same species. In a particular embodiment, the vacuolar pH is altered.

The present invention provides, therefore, a PH1 or PH1 homolog for a plant which:
(i) comprises a nucleotide sequence which has at least 50% identity to SEQ ID NOs:1, 3, 42, 44, 58 or 59 after optimal alignment;
(ii) comprises a nucleotide sequence which is capable of hybridizing to SEQ ID NOs:1, 3, 42, 44, 58 or 59 or its complement;
(iii) encodes an amino acid sequence which has at least 50% similarity to SEQ ID NOs:2, 4, 43 or 45 after optimal alignment;
(iv) when expressed in a plant cell or organelle, leads to acidic conditions or when its expression is reduced in a plant cell or organelle, leads to alkaline conditions.

In an embodiment, the PH1 or its homolog is capable of complementing a PH1 mutant in the same species from which it is derived. In a particular embodiment, the PH1 can complement a ph1 mutant in petunia.

The present invention further contemplates the use of a PH1 or its homolog alone or in combination with PH5 and/or enzymes of the anthocyanin pathway as defined above in the manufacture of a transgenic plant or genetically modified progeny thereof exhibiting altered inflorescence or other characteristics such as taste or flavor.

The present invention is further described by the following non-limiting Examples.

In relation to these Examples, the following methods and agents are employed.

In general, the methods followed were as described in Sambrook et al, 1989 supra or Sambrook and Russell, Molecular Cloning: A Laboratory Manual 3^{rd} edition, Cold Spring Harbor Laboratories, Cold Spring Harbor, N.Y., USA, 2001 or Plant Molecular Biology Manual (2^{nd} edition), Gelvin and Schilperoot (eds), Kluwer Academic Publisher, The Netherlands, 1994 or Plant Molecular Biology Labfax, Croy (ed), Bios scientific Publishers, Oxford, UK, 1993.

### Petunia Plant Material

The Petunia hybrida lines used in the cDNA-AFLP screening were R27 (wild-type (wt)), W225 (an1, frame-shift mutation in R27 background), R144 (phi-V2068 transposon insertion in PH3 in R27 background), R147 (ph4-X2058 transposon insertion in PH4 in R27 background) and R153 (ph5 transposon insertion in PH5 crossed into a R27 background). All lines have genetically identical background and to diminish differences in environmental conditions which could lead to differences in transcript levels, the plants were grown in a greenhouse adjacent to each other.

The Petunia hybrida line M1×V30 used in transformation experiments was an F1 hybrid of M1 (AN1, AN2, AN4, PH4, PPM1, PPM2) crossed with line V30 (AN1, AN2, AN4, PH4, PPM1, PPM2). Flowers of M1×V30 are red-violet and generally accumulate anthocyanins based upon malvidin and low levels of the flavonol quercetin.

Furthermore, Petunia hybrida lines V63 X R149 (F1 hybrid of two different ph4-lines), V30 X V23 (F1 hybrid with wild-type phenotype) and R170 (F1 hybrid that contains a tagged ph1 allelle from L2164×R67) were used in various transformation experiments.

### Stages of Flower Development

Petunia hybrida cv. M1×V30 flowers were harvested at developmental stages defined as follows:

Stage 1: Unpigmented flower bud (less than 10 mm in length)
Stage 2: Unpigmented flower bud (10 to 20 mm in length)
Stage 3: Lightly pigmented closed flower bud (20 to 27 mm in length)
Stage 4: Pigmented closed flower bud (27 to 35 mm in length)
Stage 5: Fully pigmented closed flower bud (35 to 45 mm in length)
Stage 6: Fully pigmented bud with emerging corolla (45 to 55 mm in length)
Stage 7: Fully opened flower (55 to 60 mm in length)

Petunia cultivers V67, V23, V42 and V48 have mutated PH1 alleles. Other petunia cultivars (such as R27 and W115) were grouped into similar developmental stages.

Flowers of Rosa hybrida cv. Rote rose were obtained from a nursery in Kyoto, Japan.

Stages of Rosa hybrida flower development are defined as follows:
Stage 1: Unpigmented, tightly closed bud.
Stage 2: Pigmented, tightly closed bud.
Stage 3: Pigmented, closed bud; sepals just beginning to open.
Stage 4: Flower bud beginning to open; petals heavily pigmented; sepals have separated.
Stage 5: Sepals completely unfolded; some curling. Petals are heavily pigmented and unfolding.
   Petunia hybrida Transformations

As described in Holton et al, Nature 366:276-279, 1993 or Brugliera et al, Plant J. 5:81-92, 1994 or de Vetten Net al, Genes and Development 11:1422-1434, 1997 or by any other method well known in the art. One particular method is described below.

Leaf explants were taken either from in vitro cultivated plants or from plants growing in the greenhouse. For in vitro explant stocks, plants were maintained on 0.5×MS medium (Murashige and Skoog, Physiologia Plantarum 15:473-497, 1962) without plant growth regulators.

To transform lines (e.g. W115, V26, VR), leaves not fully expanded were taken from young plants from the greenhouse. Surface sterilization was achieved by immersing leaves in 70% v/v ethanol. This step was optional as it sometimes gave rise to necrosis, especially when very young leaves were used. In the case of necrosis occurring, the ethanol immersion step was omitted. Leaves were then incubated for 10 minutes in 0.5% v/v sodium hypochlorite followed by five rinses in sterile water within a period of 10 minutes.

Following sterilization, leaves were cut into explants of maximum 0.5×0.5 cm, ensuring all sides were wounded. Leaves were manipulated in a sterile petridish using a sharp scalpel.

Petunia growth medium referred to for petunia transformation contains the following components per 500 mL:
2.2 g MS-macro and micro elements (Murashige and Skoog, Physiologia Plantarum 15:473-497, 1962) with Gamborg B5 vitamins (Gamborg et al., Experimental Cell Research, 95:355-358, 1970) (Duchefa Catalog No. M 0231)
0.8% Micro Agar (Duchefa Catalog No. M 1002) or 0.4% Gelrite (Duchefa Catalog No. G1101)
2% sucrose*
1% glucose*
2.2 μM folic acid (Duchefa Catalog No. F 0608)
8.8 μM 6-benzyl amino purine (BAP; Duchefa Catalog No. B 0904)
0.5 μM naphthylacetic acid (NAA; Duchefa Catalog No. N 0903)
4.5 μM zeatin (1 mg/ml); optional for petunia (Duchefa Catalog No. Z 0917)

Petunia selection medium contains the above components with the addition of:
250 mg/l carbenicillin (for bacterial selection)
250 mg/l kanamycin, 20 mg/l hygromycin or 5 mg/l basta, dependant on transformation vector used (for plant selection)

The pH of the petunia growth medium was adjusted to 5.7-5.9, and the media autoclaved at 110° C. for 10 minutes. *To prevent aggregation of Gelrite before autoclaving, sucrose and glucose were added prior to the addition of water.

Plant growth regulators were present in growth medium during co-cultivation and selection, but were omitted from rooting medium.

Explants were placed in a sterile petridish containing 20-25 ml of a 1:10 diluted (in water) of overnight grown Agrobacterium tumefaciens culture (LBA 4404/EHA 105/AGL 0) containing 20 μM acetosyringone and incubated for 10-15 min. Explants were transferred to co-cultivation medium (petunia growth medium containing 20 μM acetosyringone; 20-30 explants per petridish) and incubated for 2-3 days at 25° C. under 16 h/8 h day/night photoperiod.

Following co-cultivation, explants were transferred to petunia selection medium (8-10 explants per petridish). Care was taken to ensure that the edges of the explants were in contact with the medium to ensure escapes did not occur. Explants were incubated at 25° C. under 16 h/8 h day/night photoperiod.

Plates were checked for fungi every one to two days in the first week of incubations. Infected explants were discarded.

Explants were transferred to fresh selection medium every three weeks. If shoots were not observed following 3 to 6 weeks incubation on selection medium, explants were transferred to either, selection medium without BAP and half the original concentration of NAA or, selection medium without BAP or NAA but containing 4.5 μM zeatin.

Shoots were excised and rooted on petunia selection medium without plant growth regulators. Roots appeared after 1 to 2 weeks.

Following root proliferation, the gelrite/agar was carefully removed from the roots using warm water. Plants were planted in jiffy compressed peat pellets or pots containing soil and grown in a high humidity environment in the greenhouse for 2 to 3 weeks to acclimatize and allow formation of mature functional roots.

Petunia hybrida Transient Transformations—Infiltration

One particular method is described below for the transient transformation of Petunia hybrida with GFP:PH1 fusion contructs using Agrobacterium infiltration.

Prior to commencing Agrobacterium infiltration, the target plant was sprayed with water to encourage opening of stomata.

Overnight grown Agrobacterium tumefaciens culture (LBA 4404/EHA 105/AGL 0) containing 20 μM acetosyringone was spun at 2500×g for 15 minutes. The resulting pellet was washed with infiltration solution and spun again at 2500×g for 10 minutes. The pellet was then resuspended in infiltration solution to an OD₆₀₀ₙₘ of 0.3.

Using a syringe (without needle), the Agrobacteriumn tumefaciens infiltration solution was applied to the abaxial side of the leaf using a small amount of pressure. This was repeated to different spots on the same leaf.

Following infiltration the plant was placed under light, or alternatively the infiltrated leaf was removed and its petiole inserted in a solidified MS contained in a Petri dish and the Petri dish placed under light. The following day transiently transformed cells could be visualized under UV light and magnification.

Petunia hybrida Transient Transformations—Vacuum Infiltration

One particular method is described below for the transient transformation of Petunia hybrida with GFP:PH fusion contructs using Agrobacterium vacuum infiltration.

Using the Agrobacteriumn tumefaciens infiltration solution described above, an entire leaf with associated petiole was submerged in 50-75 mL of solution and a vacuum applied. Once air bubbles were seen to be coming from the tissue, 5 minutes were counted then the vacuum released.

Infiltrated leaves were place on solidified MS contained in a Petri dish, with the petiole inserted in the agar, and the Petri dish placed under light. The following day transiently transformed cells could be visualized under UV light and magnification.

Petunia infiltration solution referred to for transient petunia transformation contains the following components:
50 mM MES pH 5.7
0.5% Glucose
2 mM Na₃PO₄
100 μM acetosyringone
   Preparation of Petunia R27 Petal cDNA Library

A petunia petal cDNA library was prepared from R27 petals using standard methods as described in Holton et al, 1993 supra or Brugliera et al, 1994 supra or de Vetten N et al, 1997 supra.

### Transient Assays

Transient expression assays were performed by particle bombardment of petunia petals as described previously (de Vetten et al, 1997 supra; Quattrocchio et al, Plant J. 13:475-488, 1998.

### pH Assay

The pH of petal extracts was measured by grinding the petal limbs of two corollas in 6 mL distilled water. The pH was measured within 1 min of sample preparation to avoid atmospheric CO₂ altering the pH of the extract,

### HPLC and TLC Analysis

HPLC analysis was as described in de Vetten et al, Plant Cell 11(8):1433-1444, 1999. TLC analysis was as described in van Houwelingen et al, Plant J. 13(1):39-50, 1998.

### Analysis of Nucleotide and Predicted Amino Acid Sequences

Unless otherwise stated, nucleotide and predicted amino acid sequences were analyzed with the program Geneworks (Intelligenetics, Mountain View, Calif.) or MacVector (Registered Trademark) application (version 6.5.3) (Oxford Molecular Ltd., Oxford, England). Multiple sequence alignments were produced with a web-based version of the program ClustalW (http://dot.imgen.bcm.tmc.edu:9331/multi-align/multi-align.html) using default parameters (Matrix=blossom; GAPOPEN=0, GAPEXT=0, GAPDIST=8, MAXDIV=40). Phylogenetic trees were built with PHYLIP (bootstrap count=1000) via the same website, and visualized with Treeviewer version 1.6.6 (http://taxonomy.zoology.gla.ac.uk/rod/rod.html).

Homology searches against Genbank, SWISS-PROT and EMBL databases were performed using the FASTA and TFASTA programs (Pearson and Lipman, Proc. Natl. Acad. Sci. USA 85(8): 2444-2448, 1988) or BLAST programs (Altschul et al., J. Mol. Biol. 215(3): 403-410, 1990). Percentage sequence identities and similarities were obtained using LALIGN program (Huang and Miller, Adv. Appl. Math. 12: 373-381, 1991) or ClustalW program (Thompson et al., Nucleic Acids Research 22: 4673-4680, 1994) within the MacVector (Registered Trademark) application (Oxford Molecular Ltd., England) using default parameters.

### RNA Isolation and RT-PCR

RNA isolation and RT-PCR analysis were carried out as described by de Vetten et al, 1997 supra. Rapid amplification of cDNA (3′) ends (RACE) was done as described by Frohman et al, PNAS 85:8998-9002, 1988.

### Constructs

Genetic constructs contain genomic clones from petunia, rose and grape. This is due to the fact that the cDNA cannot be cloned in bacteria as a result of toxicity. The rose PM was identified as described by using primers designed on the basis of sequence homologs with unknown function. The full size cDNA was obtained by RACE. By designing primers based on the sequence of the cDNA, a genomic fragment was amplified ranging from the ATG to the STOP. For the grape PH1, possible homologs were identified with grape genome and EST collection (Pinot Noir) by Blasting the petunia sequence. Primers were designed based on this sequence and a cDNA fragment amplified from berries of the Nebbiolo variety.

### Example 1

### Cloning of Petunia PH1

In the collection of petunia genotypes, four lines (R67, V23, V42 and V48) were known to harbor mutated alleles of the PH1 locus. Petunia plants mutant for ph1 produce flowers with bluish phenotype that can largely vary in intensity depending on the type of anthocyanin molecules accumulated in the petals. The pH value of the petal extracts from ph1 mutant petunia plants showed an increase of nearly one pH unit when compared to isogenic wild-type. The seed coat of ph1 mutants is normally colored and this is contrary to what has been observed in several other ph mutants, such as ph5, ph3, and ph4.

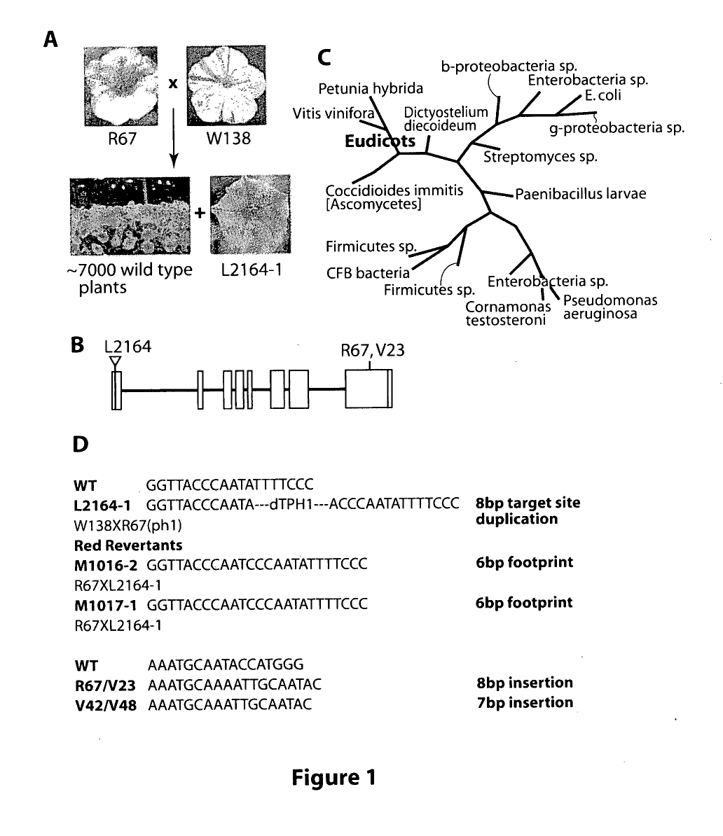

In order to tag the PH1 locus, a large number of crosses between the lines R67 and W138 (which carries a large number of active copies of the petunia transposon dTPH1) were produced. The screening of ˜7000 F1 progeny (all red) yielded one plant (L2164-1) with a ph mutant phenotype (purplish, FIG. 1).

Back cross of this plant to the line R67 (ph1^{R67}) resulted in plants displaying purple flowers and plants displaying purple flowers with red reversion spots. Two plants showed red (wild-type) flowers and possibly represented germinal revertants (PH1^{RevM1016} and PH1^{RevM}1017) of the tagged allele.

A transcript profile analysis of wild-type (WT) versus an1, ph3 and ph4 mutant flowers was performed. This yielded ˜15 cDNA fragments from genes whose expression was strongly reduced in all the mutants. For most of these genes, full size cDNA sequences were obtained and confirmed that their expression is under the control of AN1, PH3 and PH4.

Using primers designed from the sequence of these cDNAs, the possible presence of a transposon insertion was searched in the corresponding genomic fragment in the new, unstable ph1 mutant (plant L2164-1). The sequence corresponding to the differential cDNA named CAC7.5 (cDNA AFLP Clone 7.5 [Verweij, In Developmental Genetics (Amsterdam. Vrije Universiteit), 2007]) was amplified. Two PCR products were amplified from plant L2164-1, as well as from half of its back-cross progeny (with ph1 mutant lines). One of the two products was ˜300 by larger than that of wild-type related plants and of the germinal revertants isolated in the same backcross, consistent with the insertion of a copy of dTPH1 at this site. The other PCR product originated from a stable mutant ph1^{R67} allele (L2164-1 is an F1 of W138 and R67) and was the same size as the wild-type fragment. Sequence analysis showed the presence of a dTPH1 copy in the coding sequence of CAC7. 5 (13 by after the ATG of the predicted protein sequence) and of a 6 by footprint at the same position in the two revertant plants isolated from the backcross (FIG. 1D).

The ph1 alleles present in a collection of mutant petunia lines (ph1^{R}67, ph1^{V23}, ph1^{V42} and ph1^{V48}) were also characterized. These alleles all contained a different small insertion at the very same site (located at the end of the coding sequence, close to the STOP codon). ph1^{V23} possessed an 8 by insertion, while ph1^{V42} and ph1^{V48}, carrying the same allele (the two lines have probably a common origin), contained a 7 by insertion at this site. These alleles might originate from the excision of a transposon that inserted at this position and later moved away leaving behind a footprint (FIG. 1D).

The PH1 transcript is petal specific and strongly down-regulated in an1, ph3 and ph4 mutants, while it is unaffected in ph5 and ph2 mutants.

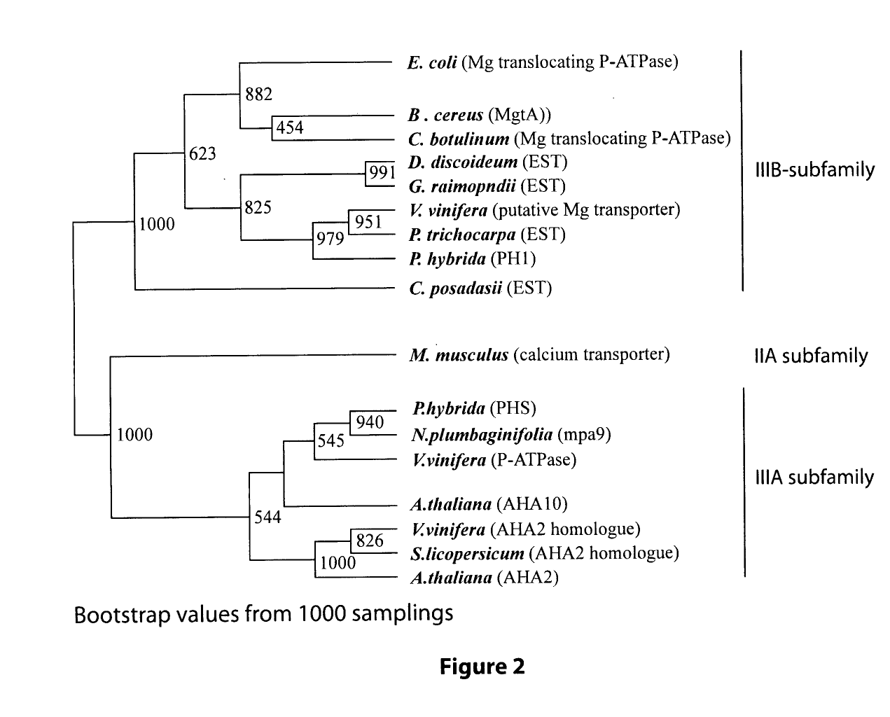

The predicted protein encoded by the PH1 gene is a P_{3B}ATPase has very high homology to a family of Mg²⁺ transporters well characterized in bacteria (Maguire, Frontiers in Bioscience 11:3149-3163, 2006). Protein BLAST search identified only one member of this family from plants (a hypothetical protein from grape) and a long list of bacterial proteins with very high homology to PH1. Nucleotide BLAST search only identified a genomic fragment from grape and a BLAST search of the translated EST collection in NCBI resulted in a few plant proteins of this class (from peach, oak, avocado, poplar, cotton, pine tree, euphorbia, orange and tangerine), a less related sequence from Ascomycetes fungi, one from Dictyostelium and a very long list of bacterial proteins. No related sequences appear to be present in animals, as the first BLAST hit is a Ca⁺ transporter from mouse which belongs to a different group of P-ATPases (FIG. 2).

Remarkably no transporters of this family are present in yeast, Arabidopsis or rice, while extremely high conservation (see FIG. 2) is observed between the petunia (and other plants) PH1 and the homologues from bacteria. This suggests that plants have acquired the PH1 protein from bacteria and then several families might have lost it again. The high level of conservation of the sequence also suggests that the function might be strongly conserved. In entero bacteria species, in comparison to the constitutively expressed CorA system for the transport of Mg²⁺, other proteins of the class to which PH1 belongs (called mgtA, mgtB and mgtC) also contribute to the control of the magnesium content in the cells. mgtA and mgtB have been shown to mediate Mg²⁺ influx with (and not against) the electrochemical gradient (Smith and Maguire, Molecular Microbiology 28:217-226, 1998, Maguire supra 2006). The transcription of these loci in bacteria, as well as the degradation of their transcript, is activated by the extracellular concentration of Mg²⁺ (Spinelli et al, FEMS microbial lett 280:226-234, 2008).

### Example 2

### Localization of Membrane PH1 Protein and Complementation of ph1 Mutant

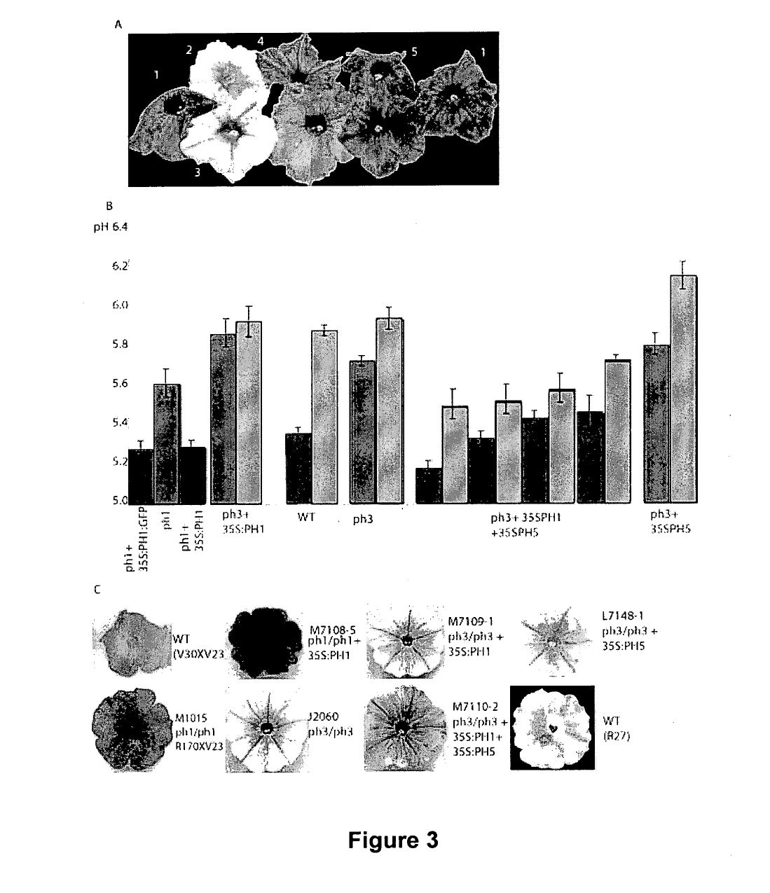

A construct was produced for the expression of a PH1:GFP fusion protein. When permanently transformed in ph1 mutant plants, this construct completely complements the mutant phenotype (FIG. 3B) demonstrating that the fusion product is active and therefore a bona fide marker for the localization of PH1. Agroinfiltration of this same construct in petals of wild-type plants resulted in a (weak) florescence signal on the tonoplast, in a pattern identical to that observed for the PH5:GFP chimeric protein (Verweij et al, 2008 supra).

The phenotype of ph1 mutant flowers is indistinguishable from that of ph5 mutants (Verweij et al, 2008 supra). Also the actual pH shift measured in the crude extract of the flowers is identical (see FIGS. 3A and 3B). The question arises at this point of how PH1 can affect acidification of the vacuolar lumen by transporting cations. The active transport of protons towards the lumen of the vacuole by the activity of PH5 builds a pH gradient across the tonoplast and results in an increase of the electrochemical gradient. It is conceivable that the activity of PH5 is quickly reduced as such gradient becomes steep and therefore the pumping of protons has to happen against a stronger contrary electrical force. The function of PH1 might be that of decreasing such electrical gradient, maintaining high activity of PH5 and making it possible to reach a relatively high concentration of protons in the vacuole.

Petunia ph4, ph3 and an1 mutant flowers do not express PH5 and PH1, therefore the question was put forward whether other PH3-PH4-AN1 controlled factors were required for vacuole acidification in petal epidermis.

Both Petunia PH5 and Petunia PH1 were constitutively express in ph3, ph4 and an1 petunia mutants using the CaMV35S promoter. As shown in FIGS. 3B and 3C, transgenic plants (of ph3 background) with high expression of both transgenes showed wild-type phenotype (reddish flowers) and a pH value from crude flower extract comparable to the pH of wild-type flowers. Plants with lower expression of the transgenes showed intermediate phenotype and intermediate pH value of the crude petal extract. Transformants with an1 and ph4 mutant backgrounds are now being produced to test the hypothesis that the combination of PH5 and PH1 can complement the ph mutant phenotype in these lines. The results described demonstrate that no other protein, whose expression is under the control of PH3, PH4 and AN1, is required to achieve acidification of the compartment where the anthocyanins are accumulated. Reference to “petunia” means Petunia hybrida.

Interestingly, these same transgenic plants showed strong acidification of the crude extract of the leaves (FIG. 3B). In agroinfiltration experiments of leaves with GFP tagged PH1 or PH5, both proteins could be shown to localize on the tonoplast in leaf tissue. Therefore it is concluded that PH5 and PH1 together can acidify the vacuolar lumen of cell types other than those specialized for pigment display and their activity does not require other, petal specific, factors.

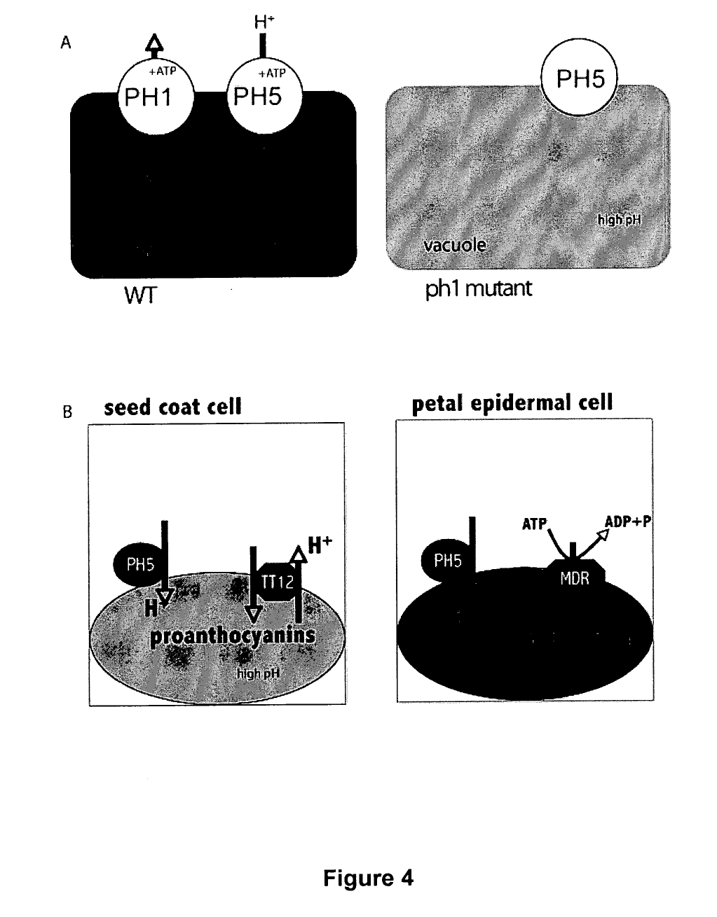

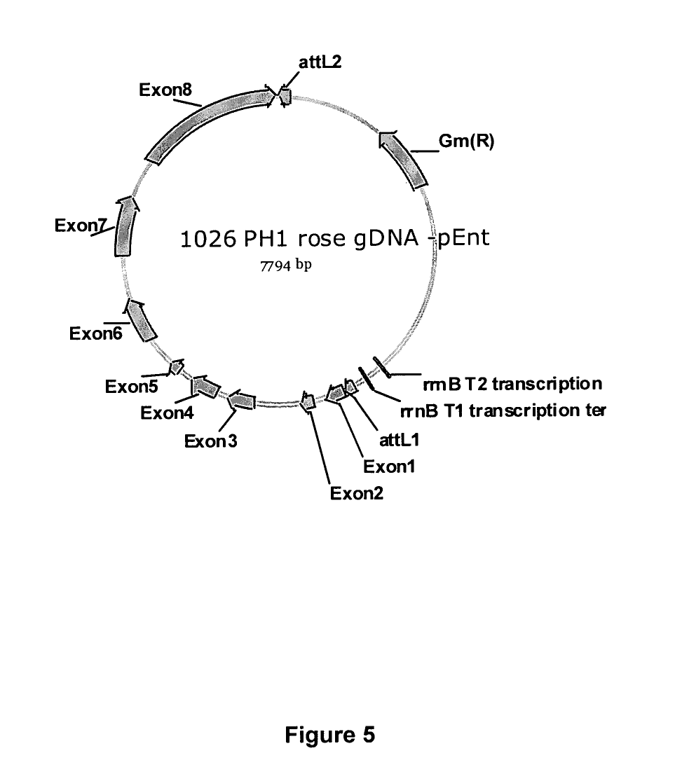

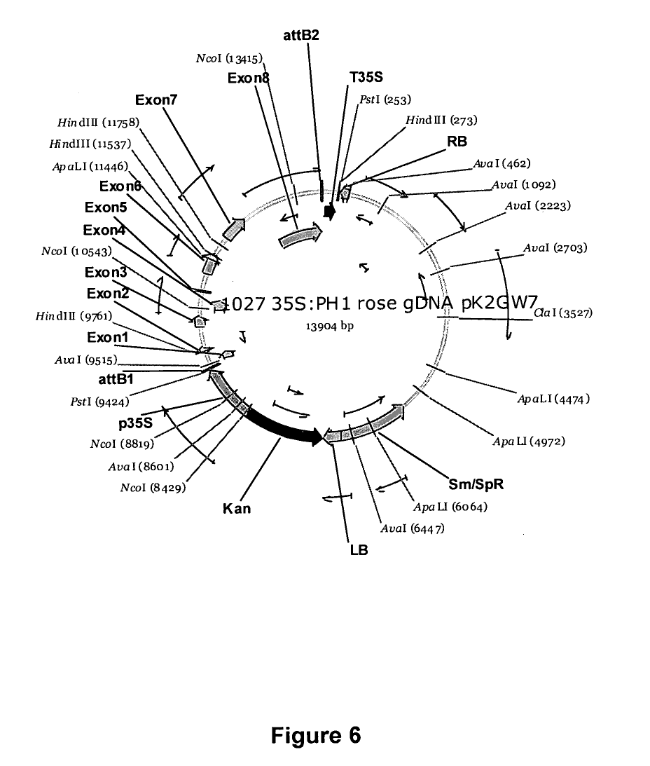

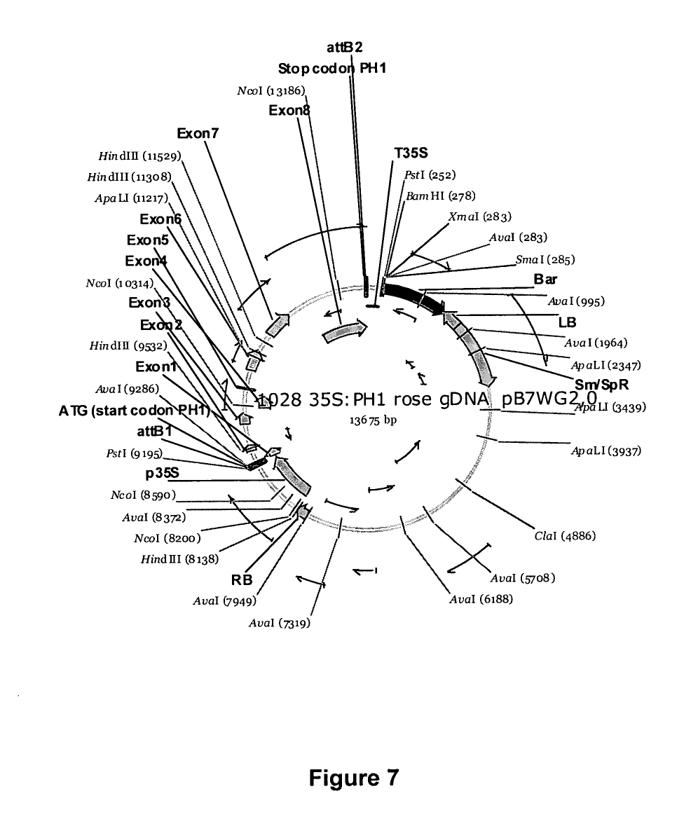

In FIG. 4 a model is proposed for the concerted action of PH5, PH1 and other proteins on endomembranes and of their effect on the lumen content. In seed coat cells, the activity of PH5 on the tonoplast of the central vacuole is required to build a pH gradient which is then used by a MATE type transporter (Debeauj on et al, Plant Cell 13:853-871, 2001) to accumulate proanthocyanins in the lumen. On this membrane, PH5 does not have to pump protons against a growing electrochemical gradient as the MATE protein uses the H⁺ gradient to transport the pigment molecules (FIG. 1A). PH1 activity is not required in these cells (Arabidopsis does not have a PH1 gene although the activity of the PH5 homolog AHA10 is required to color the seeds and petunia ph1 mutants have a normal colored coat).

PH1 activity became necessary when plants started coloring flowers (or fruits, like in the case of grape) to attract pollinators (or other animals for seed dispersal). In petal epidermal cells, the protein that transports anthocyanin molecules into the central vacuole does not require a pH gradient across the tonoplast (as shown by the fact that ph mutants accumulate the same pigments as the corresponding wild-type). This strongly suggests that the transporter in question might belong to the ABC family that uses ATP as a driving force. Nevertheless, in order to display the right color and to efficiently stabilize the pigment into the vacuolar lumen, petals need acidic vacuoles. As the anthocyanin transporter does not normalise the proton gradient thereby allowing introduction of pigments into the vacuole (as it is dependent on ATP), the action of PH5 can result in a high concentration of H⁺ in the vacuolar lumen, provided that the electrochemical gradient is kept low by the action of PH1. This could explain why certain species that do not display colored petals (e.g. Arabidopsis) have lost this (originally bacterial) protein and might mean that PH1 is part of the rather modern (in an evolutionary scale) adaptation of cells to accumulate and display anthocyanins.

### Example 3

### Isolation of a PH1 Sequence from Rose

For the isolation of the rose PH1 gene, degenerate primers (SEQ ID NO:5-23) were designed from aligned sequences of PH1 cDNA sequences of Petunia hybrida (SEQ ID NO:3) and P-ATPase sequences from Vitus vinifera (partial sequence) and Gossypium raimondii (partial sequence). A touchdown PCR from 65-58° C. was performed on gDNA with 24 combinations of these primers. This resulted in the successful amplification of two overlapping PCR products using primers SEQ ID NO:13 and SEQ ID NO:14 (272 by fragment) and SEQ ID NO:13 and SEQ ID NO:15 (772 by fragment). Sequence specific primers were designed from sequences generated from these PCR fragments. The primers were used to amplify the complete cDNA, including the 5′ and the 3′ UTR (untranslated region), from rose PH1 using First Choice 5′ RLM-RACE kit (Ambion, USA). It was not possible to obtain the full sequence in one step because the PCR fragments were far downstream of the 5′UTR. The full size cDNA was thus obtained using combinations of specific and degenerated primers, resulting in the 3083 by cDNA (SEQ ID NO:1) and 4675 by genomic rose PH1 DNA fragment.

### Example 4

### Isolation of PH1 Sequence from Other Species

For the isolation of the PH1 gene from other plants degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

### Example 5

### PH1 Genes from Grape and Rose

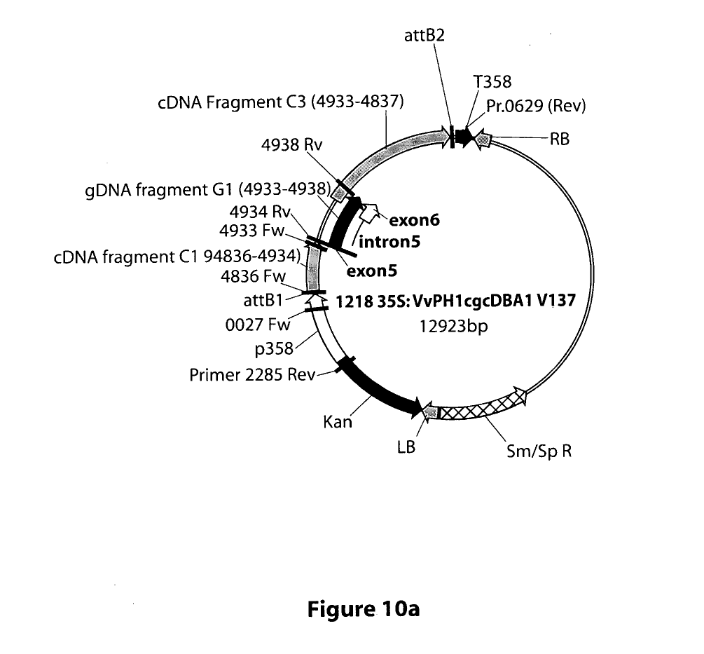

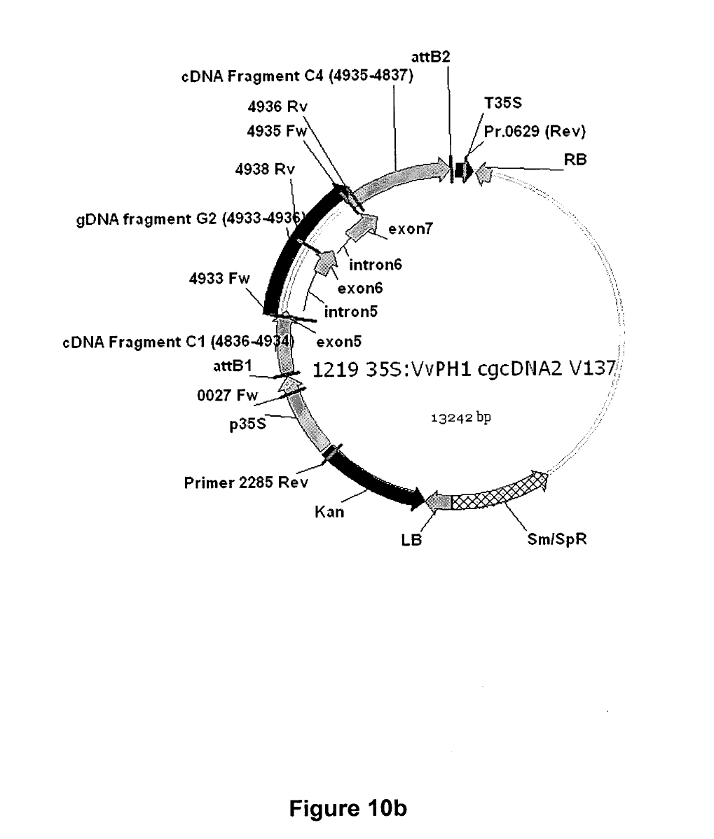

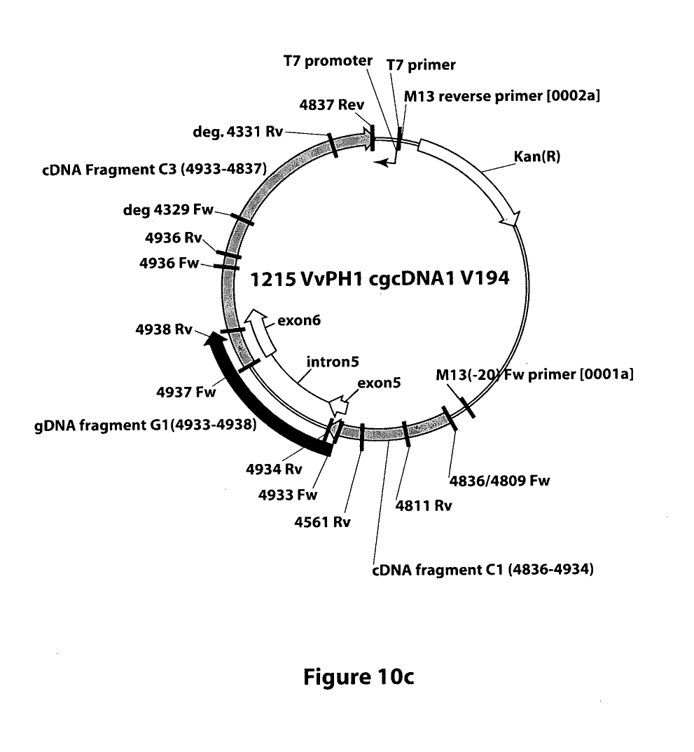

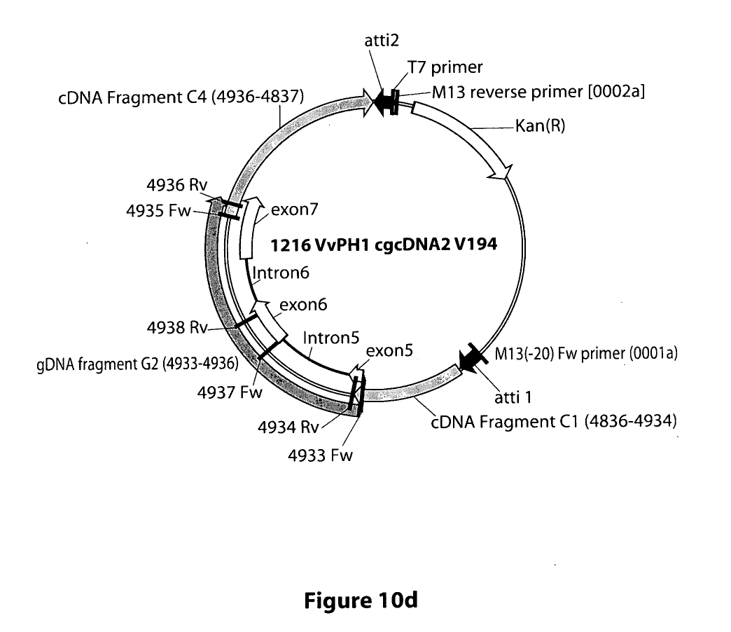

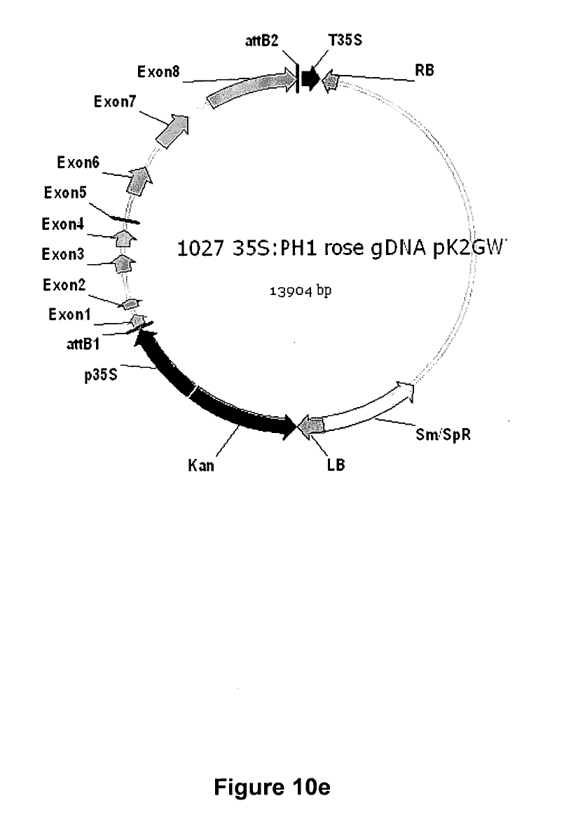

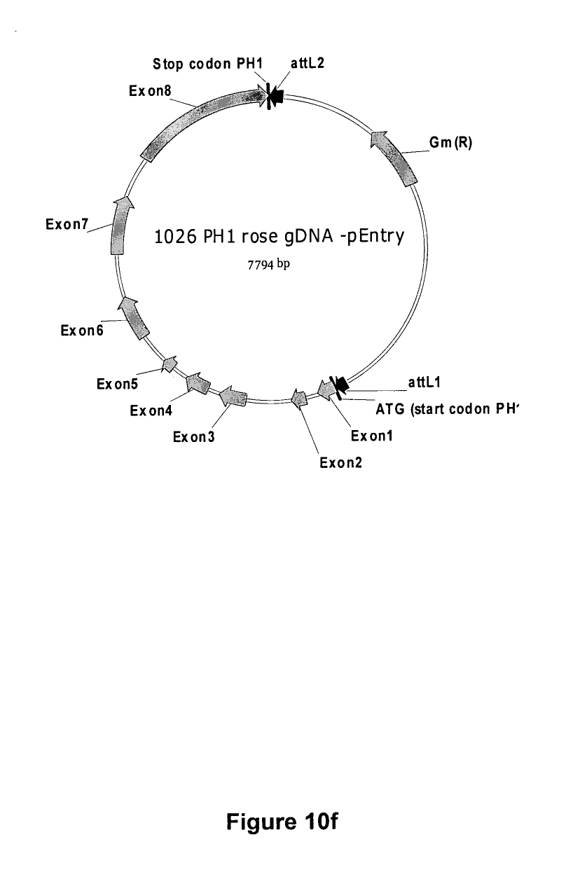

PH1 homologs have been identified from rose and grape and 35S expression constructs prepared both genes. The isolation of the PH1 gene from grape (Vitis vinifera) was totally done in silico by blasting the PH1 sequence from petunia against the grape genome sequence. With primers designed on the basis of this sequence, the genomic and cDNA sequences where amplified from cultivar (cv) Nebbiolo. Due to grape cultivars often being heterozygous, the cloning of PH1 sequences from the cv Nebbiolo has resulted in two different coding sequences and these have been used in the experiments aiming to the complementation of the petunia ph1 mutant. The expression constructs for the PH1 gene from grape are construct number 1218 (FIG. 10a) and 1219 (FIG. 10b).

Primers used to produce these contracts:

4836(+ attB1)
(SEQ ID NO: 48)
GGGGACAAGTTTGTACAAAAAAGCAGGCTTTATGGCAACTCCCAGATTTT
4934
(SEQ ID NO: 49)
TCT AGC AAA GGA GTG CTC TGA TCT
4933
(SEQ ID NO: 50)
CAC TAA CAG GGG AGT CTG GAG T
4936
(SEQ ID NO: 51)
ATC TTC TAG GGA GAA AGT TGT GAT TG
4935
(SEQ ID NO: 52)
TCA CTC GAG AGG TTT GTG GTA AC
4837(+ attB2)
(SEQ ID NO: 53)
GGG GAC CAC TTT GTA CAA GAA AGC TGG GT A TTA CAG
CCA TTT GTG GTA GA

The transformation in petunia ph1 mutants of both constructs for the expression of grape PH1 (constructs 1218 and 1219 [FIGS. 10a and 10b]) results in full complementation of the phenotype, demonstrating that these are the true homologs of the petunia PH1 gene. Construct 1304 was made for the expression of PH1 gene of rose (FIGS. 10e and 10f).

Primers used to make the rosePH1 entry clone:

4446 (PH1 rose ATG + attB1 F)
(SEQ ID NO: 54)
GGGGACAAGTTTGTACAAAAAAGCAGGCTATGAGAACTTTCAAAATCCC
CACCA
4447 (PH1 rose stop + attB2 R)
(SEQ ID NO: 55)
GGGGACCACTTTGTACAAGAAAGCTGGGTTCATTCTGCTACCTAAAGCC
AGGTT

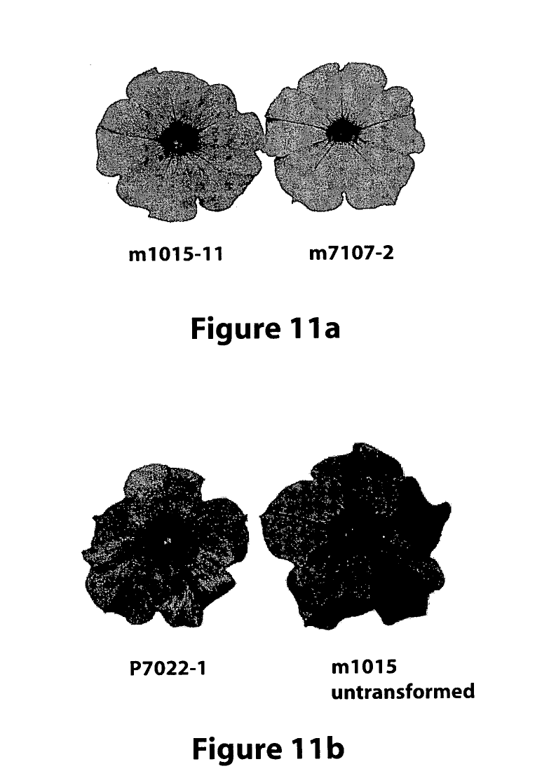

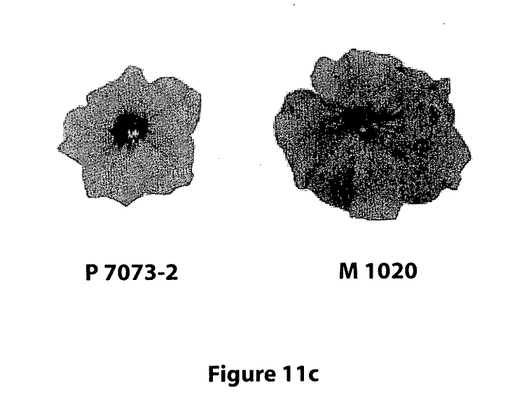

The rose PH1 gene fully complemented the petunia phi mutant. See FIG. 11b and Table 3. The same full complementation is the result of the expression of the PH1 gene from grape, see FIG. 11c and Table 3.

Values of pH of the crude flower extract in transgenics (=expressor) expressing the rosePH1 gene are shown in Table 3 (for all experiments at least four flowers of the same plant have been sampled).

TABLE 3
pH flower crude extract

| Plant | pH |
|---|---|
| untransformed ph1 mutant N1 | 5.60 (±0.05) |
| untransformed ph1 mutant N2 | 5.55 (±0.02) |
| untransformed ph1 mutant N3 | 5.55 (±0.05) |
| P7022 N1 | 5.25 (±0.05) |
| P7022 N2 | 5.30 (±0.01) |
| P7022 N3 | 5.25 (±0.05) |
| P7079 N1 | 5.35 (±0.1) |
| P7079 N2 | 5.15 (±0.15) |

(P7022 = transgenic petunia plants expressing rose PH1, N1, N2 and N3 indicate different independent transgenic plants. P7079 = transgenic petunia plants expressing grape PH1, N1, N2 and N3 indicate different independent transgenic plants)

These experiments showed that the whole pathway of vacuolar acidification in petunia petals is present also in other species that accumulate anthocyanins in petals or in fruits and represent a good experimental basis for the design and test of constructs aiming to produce flowers with high vacuolar pH in commercially valuable species.

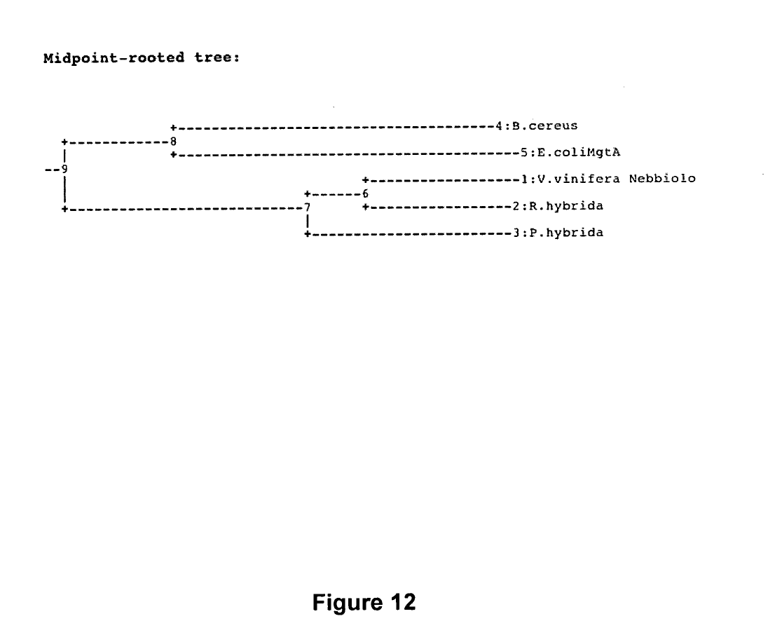

Phylogenetic tree resulting from the alignment of full size PH1 homolog proteins from different species is shown in FIGS. 1, 2 and 12.

B. cereus=Bacillus cereus
E. coli MgtA=MgtA protein from Eschericchia coli
V. vinifera Nebbiolo=Vitis vinifera cultivar Nebbiolo
R. hybrida=Rosa hybrida=RH
P. hybrida=Petunia hybrida=PH

### Example 6

### Down Regulation of Rose PH1 in Rose

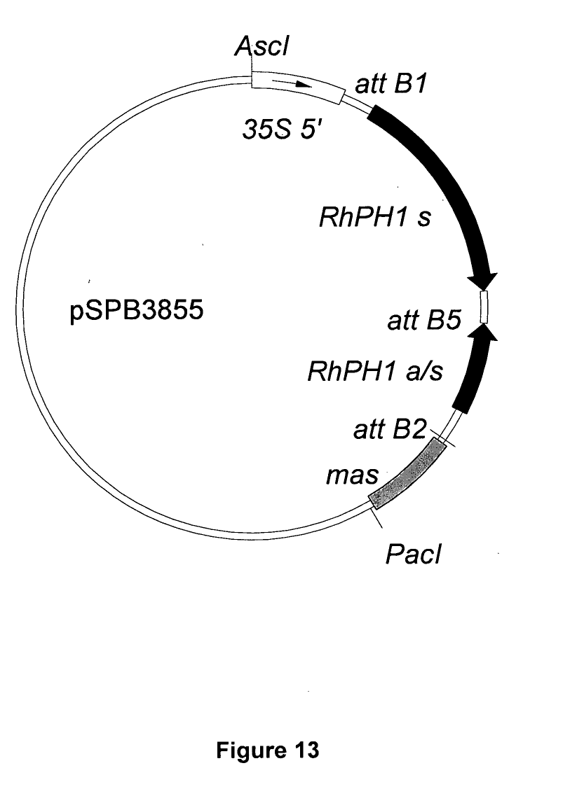

An expression cassette containing an enhanced 35S promoter (e35S) [Mitsuhara et al, Plant Cell Physiol 37:49-59, 1996], a rose PH1 fragment (from nucleotide 202 to nucleotide 921 of SEQ ID NO:1) in sense orientation, a rose PH1 fragment (from nucleotide 301 to nucleotide 600 of SEQ ID NO:1) in reverse orientation and a mas terminator (terminator fragment from the mannopine synthase gene of Agrobacterium) was constructed using the Gateway system (Invitrogen) and protocols were followed according to the manufacturer's instruction. The resulting plasmid vector was designated as pSPB 3855 (FIG. 13). A binary vector for transcription of double-stranded RNA from rose PH1 is constructed in a backbone of pBin Plus (van Engelen, Transgenic Research 4:288-290, 1995).

Rosa hybrida cv. Lavande is transformed with Agrobacterium tumefaciens AGL0 harbouring the transformation vector containing the expression cassette from pSPB3855. Rose transformation is performed according to procedures in Katsumoto et al, Plant Cell Physiol. 48:1589-1600, 2007. Transgenic plantlets are selected on kanamycin. Plantlets are sent to soil and flowered. Flowers are examined for change in color and pH of crude petal extracts are analyzed.

### Example 7

### The Expression of Petunia PH5 and Petunia PH1 Acidfies the Vacuolar Lumen

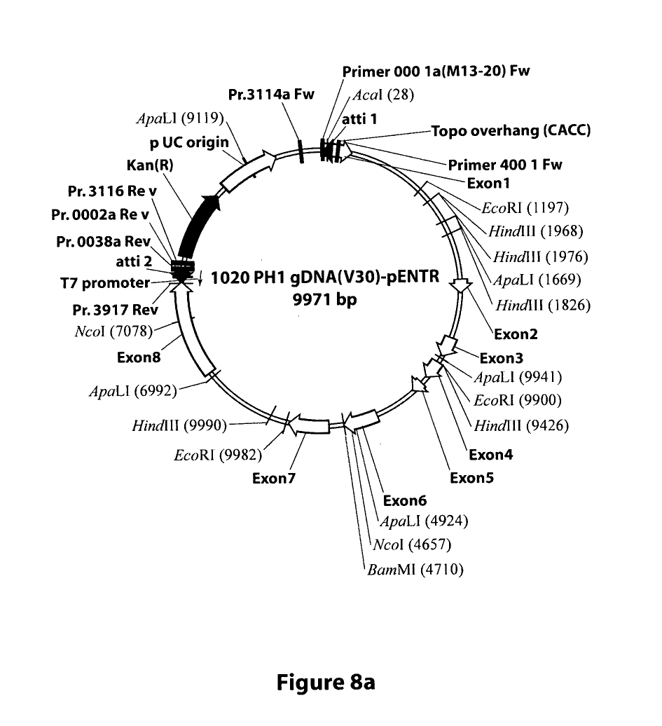

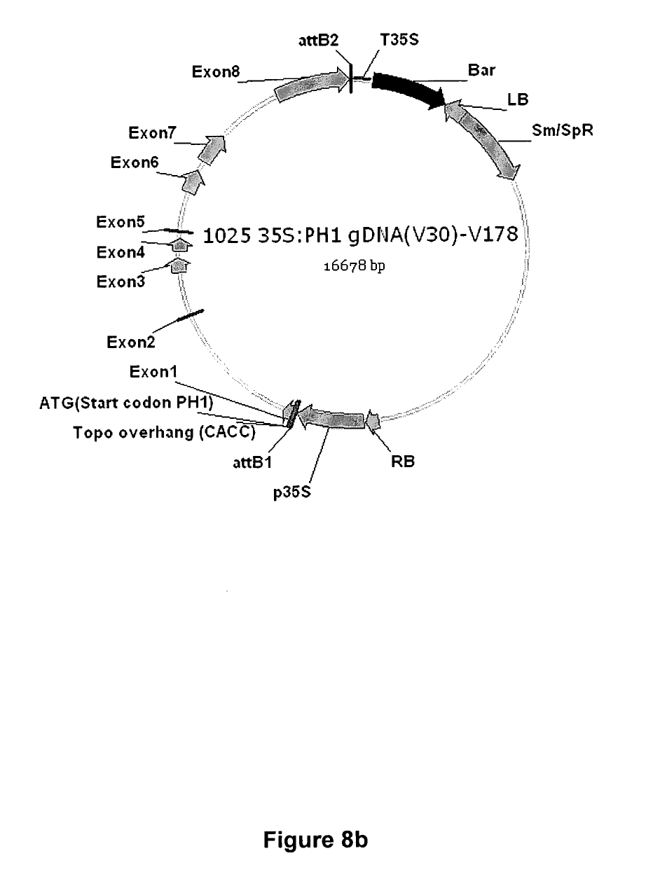

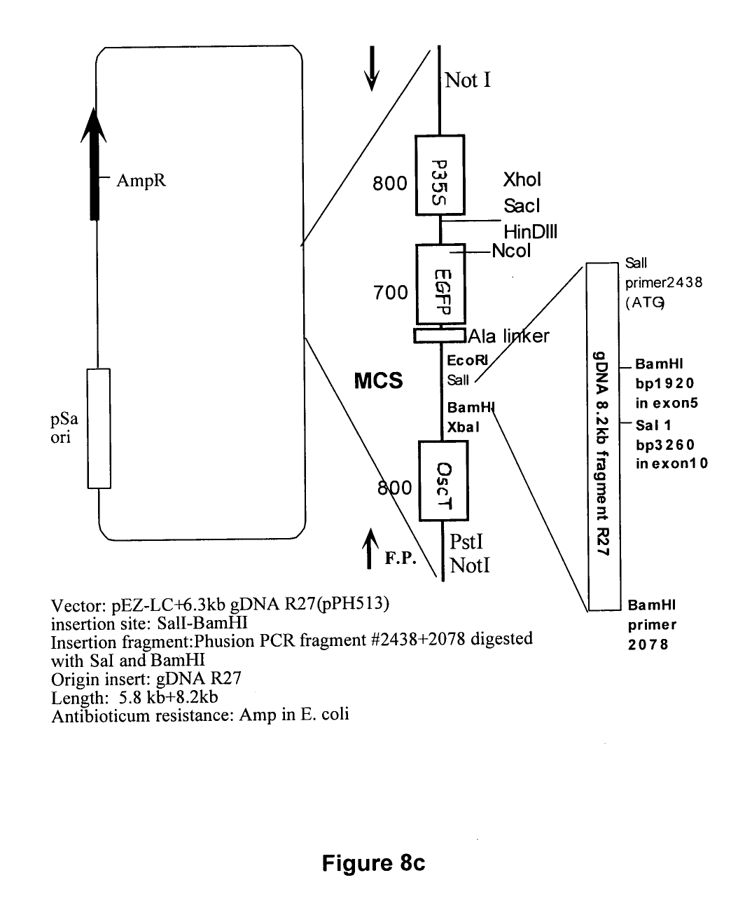

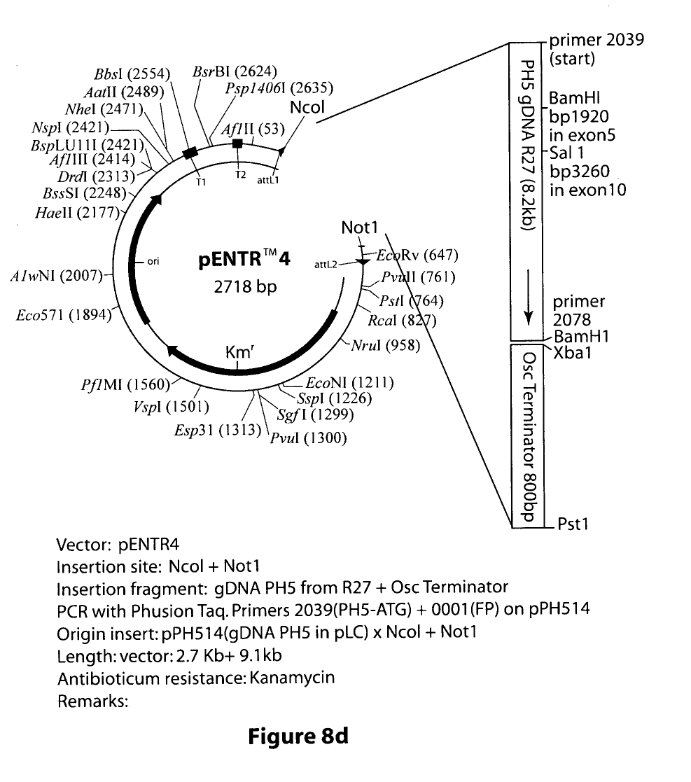

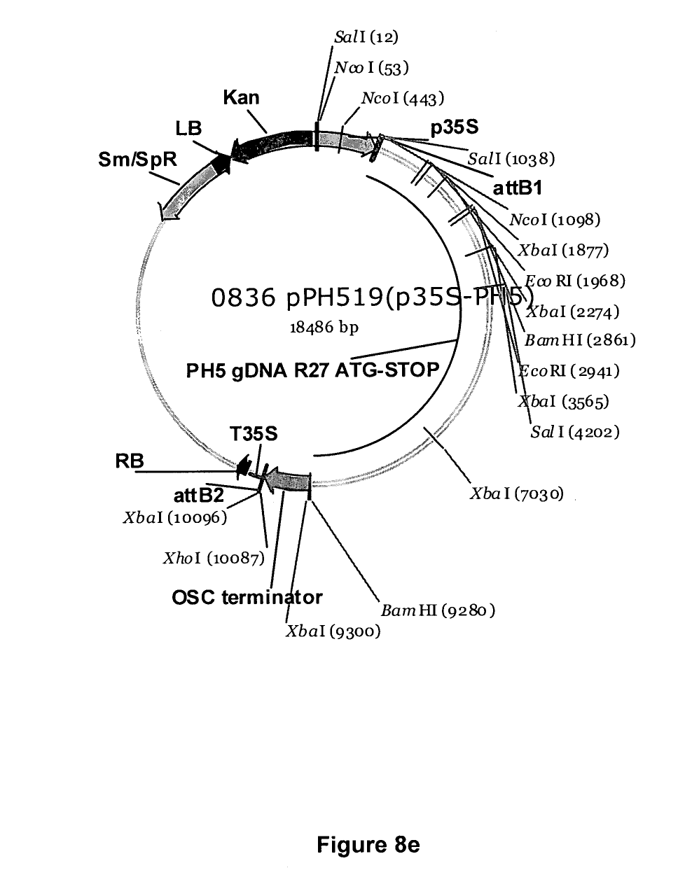

A reconstruction experiment was conducted to establish which of the target genes of the pH regulators AN1, PH3 and PH4 are required for the proton pumping activity of PH5. A ph3 mutant (J2060) was transformed with a 35S promoter driven PH5 and a 35S promoter driven petunia PH5 and a 35S promoter driven petunia PH1. The 35S:PH1 (construct number 1025 [FIG. 8b]) construct was obtained as follow: the genomic fragment containing the PH1 coding sequence (from ATG to STOP) and all intron sequences, was amplified as PCR fragment from petunia genomic DNA (line V30) using Phusion polymerase with primers 4001 (CACCATGTGGTTATCCAATATTTTCCCTGT—SEQ ID NO:56) and 3917 (TAGGACTAAAGCCATGTCTTGAA—SEQ ID NO:57) and cloned by TOPO isomerase reaction in the entry vector pENTR/D-TOPO to give construct 1020 (FIG. 8a). Constructs are shown in FIGS. 8a through 8e.

The 35S:PH5 construct (construct 893—FIG. 8c) contains the PH5 genomic fragment (from ATG to STOP, including introns) under the 35SCaMV promoter and the OCS terminator (terminator fragment for octopine synthase gene of Agrobacterium) in the vector pK2GW7,0. This was obtained by LR reaction from the entry clone 835 (FIG. 8d).

The entry clone was made by cutting the PH5 gDNA fragment (from lineR27) and the OCS terminator cloned in pENTR4 with NcoI and NotI. The gDNA fragment containing petunia PH5 in this clone originates from clone 831 (FIG. 8c). The PH5 gene is disclosed in Verweij et al, 2008 supra and in International Patent Application Nos. PCT/AU2006/000451 and PCT/AU2007/000739, the entire contents of which are incorporated by reference. In this construct the genomic fragment of PH5 was obtained by Phusion PCR with primers 2438 (CCTATTCATCGTCGACACATGGCCGAAGATCTGGAGAGA—SEQ ID NO:46) and 2078 (CGGGATCCTGGAGCCAGAAGTTTGTTATAGGAGG—SEQ ID NO:47) from genomic DNA of petunia line R27. The fragment was inserted in SalI/BamHI site of pEZ-LC.

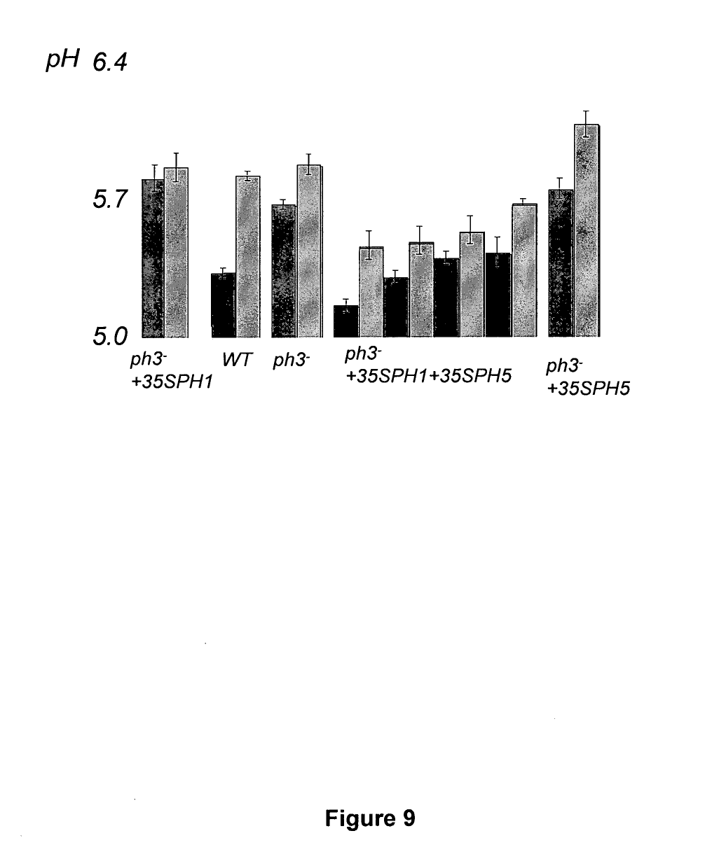

The regenerants showing relatively high expression of both transgenes (still within the wild-type level of expression of the endogenous genes) harbored fully red flowers (wild-type phenotype) and the pH of the crude flower extracts was similar to that of wild-types in the same genetic background (cyanidin accumulating line in which the ph3 mutation is due to a transposon insertion in the PH3 gene). Surprisingly, the pH of the crude extracts of the leaves of these transgenics was lower than that of the wild-type and the untransformed controls (FIG. 9).

ph4 and an1 mutants were transformed with 35S:PH5 and 35S:PH1 constructs (using the very same construct described above for the transformation in ph3 mutants). ph4 mutants were not generated in any plant in which the color phenotype was restored. Nevertheless, the pH of the flower extract was strongly diminished in comparison to the untransformed ph4 mutant. The difference in pH was in some plants half a pH unit. This pH shift was not sufficient to shift the color (maybe due to the low expression of the transgenes). Nevertheless, it was demonstrated that PH5 and PH1 together can acidify the vacuole of ph4 mutant flowers.

The transformants in an1 mutant background also showed a strong difference in pH of the flower extract (half a pH unit or more). In this case the absence of anthocyanins makes it impossible to evaluate whether this shift would be sufficient for a color difference (Table 4).

In leaves of only a few ph4 and an1 mutants expressing PH5 and PH1 a much less relevant acidification of the crude extract could be detected.

TABLE 4
Values of pH of the crude extract of flowers and leaves of
transgenic plants and controls (for each value n > 4)

| | pH flower | pH leaf |
|---|---|---|
| an1 mutant + 35S:PH1 | 5.7 (±0.2) | 5.65 (±0.15) |
| an1 mutant + 35S:PH5 | 5.65 (±0.15) | 5.6 (±0.22) |
| an1 mutant 35S:PH1 + | 5.25 (±0.14) | 5.2 (±0.13) |
| 35S:PH5 | | |
| an1 mutant | 5.7 (±0.16) | 5.65 (±0.13) |
| ph4 mutant | 5.9 (±0.24) | 5.9 (±0.38) |
| PH4 Revertant | 5.4 (±0.14) | 5.9 (±0.11) |
| ph4 mutant + 35S:PH1 + | 5.6* (±0.22) | 5.8* (±0.3) |
| 35S:PH5 | | |

*only in the strongest expressors

All together these results demonstrate that petunia PH5 and petunia PH1 can drive vacuolar acidification in petal epidermal cells independently from other factors controlled by the transcription factors AN1, PH3 and PH4. The observation that in plants with high expression of PH1 and PH5 also in leaves, the vacuoles are acidified in these tissue as well, suggests that these two transporters are sufficient to obtain acid vacuoles also in tissues other then petals (where PH4, AN1 and PH3 are normally not expressed). The minimal unit able to acidify the vacuole of any cell type in the plant has been identified. It is proposed to check more tissues and to try the effect of the combined expression of these two proteins also in other plant species and even other organisms

### Example 8

### Isolation of a PH1 Sequence from Dianthus spp

For the isolation of the carnation PH1 gene, degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

### Example 9

### Isolation of PH1 Sequence from Gerbera spp

For the isolation of the gerbera PH1 gene, degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

### Example 10

### Isolation of PH1 Sequence from Chrysanthemum spp

For the isolation of the chrysanthemum PH1 gene, degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

### Example 11

### Isolation of PH1 Sequence from Denderanthema spp

For the isolation of the denderanthema PH1 gene, degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

### Example 12

### Isolation of PH1 Sequence from Lily

For the isolation of the lily PH1 gene, degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

### Example 13

### Isolation of PH1 Sequence from Gysophila spp

For the isolation of the gysophila PH1 gene, degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

### Example 14

### Isolation of PH1 Sequence from Torenia spp

For the isolation of the torenia PH1 gene, degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

### Example 15

### Isolation of PH1 sequence from Orchid

For the isolation of the orchid PH1 gene, degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

### Example 16

### Isolation of PH1 Sequence from Cymbidium spp

For the isolation of the cymbidium PH1 gene, degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

### Example 17

### Isolation of PH1 Sequence from Dendrobium spp

For the isolation of the dendrobium PH1 gene, degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

### Example 18

### Isolation of PH1 Sequence from Phalaenopsis spp

For the isolation of the phalaneopsis PH1 gene, degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

### Example 19

### Isolation of PH1 Sequence from Cyclamen spp

For the isolation of the cyclamen PH1 gene, degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

### Example 20

### Isolation of PH1 Sequence from Begonia spp

For the isolation of the begonia PH1 gene, degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

### Example 21

### Isolation of PH1 Sequence from Iris spp

For the isolation of the iris PH1 gene, degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

### Example 22

### Isolation of PH1 Sequence from Alstroemeria spp

For the isolation of the alstroemeria PH1 gene, degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

### Example 23

### Isolation of PH1 Sequence from Anthurium spp

For the isolation of the anthurium PH1 gene, degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

### Example 24

### Isolation of PH1 Sequence from Catharanthus spp

For the isolation of the catharanthus PH1 gene, degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

### Example 25

### Isolation of PH1 Sequence from Dracaena spp

For the isolation of the dracaena PH1 gene, degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

### Example 26

### Isolation of PH1 Sequence from Erica spp

For the isolation of the erica PH1 gene, degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

### Example 27

### Isolation of PH1 Sequence from Ficus spp

For the isolation of the ficus PH1 gene, degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

### Example 28

### Isolation of PH1 Sequence from Freesia spp

For the isolation of the freesia PH1 gene, degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

### Example 29

### Isolation of PH1 Sequence from Fuchsia spp

For the isolation of the fuchsia PH1 gene, degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

### Example 30

### Isolation of PH1 Sequence from Geranium spp

For the isolation of the geranium PH1 gene, degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

### Example 31

### Isolation of PH1 Sequence from Gladiolus spp

For the isolation of the gladiolus PH1 gene, degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

### Example 32

### Isolation of PH1 Sequence from Helianthus spp

For the isolation of the helianthus PH1 gene, degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

### Example 33

### Isolation of PH1 Sequence from Hyacinth spp

For the isolation of the hyacinth PH1 gene, degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

### Example 34

### Isolation of PH1 Sequence from Hypericum spp

For the isolation of the hypericum PH1 gene, degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

### Example 35

Isolation of PH1 sequence from Impatiens spp

For the isolation of the impatiens PH1 gene, degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

### Example 36

### Isolation of PH1 Sequence from Iris spp

For the isolation of the iris PH1 gene, degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

### Example 37

### Isolation of PH1 Sequence from Chamelaucium spp

For the isolation of the chamelaucium PH1 gene, degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

### Example 38

### Isolation of PH1 Sequence from Kalanchoe spp

For the isolation of the kalanchoe PH1 gene, degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

### Example 39

### Isolation of PH1 Sequence from Lisianthus spp

For the isolation of the lisianthus PH1 gene, degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

### Example 40

### Isolation of PH1 Sequence from Lobelia spp

For the isolation of the lobelia PH1 gene, degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

### Example 41

### Isolation of PH1 Sequence from Narcissus spp

For the isolation of the narcissus PH1 gene, degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

### Example 42

### Isolation of PH1 Sequence from Nierembergia spp

For the isolation of the nierembergia PH1 gene, degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

### Example 43

### Isolation of PH1 Sequence from Ornithoglaum spp

For the isolation of the ornithoglaum PH1 gene, degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

### Example 44

### Isolation of PH1 Sequence from Osteospermum spp

For the isolation of the osteospermum PH1 gene, degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

### Example 45

### Isolation of PH1 Sequence from Paeonia spp

For the isolation of the paeonia PH1 gene, degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

### Example 46

### Isolation of PH1 Sequence from Pelargonium spp

For the isolation of the pelargonium PH1 gene, degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

### Example 47

### Isolation of PH1 Sequence from Plumbago spp

For the isolation of the plumbago PH1 gene, degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

### Example 48

### Isolation of PH1 Sequence from Primrose spp

For the isolation of the primrose PH1 gene, degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

### Example 49

### Isolation of PH1 Sequence from Ruscus spp

For the isolation of the ruscus PH1 gene, degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

### Example 50

### Isolation of PH1 Sequence from Saintpaulia spp

For the isolation of the saintpaulia PH1 gene, degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

### Example 51

### Isolation of PH1 Sequence from Solidago spp

For the isolation of the solidago PH1 gene, degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

### Example 52

### Isolation of PH1 Sequence from Spathiplyllum spp

For the isolation of the spathiplyllum PH1 gene, degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

### Example 53

### Isolation of PH1 Sequence from Tulip spp

For the isolation of the tulip PH1 gene, degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

### Example 54

### Isolation of PH1 Sequence from Verbena spp

For the isolation of the verbena PH1 gene, degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

### Example 55

### Isolation of PH1 sequence from Viola spp

For the isolation of the viola PH1 gene, degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

### Example 56

### Isolation of PH1 Sequence from Zantedeschia spp

For the isolation of the zantedeschia PH1 gene, degenerate primers are designed from aligned sequences of PH1 cDNA sequences of Petuna hydrida and P.ATPase sequences from Vitus vinifera and Gossypium raimondii. Alignments with other PH1 sequences may also be conducted. Cloning is generally via PCR amplification and screening. A single or multiple steps may be required.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Altschul et al, Nucl. Acids Res. 25: 3389-3402, 1997
Altschul et al, J. Mol. Biol. 215(3): 403-410, 1990
Ausubel et al, “Current Protocols in Molecular Biology” John Wiley & Sons Inc, 1994-1998, Chapter 15.
Bonner and Laskey, Eur. J. Biochem. 46: 83, 1974
Brugliera et al, Plant J. 5, 81-92, 1994
de Vetten et al, Genes Dev. 11:1422-1434, 1997
de Vetten et al, Plant Cell 11(8):1433-1444, 1999
Debeaujon et al, Plant Cell 13:853-871, 2001
Frohman et al, PNAS 85: 8998-9002, 1988
Holton et al, Nature 366:276-279, 1993
Holton and Cornish, Plant Cell 7:1071-1083, 1995
Huang and Miller, Adv. Appl. Math. 12: 373-381, 1991
Gamborg et al., Experimental Cell Research, 95:355-358, 1970
Katsumoto et al, Plant Cell Physiol. 48:1589-1600, 2007
Koes et al, Trends in Plant Science, May 2005
Maguire, Frontiers in Bioscience 11:3149-3163, 2006
Merrifield, J. Am. Chem. Soc. 85:2149, 1964
Mitsuhara et al, Plant Cell Physiol 37:49-59, 1996
Mol et al, Trends Plant Sci. 3: 212-217, 1998
Murashige and Skoog, Physiologia Plantarum 15:473-497, 1962
Pearson and Lipman, Proc. Natl. Acad. Sci. USA 85(8): 2444-2448, 1988
Plant Molecular Biology Labfax, Croy (ed), Bios scientific Publishers, Oxford, UK, 1993
Plant Molecular Biology Manual (2^{nd} edition), Gelvin and Schilperoot (eds), Kluwer Academic Publisher, The Netherlands, 1994
Quattrocchio et al, Plant J. 13, 475-488, 1998
Sambrook et al, Molecular Cloning: A Laboratory Manual, 2^{nd} edition, Cold Spring Harbor Laboratories, Cold Spring Harbor, N.Y., USA, 1989
Sambrook and Russell, Molecular Cloning: A Laboratory Manual 3^{rd} edition, Cold Spring Harbor Laboratories, Cold Spring Harbor, N.Y., USA, 2001
Smith and Maguire, Molecular Microbiology 28:217-226, 1998
Spinelli et al, FEMS microbial lett 280:226-234, 2008
Tanaka et al, Plant Cell, Tissue and Organ Culture 80:1-24, 2005
Thompson et al, Nucleic Acids Research 22: 4673-4680, 1994
van Engelen, Transgenic Research 4:288-290, 1995
van Houwelingen et al, Plant J. 13(1): 39-50, 1998
Verweij, In Developmental Genetics (Amsterdam: Vrije Universiteit), 2007
Verweij et al, Nature Cell Biology 10:1456-1462, 2008
Winkel-Shirley, Plant Physiol. 126:485-493, 2001a
Winkel-Shirley, Plant Physiol. 127:1399-1404, 2001b

1. An isolated PH1 or PH1 homolog from a plant which:
(i) comprises a nucleotide sequence which has at least 50% identity to SEQ ID NOs:1, 3, 42, 44, 58 or 59 after optimal alignment;
(ii) comprises a nucleotide sequence which is capable of hybridizing to SEQ ID NOs:1, 3, 42, 44, 58 or 59 or its complement;
(iii) encodes an amino acid sequence which has at least 50% similarity to SEQ ID NOs:2, 4, 43 or 45 after optimal alignment;
(iv) when expressed in a plant cell or organelle, leads to acidic conditions or when its expression is reduced in a plant cell or organelle, leads to alkaline conditions.

2. The isolated nucleic acid molecule of claim 1 wherein the molecule can complement a PH1 mutant in petunia.

3. The isolated nucleic acid molecule of claim 1 comprising the nucleotide sequence selected from in SEQ ID NO:1, 3, 42, 44, 58 and 59.

4. The isolated nucleic acid molecule of claim 1 encoding an amino acid sequence set forth in SEQ ID NO:2 or 4 or 43 or 45 or an amino acid sequence having at least 50% similarity thereto after optimal alignment.

5. The isolated nucleic acid molecule of claim 4 encoding the amino acid sequence selected from SEQ ID NO:2, 4, 43 and 45.

6. A genetic construct comprising a nucleic acid molecule operably linked to a promoter such that upon expression a mRNA transcript is produced which is antisense to the nucleic acid molecule of claim 1.

7. A genetic construct comprising a nucleic acid molecule operably linked to a promoter such that upon expression a mRNA transcript is produced which is sense to the nucleic acid molecule of claim 1.

8. A method for modulating the pH in a vacuole of a plant cell said method comprising introducing into said plant cell or a parent or relative of said plant cell a genetic construct comprising a nucleic acid molecule linked to a promoter such that upon expression a mRNA transcript is produced which is antisense to the nucleic acid molecule of claim 1, or comprising a nucleic acid molecule operably linked to a promoter such that upon expression a mRNA transcript is produced which is sense to the nucleic acid molecule of claim 1 and culturing the plant cell or plant comprising said cell or parent or relative of said cell under conditions to permit expression of the nucleic acid molecule in the genetic construct.

9. The method of claim 8 wherein the plant or plant cell is or is from a plant selected from the list consisting of Rosa spp, Petunia spp, Vitis spp, Dianthus spp, Chrysanthemum spp, Cyclamen spp, Iris spp, Pelargonium spp, Liparieae, Geranium spp, Saintpaulia spp, Plumbago spp, Kalanchoe spp. and gerbera.

10. The method of claim 9 wherein the plant or plant cell is from a rose, gerbera, carnation or chrysanthemum.

11. The method of claim 8 further comprising modulating levels of protein selected from PH5, F3′5′H, F3′H, DFR, MT and an ion transporter, for the purposes of altering flower color and other infloresence and/or taste or flavor of fruit including berries and other reproductive material.

12. A method for producing a plant capable of synthesizing a pH modulating or altering protein, said method comprising stably transforming a cell of a suitable plant with a nucleic acid sequence of nucleotide sequence which has at least 50% identity to SEQ ID NOs:1, 3, 42, 44, 58 or 59 after optimal alignment or which comprises a nucleotide sequence which is capable of hybridizing to SEQ ID NOs:1, 3, 42, 44, 58 or 59 or its complement, wherein stable transformation of the cell is under conditions permitting the eventual expression of said nucleic acid sequence, regenerating a transgenic plant from the cell and growing said transgenic plant for a time and under conditions sufficient to permit the expression of the nucleic acid sequence and optionally generating genetically modified progeny thereof.

13. The method of claim 12 wherein the plant or plant cell is selected from the list consisting of Rosa spp, Petunia spp, Vitis spp, Dianthus spp, Chrysanthemum spp, Cyclamen spp, Iris spp, Pelargonium spp, Liparieae, Geranium spp, Saintpaulia spp, Plumbago spp, Kalanchoe spp and gerbera.

14. The method of claim 13 wherein the plant or plant cell is a rose, gerbera, carnation or chrysanthemum.

15. A method for producing a plant with reduced indigenous or existing pH modulating or altering activity, said method comprising stably transforming a cell of a suitable plant with a nucleic acid molecule of claim 1 which is antisense or sense to a sequence encoding PH1, regenerating a transgenic plant from the cell and where necessary growing said transgenic plant under conditions sufficient to permit the expression of the nucleic acid and optionally generating genetically modified progeny thereof.

16. (canceled)

17. (canceled)

18. (canceled)

19. (canceled)

20. (canceled)

21. (canceled)

22. (canceled)

23. (canceled)

24. An isolated cell, plant or part of a genetically modified plant or progeny thereof which cell, plant or part comprises a reduced or elevated PH1 or PH1 homolog as defined in claim 1 wherein the pH in a vacuole of the cell or cells of the plant or plant parts is altered relative to a non-genetically modified plant.

25. The plant part of claim 24 selected from the listing consisting of a flower, fruit, vegetable, nut, root, stem, leaf and seed.

26. (canceled)

27. (canceled)

28. (canceled)

29. (canceled)

30. The isolated PH1 or PH1 homolog of claim 1 wherein the nucleotide sequence has greater than 90% identity to SEQ ID NO:1.

31. The isolated PH1 or PH1 homolog of claim 1 wherein the nucleotide sequence encodes an amino acid sequence having greater than 90% similarity to SEQ ID NO:2.

32. The isolated PH1 or PH1 homolog of claim 1 wherein the nucleotide sequence has greater than 99.95% identity to SEQ ID NO:42.

33. The isolated PH1 or PH1 homolog of claim 1 wherein the nucleotide sequence encodes an amino acid sequence having greater than 99.95% similarity to SEQ ID NO:43.


SEQUENCE LISTING (59 sequences)

SEQ ID NO: 1 - DNA, 3083 bp, artificial sequence
  Roseph1cDNA
aaaacatattatgtctctctttcattctctacggccattgctatttctggttctctgcgaatgagaactttcaaaatccccaccatttttacttctaaaagccacccgaggcttccttaccaaaaccccattcgccaaaatctagtagacaagcctgaaagccagaatggctccaacagggtgtttagattcttgcgcgggcttatgtccggagggaaaattgatggggggtcgaggacagaggcagaggagaagctttactcttggttatacgccttggcgcaatccgataaggatttggttttcgagtatgttcgatccaccgaaagaggattgagctttactgaagctgaaaggagattgaaggaaaatggcccgaatgttcctgttgatttctcttttcctagctggtggcattttttatggagtgctttctttcatccttttaatatcatattgatcgtcctgtctgtaatctcgtacataaccagtgacagcccaaatggatgcatcatgcttgttttggttttgataagtgtttgcctccggttctatcaggaatacggaagttcaaaagcagccatggaactttcagaatttgtaaggtgcccagtcaaagttcaaagatgtgcaggtagagttgttcagactgaattagtagtacaaattgatcaaagagatattgttcctggtgatattatcatatttgaacctggagacctttttcctggagatgtgagactattgtcttcgaaacaccttgttgtaagtcaggcctcattaacaggagagtcctggacaaccgaaaaaacagctgatatcagagagaatcaaagcactccattgctagatttaaggaatatttgcttcatgggaacaaatgtagtatcaggcagtggatctggtctagtggtttccactggatctaagacatacatgagcaccatgttttcaaacatagggaagaagaaaccaccaaatgaatttgaggacggtgttcgtcgcatatcttatgtgctggttgctgttatgctagtagtagtcaccatcatagttataactgactactctacatctcttgatctgtctgagagcatcctttttggagtgtcagttgcaagtgcacttactcctcaaatgcttcccctggtcgttaacacaagtcttgcaaaaggagcacttgctatggccagagacagatgcataatcaaaagcttgtctgcaatacgaaacatgggttctatggatatcttatgcattgacaagactggtacactcacaatgaatcgtgcgataatggttaattatctggacagctgggggttagacaaagaaaaggttttacagtttgctttcctcaactcatatttcaagaccgatcagaaatatcctttggatgatgcaattttggcacatgtatataccaatggattcaggttccaaccgtcaaaatggaagaaactagatgagattccttttgatttcataagaagaagggtatctattatcatggaaagagaagaagacacagaccctcacagttttgtgagagtcatggtgacaaaaggagctctggaagaagtaatgaaagtttgttcttgtatggagaatgttgacagtggcacaatttcacctctctctccagaacagtatcaaaggattataaatatgaccgaggaaataagcaatgagggactaagagttataggagtagcaacaaagaagctgggaaagataaggtatgagcgcaaagataatgatgatacttctgaatcagacatggttttcctcggcctcattacattctttgacccacccaaggactcagcaaagcaagctctgtggcggttggctgagaagggagtgaaagcaaaagtattaacaggtgactcactgtctctatctataagagtttgcaaggaagttggtatcagaacaactcatgtagttacggggccagagcttgagctactcgaccaggatgcctttcatgagactgttaaaacagcaacggtcttagctcgactcaccccaacgcagaaactccgagttgtgcaatccttgcaaacaattggtaaccacattgttggatttttgggagatggagtaaatgactcacttgcactggatgcagcccatgttggtatatcagttgattcaggagcatcagttgcaaaagactttgctgacattatcttactggagaaagacctgaatgtactcattgccggagttgaacacggccgactcacttttgggaacacaatgaagtacataaaaatgtcagttatagccaatctgggaagcgttctctcaattctcatagcaaccctggtgctcaagtatgagccattgacggcaaggcagcttcttacacagaacttcttgtatagtgtgggccagattgcaatcccatgggataaaatggaagatgattatgtaaaagtcccacaaagatggtcaaagaaaggtttgccgacgttcattttgtggaatggacctgtctgcactctttttgatgttactacacttctgttcctttggttctattataaggctgacaatctggaggatcttgttttcttccacactgcttggttcatcgaagggcttctcatgcagaccctaatcatccacttgatccgtacagagaaaattcctttcattcaggagtttgcctcatggcctgtgctttgttctacagttctggtttctgcaattggaatcgcaattacattcaccccgattgggaaagtgatgggatttatcaggcttccagtgtcatactttgggtttttggtagtactgtttataggatattttgttgttggccaggtggtcaagagactctacattttggtttataaaacctggctttaggtagcagaatgaatttcagatgagattcatgttagagactataaatagtgggagcacagagaaattaggagaaattttctcatttatcattgagagagtagtagatgttgactcaaagttgttacagggatcaatggcatttttgtagatacctctacttccacaatttatcggactgcaattgcaaaaaaaaaaaaaaaaaa

SEQ ID NO: 2 - protein, 939 aa, artificial sequence
  Roseph1 protein (deduced sequence)
MRTFKIPTIFTSKSHPRLPYQNPIRQNLVDKPESQNGSNRVFRFLRGLMSGGKIDGGSRTEAEEKLYSWLYALAQSDKDLVFEYVRSTERGLSFTEAERRLKENGPNVPVDFSFPSWWHFLWSAFFHPFNIILIVLSVISYITSDSPNGCIMLVLVLISVCLRFYQEYGSSKAAMELSEFVRCPVKVQRCAGRVVQTELVVQIDQRDIVPGDIIIFEPGDLFPGDVRLLSSKHLVVSQASLTGESWTTEKTADIRENQSTPLLDLRNICFMGTNVVSGSGSGLVVSTGSKTYMSTMFSNIGKKKPPNEFEDGVRRISYVLVAVMLVVVTIIVITDYSTSLDLSESILFGVSVASALTPQMLPLVVNTSLAKGALAMARDRCIIKSLSAIRNMGSMDILCIDKTGTLTMNRAIMVNYLDSWGLDKEKVLQFAFLNSYFKTDQKYPLDDAILAHVYTNGFRFQPSKWKKLDEIPFDFIRRRVSIIMEREEDTDPHSFVRVMVTKGALEEVMKVCSCMENVDSGTISPLSPEQYQRIINMTEEISNEGLRVIGVATKKLGKIRYERKDNDDTSESDMVFLGLITFFDPPKDSAKQALWRLAEKGVKAKVLTGDSLSLSIRVCKEVGIRTTHVVTGPELELLDQDAFHETVKTATVLARLTPTQKLRVVQSLQTIGNHIVGFLGDGVNDSLALDAAHVGISVDSGASVAKDFADIILLEKDLNVLIAGVEHGRLTFGNTMKYIKMSVIANLGSVLSILIATLVLKYEPLTARQLLTQNFLYSVGQIAIPWDKMEDDYVKVPQRWSKKGLPTFILWNGPVCTLFDVTTLLFLWFYYKADNLEDLVFFHTAWFIEGLLMQTLIIHLIRTEKIPFIQEFASWPVLCSTVLVSAIGIAITFTPIGKVMGFIRLPVSYFGFLVVLFIGYFVVGQVVKRLYILVYKTWL

SEQ ID NO: 3 - DNA, 2876 bp, artificial sequence
  PetuniaPH1 cDNA
catgatcaaccttgtttactgcaaattaaagtccaatattcaatgtggttacccaatattttcccagtaaatcactctaacataccttattataatatttctcaaaatcttgttcaaaaacccagtggacaaacgcaacataatgatggtcctaacacttcagtgttctttcgtttcttgcggaggttcacttctgcaaagaaaattgatggagggtcgagaactgaagaagaagagaagttgtattcttggatatatgctttggctcaatcagaaaaggacttggtgtacgagtatgttcaatccactgaaagaggcttgagctttgctgaagctgacagaagacttaaagaaacaggaccaaatattcctcttgagaatactttcccacagtggtggaatctactgtggagtgcttcattccatcctttcaacataattcttcttgtcctatcagtactctcttacattgcaagtgacaatccaaatggttgtatcatgcttatattagtcttcataagtgtctctctccgcttttaccaggaattcagcagctcaaaagcagcaatgaagcttgcagagtttgtacggtgtcctataaaggttcaaagatgtgcaggtagaattgttcaaactgaggtacaggttaaagttgatcaacgagaagttgttccaggtgatatcgtaattgttggaccgggggatcttttcccaggtgatgtgaggctactagaatcaaagcacctagttgtaagtcaatcttcactaacaggcgaatctgcaacgactgagaaaacagcttacgtaagagaagataacagcactccgttgctagatttgaagaacatttgctttatgggaacaagtgttgtatctggtagtggaaccggtctggttgtctctactggattaaagacgtacctcagcacaatcttttcaaaagtagggaagaaaagaccagcagatgattttgaaaaaggcatccgccacatatcatttgtgcttatcagcatcatgcttgttgtggtctcagtaattgtcctatctgtttactttacatcacgtgatctgagtaagaccatactgtatggaatctcagttgcaagtgcactcacccctcagatgcttcccctcattgtgaatactagtcttgcaaaaggagctcttgccatggccaaggatagatgtatagttaagagtttaactgctatacgaaatatgggatccatggatatcatatgcatagataagactggtacactcactgtggattttgcgactatggttaattacttcgatagctgggggtcaccaaatgaaacagtcctacactttgccttcttgaatgcttacttccaaagccaaaataagcatcctctggatgatgcaattatggcatatgcatacacaaatggtttcaggtttcagccttccaagtggaataagatagatgagattccttttgattttacaagaagaagagtatctgttatattggaaaccaaaattagcgccaaagacgagaaaataagtggtaacagagtgttgataacaaaaggagcactagaagatattttgagaatatgttctttcgttgagcacatagataagggtgtgattttaacttttaccaaagaagactacagaagaattagtgacctggcagaaagattaagtaatgaaggatatcgggttcttgggttagcaatgaaacaactcctaccagaagtcaaagttagcagcatgatctatgaggaggacgttgaatccagtatggtattcgttgggcttatatccttttttgatccaccaaaagactctgcaaagcaagcactatggcgcctagcagaaaagggagtaaaagctaaagtactgacaggtgatactctatctcttgcgataagaatatgcaaggaggtcggtataagaacaactcatgtcatcactggacctgaccttgagtcactagacacagattctttccatgagacagttaagaggtcaacagtttttgcccgacttacacctactcagaaactaagagtggtgcaatctttgcaaacaaagggtgatcatgttgttggtttcttaggagatggagtaaatgattcacttgcactggatgcagcaaatgtaggtatatctgttgactccggtgcctcaatggccaaagactttgctaacattatcttacttgagaaagacctcaatgttctcatagctggagttgagcaaggccggcttacatttggaaacacgatgaagtatatcaagatgtcagtgattgccaatctaggaagcataatttcactgctaattgcaacattgatatttggatttgagcctttgacaccaatgcagcttcttacacaaaacatcttgtataatcttggccaaattgcaataccatgggacaagatggaagattgttatgtgaaagtcccacagagatggtcacttaaaggtttagcaatgtttacatcatggaatggacctctttgttctgcatctgatatagcaaccctgttattccttttgctatattacaaggtttcaagattagatttcgaattttttcgttctgcttggttcgttgaaggacttctaatgcaaacgcttatcatacacctgatacggacagagaaaatcccctttattcaggaagttgcgtcatggccagttgtttgtgctactattcttatatcatccattggcattgtaattccgtacacaacaattggaaagattctagggttcacagccttaccattgtcatacttcggatttttggttgtgctcttcttaggttatttttcgtttggacaaattatcaagaaaggctacattttggtcttcaagacatggctttagtccta

SEQ ID NO: 4 - protein, 939 aa, artificial sequence
  PetuniaPH1 protein
MWLPNIFPVNHSNIPYYNISQNLVQKPSGQTQHNDGPNTSVFFRFLRRFTSAKKIDGGSRTEEEEKLYSYALAQSEKDLVYEYVQSTERGLSFAEADRRLKETGPNIPLENTFPQWWNLLWSASFHPFNIILLVLSVLSYIASDNPNGCIMLILVFISVSLRFYQEFSSSKAAMKLAEFVRCPIKVQRCAGRIVQTEVQVKVDQREVVPGDIVIVGPGDLFPGDVRLLESKHLVVSQSSLTGESATTEKTAYVREDNSPLLDLKNICFMGTSVVSGSGTGLVVSTGLKTYLSTIFSKVGKKRPADDFEKGIRHISFVLISIMLVVVSVIVLSVYFTSRDLSKTILYGISVASALTPQMLPLIVNTSLAKGALAMAKDRCIVKSLTAIRNMGSMDIICIDKTGTLTVDFATMVNYFDSWGSPNETVLHFAFLNAYFQSQNKHPLDDAIMAYAYTNGFRFQPSKWNKIDEIPFDFTRRRVSVILETKISAKDEKISGNRVLITKGALEDILRICSFVEHIDKGVILTFTKEDYRRISDLAERLSNEGYRVLGLAMKQLLPEVKVSSMIYEEDVESSMVFVGLISFFDPPKDSAKQALWRLAEKGVKAKVLTGDTLSLAIRICKEVGIRTTHVITGPDLESLDTDSFHETVKRSTVFARLTPTQKLRVVQSLQTKGDHVVGFLGDGVNDSLALDAANVGISVDSGASMAKDFANIILLEKDLNVLIAGVEQGRLTFGNTMKYIKMSVIANLGSIISLLIATLIFGFEPLTPMQLLTQNILYNLGQIAIPWDKMEDCYVKVPQRWSLKGLAMFTSWNGPLCSASDIATLLFLLLYYKVSRLDFEFFRSAWFVEGLLMQTLIIHLIRTEKIPFIQEVASWPVVCATILISSIGIVIPYTTIGKILGFTALPLSYFGFLVVLFLGYFSFGQIIKKGYILVFKTWL

SEQ ID NO: 5 - DNA, 29 bp, artificial sequence
  PH1 Rose/MS fw1
tccnctngatgatgcaattntggcatntg

SEQ ID NO: 6 - DNA, 29 bp, artificial sequence
  PH1 Rose/MS rev 1
canatgccanaattgcatcatcnagngga

SEQ ID NO: 7 - DNA, 28 bp, artificial sequence
  PH1 Rose/MS fw2
ccaagtggaanaagatagatgagattcc

SEQ ID NO: 8 - DNA, 28 bp, artificial sequence
  PH1 Rose/MS rev2
ggaatctcatctatcttnttccacttgg

SEQ ID NO: 9 - DNA, 28 bp, artificial sequence
  PH1 Rose/MS fw3
gnagaagagtatctgttatnttggaaac

SEQ ID NO: 10 - DNA, 28 bp, artificial sequence
  PH1 Rose/MS rev3
gtttccaanataacagatactcttctnc

SEQ ID NO: 11 - DNA, 23 bp, artificial sequence
  PH1 deg.bp4520 F
agtcttgcraaaggagcwcttgc

SEQ ID NO: 12 - DNA, 26 bp, artificial sequence
  PH1 deg.bp3355 F
tcaaagrtgtgcaggtagarttgttc

SEQ ID NO: 13 - DNA, 26 bp, artificial sequence
  PH1 deg.bp6405 F
gcngraaagggagtaaaagcnaaagt

SEQ ID NO: 14 - DNA, 24 bp, artificial sequence
  PH1 deg.bp6650 R
tgcaagtgartcatttaytccatc

SEQ ID NO: 15 - DNA, 24 bp, artificial sequence
  PH1 deg.bp7150 R
agsgtttgcatkagaagyccttca

SEQ ID NO: 16 - DNA, 24 bp, artificial sequence
  PH1 deg.bp4463 F
tggaatctcagttgcawgtgcact

SEQ ID NO: 17 - DNA, 24 bp, artificial sequence
  PH1 deg.bp4463 R
agtgcacwtgcaactgagattcca

SEQ ID NO: 18 - DNA, 23 bp, artificial sequence
  PH1 deg.bp6410 R
actttngcttttactcccttytc

SEQ ID NO: 19 - DNA, 21 bp, artificial sequence
  PH1 deg.bp560 F
tcaaaagcagcmatgaagctt

SEQ ID NO: 20 - DNA, 26 bp, artificial sequence
  PH1 deg.bp580 R
tctgmaagcttcatkgctgcttttga

SEQ ID NO: 21 - DNA, 25 bp, artificial sequence
  PH1 deg.bp640 R
caaytctacctgcacayctttgaac

SEQ ID NO: 22 - DNA, 31 bp, artificial sequence
  PH1 deg.bp1440 F
ggantaarmtagatgagattccttttgattt

SEQ ID NO: 23 - DNA, 26 bp, artificial sequence
  PH1 deg.bp2300 R
cttccyasattggcnatmactgacat

SEQ ID NO: 24 - DNA, 27 bp, artificial sequence
  PH1 Rose bp187(cds) F
tgcaaggaagttggtatcagaacaact

SEQ ID NO: 25 - DNA, 25 bp, artificial sequence
  PH1 Rose bp2030(cds) R
attgtttgcaaggattgcacaactc

SEQ ID NO: 26 - DNA, 27 bp, artificial sequence
  PH1 Rose bp3040 F
ctccgagttgtgcaatccttgcaaaca

SEQ ID NO: 27 - DNA, 25 bp, artificial sequence
  PH1 Rose bp1222 R
ccatgtttcgtattgcagacaagct

SEQ ID NO: 28 - DNA, 23 bp, artificial sequence
  PH1 Rose bp1170 R
gcaagwgctccttttgcaagact

SEQ ID NO: 29 - DNA, 25 bp, artificial sequence
  PH1 Rose bp1460 F
tgatttcataagaagaagggtatct

SEQ ID NO: 30 - DNA, 20 bp, artificial sequence
  PH1 Rose bp2540 F
aaggctgacaatctggagga

SEQ ID NO: 31 - DNA, 23 bp, artificial sequence
  PH1 Rose bp720 R
ccaggaaaaaggtctccaggttc

SEQ ID NO: 32 - DNA, 22 bp, artificial sequence
  PH1 Rose bp740 R
agacaatagtctcacatctcca

SEQ ID NO: 33 - DNA, 24 bp, artificial sequence
  PH1 Rose bp720 F
gttcagactgaattagtagtacaa

SEQ ID NO: 34 - DNA, 26 bp, artificial sequence
  PH1 Rose Stop R
ttcattctgctacctaaagccaggtt

SEQ ID NO: 35 - DNA, 29 bp, artificial sequence
  PH1 Rose ATG Topo F
caccatgagaactttcaaaatccccacca

SEQ ID NO: 36 - DNA, 19 bp, artificial sequence
  PH1 Rose bp240 R
cctctgcctctgtcctcga

SEQ ID NO: 37 - DNA, 22 bp, artificial sequence
  PH1 Rose bp330 F
actgaagctgaaaggagattga

SEQ ID NO: 38 - DNA, 24 bp, artificial sequence
  PH1 Rose bp900 R
cttcaatctcctttcagcttcagt

SEQ ID NO: 39 - DNA, 24 bp, artificial sequence
  PH1 Rose bp1680 R
tccttaaatctagcaatggagtgc

SEQ ID NO: 40 - DNA, 54 bp, artificial sequence
  PH1 Rose ATG+attB1 F
ggggacaagtttgtacaaaaaagcaggctatgagaactttcaaaatccccacca

SEQ ID NO: 41 - DNA, 54 bp, artificial sequence
  PH1 Rose stop+attB2 R
ggggaccactttgtacaagaaagctgggttcattctgctacctaaagccaggtt

SEQ ID NO: 42 - DNA, 26067 bp, artificial sequence
  PH1 Grape cv Pinot Noir
atggcaactcccagatttttcaatggaaattcccttaacataaatgwttwwatacaatatttataaaaaatatattttatttttattttatataattaaaatttttaacaaagaaaaatatcaaaaatataatcaagcttttwtctttttcaattwwwwattttawacattttaactaagaaaaaagttaaaatcacggtcaaattawatwccctwaataaaaagttaaatttgttaattttatattatttttttcttattattattattattattattttatgtttaaaatgttcataaataattaaaataaaaagatgaaatgaaaaaaatgaaattaaaaatcaaaagctaaaatgaagaaataagggggaaaaaaataacgtggcatgttggtttgtcacatgtcaagtttaaaaacattgataagtatgagtaataaaaaaataaaataaaattattataatgtttttgacttgtgatgtcaatttttttttcaaaacaagagaaaagtatggtagttggcggagtgatgaagagttaaaagggtaattttggaatttgttcatgttaatattcaagaacatgtttattaaattactttttaaaagttcaaaaaatctattttttgtttttagtgtacgtttagatagttctcccaaaaaaactttatggactggtacgaggaaaactataaccagcaatttattttatcattatttttatatcaatggatgtattttatttctaatggaatacaatatattaatttaattaatattaaatcaagcaatattaaatttgactcatggaattgaaggtgcataaaataagcccagtggaatctctggctgaataataagcccagtgggcctatcttctgtgtaaaactgtaaacccacccggggctgttgaaatccaggcctgacttagaagaaagctcagtatctagagttgggcctaaagtagccccatcaacaaaaaaatcatggcattgacgtgaatggtcacttcactgacatccatcatgggcagaacaatcttmtgaaggccggttcagtgtgattcctgtcattcaagtaaaacatgtttttccatatgtttcaatattattggtttaatgcagtaaagattgtgaaaaggtcggaaagcccaatcacagagctccaatcgaccgatgggtgtttagctttcttgcatatatgttcggaccttctgaatgcgactgtttcgtcttggtctcaaccatcaaccgggcaagttgtatccaaaacacagtacttgtttmccccaaaccaaaacaacaacagtagcattgtgggccgggcatcacgggtccagacaatgagacggcacatcatattttgttccggcctccgctcctcgttataccgatttatcatccaaaagcaaatttcacttcacttcatggtggacagaaggcacacaaaggaaaagagccagttgtgaagtggtatggggccaaatgcaaaagcggaacaccttccgaaatttcagtatgaagttggacacaaccccagtttggatgaacccatatttcttaatttcaataagattttttttcctccttttaaaaagagggaggtggcatataaaagagggccttgcaagaaaatcccatgaacatttcgattttaacttggttgggagcgagacacatttttgcttgggtcgtcaccctaatttataaagaaaaaaatggatagtgcaagttaaactatgattttgttggacactcctgatataaaggacgacttgatgaaaaaagtaagctgaaaagaataagaactccctcacttttcattctattttattggtctgggttatgctagaaaaatctgtgatggcttaggcgtaaagaggggagagagcacaaatctgcaattggtgtcgttttctggaagacacacatggaaaggaaaaaggcaaaggacatgagtggagaaaaccaggctataaagcattagaccaccctcactcyttttttcttttttggttatacatgattcttgccttaaagtcyycccagaaaattatatcaaaaagaaagaaaagaaagaaaagaaaacacggttaagtgtgtggaacgtgaatsttatcggcgccccaccaattcttattgaaagggagaaagccaaagraaaaaaacaragggttagtaacgtagatcgaccttkgcatatcatagtagatraacagttgtaaattggaattgtatggcgcacaatatcctatgatctaatgattagaagacaccatactagttgtttakgattgtacggtattttaattcaccccattttcctttttctagtctcaaccccaaaagcaaagttgatggaaataaaggacacttaataaattacatgaaaaatagtttttgragcaaaagaaggaatcaaatttgttgtaagatatgactcatattgagtgaaagayatgaaagaaaaatggcaaatgctggagtggagcggtgtgacgatatttatcattcaagtttgtatttttawtattgagggcrgacgataggttaggggtgyggaggggtgttgccyccgatatggttcagaggtatttttggaatttyattacctaakaattaatataaatataaycctaatttgaratatagtwaaagttttattcccacrttaaattcgtgtgttttttttttttttttcatttttctctaattttttcactagaaattgttgcaagaatatcaacaaaattaatgtttattaagcttttcggtgaaatatatgatgatataaataaatggggtgaagatacgagaatattaataaatgaaacttgagtataaastcaggaaactaaagggtgtatgaatgaatgttgtcggaataatgacgtattttgatacttattttgaaaaatcatctttgctcctgaagtagacgcaaatgaagttgggaaattgaagtgctaccataacctagaggctgatggatttttcatcatgccgagcatatgcggggtaccaggacaacccctttcatgttctctatataaaccctaagccatttccaacgacacattaagcctcccaacctttacaaaaggagcaccatgtcatatgatccaacagtgggttctaccaacattgtgaatggtgtcaccaccgtcgactgccaaaagcaagttcgttcatggaggcttctccgctctctyatggagctcctcattccaaggtgcaactgcatttctcttgaagaacaccgaattgaggaagaaaactatctccacagatacttctattcccaacccaccttcatttcctccaccgttgtcaccggcaccattttcgggtaccgccgaggaaaagttagcttttgtacccagacaaactccaagtccaccaacccaattctccttcttgaactggcagttcccacagccattcttgcaagggaaatgcagggtggaattctacgaatcackctcgaatccatagctgccaaaaatggcatggattcttacactctcttgtccataccagtgtggaccatgtgctgtaacgggaggaaagtaggctttgccgttaagcgcacaccctccaaggctgatatgaacgtgctagggctgatgggatccgtcattgtaggtgccggaattataagcgccaaggaactcaactgcgatgatgagctcatgtacctccgagccaattttgagagagttcgcagttcgtccaattctgagtccttccatttgatagaccccgatgggaacatcggtcaggagcttggtattttctttttccgctcaaggtgacctcaatcaagtctaccagaragcaacagcggcaacagttacacccttcgggtttttttgtgttgcgcccgcttctttggataggcaggactttggcttaattttttagcctcccattcatatattcctcttggcctttccagtttgctaattaattaatatgcttgagggagtgtcaacgcatcattatggccgattttggaggggaaggttcatcccataaccttgtttttccttcccttcccttccctttgagtgagcctaatcggccaattggtcatttygctatgtctctgtctgtcttgtggcttattggcatatgtatttggattgatttgaatgaagcatttaaatcctcttcttataatatctaatctagagagagagagacagttactattcataaatggttcttctggtgggtgggtggtgcccgtgactctggcctccataaattagctaacttctatatgggtgacatggaatataatatgtattaatatgtgataaattatcgagtcattggataaggttttaggttaacccagagacggttgtatctaacaaataggttgaccatggctcagcaacttgataaacccaccaaccccgaattaattcagatagttttgacttactgttacgtactggttaggccgtaggcttgtagctaccaaatcctatgacatcctttttttaatgcatgtatattttgtctctttggtgttttgatataaaaaagccaaacaatacaatggattttatggcatgtttttcctttacttcttcactttccaaggaacgaaaagagaaaacaagcaaataaaaaaattatggaaaaatcaatatgagaaaacaaaattagaatacaaaatttgatgaagtatttattttggagtcgaaaaagagaattagaatgaagttgattcctttttaaaccaactatttacttgttgtaaagaagaaaaattgaaatgtataattttaatatagtataataattttacatgaatgcatgataaatgaggaataataatgaaaatttgcaaaagttgttttaatttaaaaatgagaaattattttaaaaattttggaaaaattaaaattcaatgcataaatcatttcaattttaacttccatgytaggacataatcaaaatggtttaaattagaaaaatcaataaagtcrattcgattcaatrttttagtttctacttcatgaatcaaagtacatttattgagtaaaattcaaatttgatttccaactttgattgcaacaagaaattgaaattgttttgattttgcaatctagttaacaatagttaaaattagagtgaaacttttatttcattttcattatggtcttaaaaatcaaaattgacaaagatattgataatgaatgatgattagctagttttcacatagtttcyacttstttgccattgattgaacatttcgaaagattagattttatctctaatccaaaataaattttctcctcataagattatgaaacatacaatacacaaatataatcaatagatcgagattctaatttctactagaatttattatacacatattgaaaagcttcaaacaattatagcatcaacaatgcacaattgatctttaggcttcttaagcatcatcttatataaaaagaaatacgtaaaaaggtaaaaatgataaaaggatatatctcgatctatttactcactttaaaaacaaaaactttatttttattctatttcttatattgcaagtaaatacagaaaaatacttatttttttattttcctttttttcttatctaattatctctcatattattttcctttccgttgcgtcgttaaaagatgggaaacagtaaatgcatttacatcctaaaaatctatttgagaaaaggaaccacacaaaagatatttcaaatatatttgaaggttatattaaaatagaaaataaaaataagaaatcaatttcattctcattcattcatcgaaaaytttgaatttttttttttttttttggaataagaaaaaattattctgaaagcaaaaaacttattctttcattctttcattctatttcacattatgatttacctcgacttcatatatttcccttcctattaacatctaacacaccaagccaagtaaatatgtagtgatagatttagggcatttagtgagtgctgcttgttaaagatataggtggttggggctgcctttttgtgtgggtgtagtgtcgcatgagctggtgttgattccttgtgtcgtttatggacaccagggcgtgtgctacccatttgccttctggatratctatgttatttttaatttcttttcactctttttctaatttgtcatctaattctttttgtttctttgttttcatatttattccgcagactcgtacgtattcctttccaaacaaggtgcgtaaatccttattttggtaaattttattttgggagctattttaagtttgtacggagaaaaattgattaacgactaaataattaagagttcatttgagagtgattttagaaaatatttcaaatatttttaatatttgaatgataaaaattttcaagtattaaaaatattaaattttttttttaaaatcactattaaacaaactttaagaatgcgtttaataaagatttyatgatgcattttttatttttttannnnnnnnnnnntaaatttaagtattaaaaatattagaagtatttcctaaaattattataaaataaattctaaattctttataaaaaaaattattttatctatttaagtataaaattctttaattatattgatgttgtattttttaaatttaaaaatayttttamwaaaaatactttwaagtcaaacattgataaacacattcttaaaacatttttaataacaattctattaataataaattctttaatacttaaawttttttattaaaatrttatttttaaatatgaagaaaaactaaaracacttaacataatcacaaacaaactcacgatagcatgggacttcaaaaggattttgcccgactccagcaattcaccccgcagattatggggttctttggggggttttgtggtacatgaaccggctgagtttcaaatccaaaaactatcttgaacccggttcgggaktagcaatttgagaggtgggaactgggaagcacgatctgcaattcttcacaaactatacctaacggtcwtttgaaaggtggttagtgggaaggaaagacgtggagagtatgggaaagcgagagaaattcaacgagtccatgaaaaaagtaatttattttctatctaaaaccctctcttcctacctctctttcaaaccctttaaccccatgaatgcattctgtggttcatttcctctcttatctcggtgtcatagtagttctcaatcattaccgttgctattatggcaactcccagatttttcaatggaaattcccatcaaaactcctcatcttccaaccccattcgcgaacatcttgtgacgaggcctgatgatcgtaaacatggattcgccaattcggtttcagtttttttgcagcgattcatgtccggaagtaagtctcaaattctccatttttttaaaaacatttttggtttggtttttggagatgtgcttgatgtgggtctttcgtttttcttggaaatgcgtagagaaaatagatggaggatcacggacagaggaagaagagaaggtctactcttggttatatgcattggccaagtcggacaaggacttggtgtttgagtatgttcgatcgactgaaaggggtcagtgtataatctctttttcgtgtgattccattactttgggaatgtaattgttttggcttcagaaatttcgaatatcttacattaatggaagtatgattacgtgtttgatgaaatgtctaggagaagacagacagattaatttttggttgatctggcgtattagcgtataatataaattagtgagacaagaactccatttcaaaattttccccttttccatgatagcgtagaaagttgggggaaagtgattccttcaaaatttaattttctttccttatctttttcttgaacaacaaaagaaaactgtataatttttcctttccttctcttttcctatcaattttctttccgtcaaacgttttttgcaaactaaatataggttatgtttaagttttggaatgtactgaggaaaagggaaaaaaaaaatactaaagaaaatgatttcgtcatgtttggtttaccgtgaaaaatatgaacaacgaaaatcaaatatgattgaaatactttaaacctattttctatgttttaaaaataactttcatctttgtgtaattattttttaaaataactgttagaaaaaaattgaacaaaaaaaatgttatctgaaaatactttatttttgttttaagaatagaaaattgttgtttgttttctggttaccaaacgtgttttttttttttttttttttggagaataaaamtctgtttttgaaaatagttttcaaacaactcctaagattccttcgtggttttcttattcctaatactttctaagagccaaacaaagtattaatgaaatttcatttcctaatttctttcagcatacaataatctaaaacaatttattgagctcaaatctttgaaagaatagtagattgactattacttttgaaaaataaaaataggctaaaattcaataatttaccacacatcctatttctttcacctttcatgataggaaatgtcaagtgttgtatttacctccttaaattaagatgattttttttttctagtgaattgcggacaatcttgccaattaataaaataaaataaaatttgaactaagtttaaatgattataataaagatcttatagattaatgaaagaccttgagtacaaccttgttggtgctttatctattattaaataagtgggctagggtctatcttttagggttaaggagaggaataaatgatgaagtttatggttgcaaaaaataaaataaaaaaatatagaaaagaagagaagaagaaaaagagacaaaaacctagagtctcactaaataaatatttatagaactcttgatttttgttgtgtacatataagacatttatagaaccgataatctaaaattctatatataacatagatgaagcctargcatttaaataaaataatttggatttcttctcaatgaaatgcctaccatttaaaaaaaaagacaggaaataaaagaagaaatacatatgatgtaaatgcaagattcctatttggaatatgattttatctcatatgacatgaaatgggtatcaagtggtgaagggattttattggtcttgtaaaaagtttctccaactacacaagacttgatgagaaaatattagaagataaacatggcaattgataaagaggtcagtgtataaataagctattactaacatgaaatctctttaggactaccccaacttaggtcaagagtgagtgactttcttaacatccatatgcttgtttgttagtggaaaggagattgatttcggttttcaaactcataaaaaataaaaattatgcaaaaaacatgtttggtagcatgttctggaaaaaawttttgttaacagtttatgaaaatgagtcatccttagaaaaacgtagaaatattgttttctttgttaaaaagtwtgaacgctttaacaattggacatgatctaaaagaayaagtagttttttaacatgctcattattttcatttcccaaaatgagaatatttttccaaaaaccatttttattttcatcacttgagaggcactggaccttcctgtttgtaatggaaattgaattgaattttttttttcttcttygttcacatttacctagcatgtcattgtgttttgcaacaatcttacaattcagwgaacaaattgaccctttcagcctcaacctatgtacttgtttcaattatcagattactttactccctttgctttcatgcaggcctgagcttcaccgaagcagagaggagattgaaggaaaatggtccaaatgttcctgttgagtatcgtttcccctcctggtggcatcttctgtggactgctttctttcatccattcaatatcattctgatcgtcttgtcagcactctcatacttagccagtgacaatccaaatggatgcatcatgcttgtactggtttttataagtgtttccctccgattctaccaggtatatgttgctttatttgttctatcaaactggtatcacattttttcatttggcccttaatggttttcctgtgtcttccaggaatayggtagttcaaaagcagccatgaagctttcagaattagtaagatgcccggttaaagttcaaaggtgtgcaggtagagttgttcagactgaattaatagttcaagttgatcaaagagatattgttcctggggacattatcatttttgaacctggtgatctttttcctggtgatgttcggctattgacttcaaaacacctggttgtgaggtatgttacctggaacactwattcaatttatggctcaggattagtgagttctttcatgctatctattccttcctctacagccaattatagaacacgtgacttcctgcttcttgaacagccagtcctcactaacaggggagtctggagtaactgagaaaacagctgacatcaaagaagatcagagcactcctttgctagatttaaagaatatttgttttatggtaggtatwgacattgctagtccttctgtttgatacatatgatatagcttttgtattatattgacaatatttgagtaagcctcatatagaaaagtgaccattaggcaatgctaatggtatctgatgatttgggatcatttgaaaatttattgtgaataagttgggaaaattcacaatgcatgtcaacagaaaraaacacttatagtctttaacaactgacgatcataattctgtgaatttcacaaattattctggagttgtttgtatcatttaggrtatccatgatatattcataagtaattaaaattgggttttttttttttttttttccagtaagacctcccacattgtccatgacaacaaataacatgttggagatattttatggaggaaaatgtccaggttaaaatcatattactgaaaaagctccttccccttcagattttaaaatcatttacaaattattttcatgctttcacagtcttatgtggttaggctttcacctctttccttgaagcatttccaagacaacaacatcagtaattttctttatatgccattatcatgtcaaacacagatatatgattcattttttgtgattccatatttcagggaacgagtgtggtgtcaggttgtggaactggtctaattgtttcaactggatccaagacttacatgagcaccatgttttcaaatatagggaagcaaaagccaccggattactttgagaaaggagttcggcgtatatcttatgtgctgattgctgtcatgctcgtagtagtcactgccatagttttaacttgttattttacatcttatgatttgagtcaaagcattctttttggaatctcagttgcatgtgcacttacacctcagatgcttccactcatagtaaatacaagtcttgcgaaaggagcacttgctatggctagagatagatgtattgtcaaaagcttgactgccataagggatatgggatccatgtaagtttaaattgagctcatgaatgttctggcgcatatattacatcaatatataacaagttacctgccctgaattctctagctaattaacttctgggcaaatggagaagttcccagattttcaccatagattcaagttttaatcatacaattgaggcaaaactttgctttaaatttctcatattcactttttccttttaaccttcaatttttgttaaatcttcaagtaagcagaaactatgcactattcccttttccttcacacatatttaaaaatttttaagctaaacatttattctccttttccccctttttttgttatagggatatcctgtgcattgacaaaactggtacgcttaccatgaaccgtgcaatcatggttaatcatcttgacagttggggtttacccaaagaaaaggtcttgcgctttgctttccttaatgcttacttcaagactgaacagaagtatcctcttgatgatgcaattttggcatatgtatatacaaatggatatcggttccagccgtccaagtggaaaaagatagatgagattccttttgattttacgcggagaagagtatctgttatcttggaaacggagttgaatccaaaagaagattcctaccaatcactcgagaggtttgtggtaaccaaaggagcactagaagaaataataaacctttgttgttttattgatcatattgatcaggatgcaatcacaactttctccctagaagatcagcagaggattctaaatatgggggaggaattaagctatgagggattacgcgttataggagtggcagtaaagaggctacaaagggtatgtgacctatttcaactttcttatttcttatttttgtttttttctcatcttttctgtcttaggttctaattagctctatacattgttgttaaatcatttttcttcaaatagttaatttctttggaatttcattgcttacagaccagaagctgttcattagatgggttgtcaataggctaacatctttcccttcctgcacttactactgaccaagtctagaaatcaccttggtgtcaaccgtaacttgtataatttaaatttaatgtatatttgtagtcaatatcttgaagctagaaggggttaagcacaaaacagtttagtggaaaagttcatagactgacaacctctggctccacgtaatccaaaatctatgtatgggcatgtatataagcatctaaatatatttgatatatttacaattagcacttttgacatatttagctcagtgcttcatcatttgcttgaccttcatttgaatagaaaggttcttatagatcttagtttctgatcaatacacatggattatcttaragtgctgtattaactagagacataggagagtaagacagcttgcaagagttagagcaggagctgcctgagagttaagagcatggactttaaagaaccccacaatgccaaaatatacttgttcaatcatgtatttatgatagtatgttggtttgtttagataagttaaatcatcctcaaatatgagaaggattacagatgcaasaaaggtggattgttcatggttcaataatttgtaatctaaattcttgtcatgaagtttcagattctatatcccaacagccacttcctaaagatttttgaacarcttttggctaaatgtgctagagctcttgcatgttgtcacaggcacaaaattcttacaccagtttttgtgtctgtgcagtgcttagaagctctttaagccagcccacagaaggcatacgaaattacaaggcaccaaccctaacttgtatttttattaccttactgaataagaatacaagacataacttttagcaagactccygcaatctctttagtgagttcatgcttattattaactctctgctaggaggctctccagtcctccctttttatagagtggcgccactccttgttagaytaggagtacacaatcctagttctacttggactcttgttcataggtcatggcatcaaccactcctacttgaactagagatgagcaatcctactcctacccagagcgcaattacaataggaatctgaactcctactcatacttgatttgcaatccygttttgagtccaactcttcatgctgctgttgygtgcctctctctgcagctcgtgtgcatgcaagtccattcttagcctgcatgtctaggtcttccatgtcgaggcaacatgctcttgtatctatcatgttgctgtagtggcaccatgccctggccaagtcatctcttagtgcagctacgagtcatgccattgcacctatgrctcmtcatctctgtgcacaaggcattgcgcctttgtgtcaagccttaggcatcccatctgagtttgcatcactgctgctgcatcaatatgcctcrttcgagtccctcttggtgctgcaacatgccatggcatggcatccatggctcaccaagccgtctccttgcataaggcactgctcctctttgccaccttgtcatgtcatagttcactcatcaggccaaggtgctgccccatgaggccttggtgctgcatggtctgtgtcaaggggctaacatataccaggctcccaagtatatatgaaaaacaacttggtgccaaacctgctaacttgagatgagatagaaaccagtatagaaaattcattaacagttacattacgattttgttygtcttttgcagaaaacaagtgaaggaagcatagatagtgatgaggctaktgaatctgagatgattttccttggccttataaccttctttgacccacccaaggactcagcaaagcaagctctatggcgactggccgagaagggagtaaaagcgaaagtgttaacaggtgactcactgtccctagcagtaaaggtttgtcaggaagttggyatcagaaccacccatgtgattactggacccgatcttgagcttcttgatcaggatttgttccatgagaccgttaaaggggcaacagtactggctcgtctcacccccactcagaaactcagggtagtacagtccttgcagatggttggaaaccatgttgttgggttcctgggtgatggaataaatgactcacttgcattggacgctgccaatgttggtatatcagttgattctggagtctcagttgcaaaagactttgccgatattatattacttgaaaaggacctgaatgtacttgttgctggagttgagcggggtcggctcacctttgcaaacactatgaagtacataaaaatgtcagttattgccaatgtgggaagtgttctttcgatccttattgcaaccctgttccttcgatatgagccattgactcctaggcagctcatcactcagaacttcttgtataattttggccagatcgttattccttgggacaaggtggaagaagattatgtgaagaccccacagagcttttccaggaaaggcttacccatgttcattttgtggaatgcaccagtgtgcaccctctgtgacttagtcacgcttctgtttgtttacttctattatagagcctacactgcaaatgatgctagattcttccattcagcttggttcactgaagggcttctcatgcaaaccctaattatacatttgattcggactgagaaaattcccttcattcaagaggttgcctcctggcctgtgatctgttctactgtcattgtttctgccattggaatcgcaattcccttcacgccaattgggaaagtcatggactttgtccggctgccattttcatattatgggtttttggttgtacttttcattgggtatttttctgttggccaggtggttaagagaatctacattttgatctaccacaaatggctgtaaataacatattgaagtcatgagaagaaagttcgccagagattcaaaaacaggagcaatttttttcctgtgcatattatttagagtaaatgtaacamagcctaaattctctgaatgctttcttagatcattcacaattttcctctatcctttctgctctaacaacattacttgtatcacttgcaaatttgtrgaatatttgagtgtgatggttgaacaacaacaaagaaaggacatggatgagccacatttgtatctccctctttaactctagatcagtacgcaactttggttggatatatcataccatgtgatgcagatagagatagccacacactaatccaagtaacactgcastgttagacacttgcctgtttggtgaacttcctgcatgaaaatataagatggccatggtttattaagtaccattaaacaggaaagaaggtagccaaagaactgtattgacaactctatatgtgtgtgtgcatgtgcattttcaaatcaaatgaaggaaaagatgactgtaggcttttacccaattggagataattgttccgtatgttccattcaaaatcccagtgctttctatcatttttgagtgtccaataagcccatccaaaggaagctgcattataaacctctaactgggttcttccaaaattttgataatccatctgagtagcatttgcracattccattcgttcacccawtycccctgcaaccatgccattttcaagcatcaaaatttgtagtactacttgaattttctaatccacctctatataataagacttttatcaaatgcaaatcagtgttttaagttttggcaattatgactaggttcaaatgttagaactgaagtgtaacaaacttgaacaaaccagtttttgaatggaagaagacagcatggcaattatcttaccaataaaaaccagtggaccatttgctctgttcagagcccgtaattgagtttccctgctgttgtaaataaattggatattgtccaatgggttcatgttaacaaagaaattgtcgaagagattgtagtaatgtaaatctactaccaggttgtaagatcctatgtcagcctgaaaaagttccgayggatctgcaatgccaattctttggcaaactatcacataagcttctgaagaatactttcgaactatttgatagccttgcttgtaatatgaaactaagaggtccagtgaaactgaggcagcagatggttcatttaaaagctcaattcccagcagagtaggatgtttcccatatctgcaaaggtcaaattttagatcaccaccaaccagtacattaactaaggaattggttttgcttcagaagtcrcaagttgcaatgtccaaaggaaaaggattgagattgctcctagatgagtctgtttaaattcattcaacactcatgtcacagaaaataccataagagagagaatgcaagatgcagaaaatgaagagaatacttaaaaaacagttaaaagaaacatgctacacacacacacacacacacacacatatataatattaatgcctttgatggaaaagctacaattatagatgctagtagaagagggagctggagcaatgctgcaatggcaatgaagcagggtaactctaggagcagcataaacttcacttaatcttgtaggtctagcatgtgaatgaggatgtgtgtttgggatctgagaaaaaacaactgaacagttttttggagcaatttgatggagattyagtttggtgattgtttttattttttattttttatttctgtaaaacacttattgaaatcagccattttttgcatttcaatgatggaagatcattgatctaatagtgatatgttyaaatgccatggaatcagwagcatcatttckatttcatatggtatgcaaacaaaccataataacaatattaggctccccagctgcaagccaacaataccatcctaatcctaatccatgttggccaaggcaagtatgtaactgccctttagaagcaagaggatkccccatgttaatccaacactggcaggacacatgcaaattggcaaatttgtaacgagtaactggtatcttaaccctccaataactggtctagaatgtacctggaagctaaaaattctatcacatccaatgtttgtgaaatgtaacttgcagatgtaggccagccagatgaaccatctctactagcactatgttccatcccattctgggagccaggagccgcatgcaggtcaattatgcaccttatattataggctctgtgcaacagtttttcagatacatatcaaaaggatgcaaatttaaagtcccaacagtgaaaacagaaacaggttcaaaacaagacctactgcgcccatgagaatgcattatccagagcttccaaagttcccccaataaaaggagccggtggattaggatcaaaagcaatccaccaaccaacaggaatcctcacagtatttattccatgtctatacagaaaaatgaaatcttctatagtgataaagctgtttctatgtctctgcagttttcaaaaggccaatacatatcaaataaacaagatactgagattcatggagaaacctgttctacaatctagcaaaaatagctcaayttctgaaaatcacaaagtgatatccaaaatattatrctaactggctaagatatatggttcatgcttrgcatatagttcaatcaacaaatcatgatgacaatattcacaagtgggttaaaataagaacattggaggatgccaaaactatatcacaaagtaaagctcatataccaactatgtgtggacacaggtttggggatggacacaatactracaagccaggagttttttgttgcttaaagtgcccccaaagaaacctttctgcagaaaattaaaaatgttttgcaaacatacaagtaaaattagttggggaaaaatactttgttgagtttgagttgtagatacaatattacatttaagcatcacctcttggcaattgtgttttttttataggtaaatttttgttcctattgggacttgaacctggaacctccaacaaacctcccatcctttactgcttgagctaggcctcaagggcatcgcctcttagcaattgttgatactcaatagagttagatatcaaaacattacatcatattaaattatacagcataaaacaaaggcaaaacatgctctgggctccagattccttaacactatattgggttgtttttttcttagccaaacctgttatatcctaaagtccataacccataaattatgcaatatgagttcatccaataaattctgttaaaggtgctatccaatataattggtcaagaagtttgagaaaaaaaaaagaaaaaataaataaaagaagtcaaacaggaaatttggagctaccttaagaacttctttggctttatcatgtccatacccatttgcaagctggtaatcaccatgtatgttatttgctacaattgtcatttcaaatgtggctgcattatcatcccatcctggcatccctggatagtctgctgagagctgattagctagtgtagcctagacaagtgaagtataagactatcatgttttagtattagaaaataaaagaggggaccttgaagagatattaagaaaatgtcatctaggaggatacttggtaagtttggtaatggaatagtgggtgggtgactgggtattccaacaagcatttataccagtatttgatgacagcaagctaaacaaaaactcaaacaatctgagattgtaaccaacttaattacttgcataacattgaacaactggattaagttttttaattttccctcaactattcctaaactattcctgaaattctagaataatcctgaaattattactgactactcaacttccagcactattcttaaataattcacaaaggcagataggtgccaaaatctgctggtcttgctgtgggcaatcagagmtattgacaatggaaccatagagaagccaaaacaatggatgactgaaaatgttcaacctggagctctargaatggtagtcatgccacactgcaygcatgacaagtttgccattacaccattmttgactgtctacccaactaaagtgagtaaattaaattctgaaatagtaaagtaagcgattgaatacacctgcagatagttcccattcttcagtttgatgtgaactctgttgtcataatttctttcaacataaaatgtttcctttattgacgatgatccygccattgcagagacagagcctccctcaccatcgcatgytaaaaactgcccttgagaagtgcggaactgaaactctgaatcagaaaccctccataactgcaggacactaatgaaataattaatttccatacatcacaagaatttgtaatatcaaacagcctacctttgattcattagattatgacaccctgtttggtgcacctaatgagataactgtatttgtttaatagatacaagtctaaaagttaatccttagaataaatgcagttattatcatcatcctgcatgtgcttcaggcaaagacaacaaggtctgaacttaagaactagattaataacaatgattccaaccaagacaccactaacacattgaataaccagcacaatgcacaattaactgagaacctagaaattctacgattcaatttctaaataagcattctacactaatttctggaaataacagtctcttgatttaatatccaactccccctccaggaagcaaggaaaatggaagggaaaaaagaagggaaaaagaacacaagctgatgtttgaaaatatatcataagcaattttgtttcacaagaaaacaagtttatttcatcctttttataagcagcaccttcataaaatcaccaataactaaatttttttctatttaagtcatgatgagcaaacactggagtatttcatataacttaagagtagaatggtgaatttatccaaattctaagatccgaataactggtacaaggcattgctaagtggttttgagttgagaaattgttgtatgtcccattaacaaagcgcatcatccaacttaaggttgttgaccatgtgaaaaatgaagtcaaaagcacttcaatgctaatttgccttagtttatgatagtgtgtattatctagcagaggaacaagtctcagacagatacagccttaggacagaaatttctcatggtagttttcttcctttccttttcattwactagatctaaacacagcaatttcaggtttgcttagcattggtaagaatgctgccactagatctaaactgaacacaataccacatgtttccttgcattattaaatgatcactcacatgcctcacatgactcaaccacctaatgaactttgaatacagaaatcagaaataatatattcaacatcctcatgaataatgcttgagaagagtgcagcacactctttggatcatttcgatgtgcaaactgttgctcgatcttttgacatcacttcaatttttttgctttgaaagaaggccataaaattctaaatatggaccacaacagyagaactcraattttgctaacctcaaccaagttgtgcttgtgtttmttacctcattgctttacttttcgagtcaaactcaaccatgtaataaaaggagtttaaragtgttttcccagggtagaggctagaattcaagcataatcttttaataccacaagttaaaagttctaatttaccacatatcttyaatttctaaaagtgcctatcaatatgtatattcaatttctagaagtagttgcatagaccttctcttcccccaacaatatatatatatatatatatatatatatatatatatatatatatatattgctgctggaagcttawaatttcaatttcatgttgcagtaacaggctggcaaagaaatgtgtgaagttgaactaattctgttgtaagaatgcatgttaaagtgttcaatcaactccattagcagttaattcatgttggaatggatgaatttctttacctccttaaaattgcagctacacacctacaccttgaattaaaatgactggccaaagagttaaaactacgagaactagtcaaaaagattatcataaattttttggtgaacaggaaaaaaaaatttaaatgaaaatatctgattatatgatcaaatctactaatttaaagaaaccacttatttatgacataaagtatcttacacagtaacatataaaaatcaagagaaaaaaattaagacaagcgcataacaataggtcagacatctaaccctgaaggtttcccatgaagaaggaacatctttgtctactgtaacacccatgcctcctccattctctgcagatacatacttctgcaacatcagtgacttgaattgaacctctgttccatcctaaatttccatagccagaaaagggttagccagatcagtmacctaaatcataaaagaatgccaagccaatgaagcagcacaattacgataaaggaaagcttacgagcatgtctccatttggaatgccatcaaacaatgaaggtttaatccagccttccacaaccagccaccctcccaggttcactcctctaactttttcacccccttgtactaagtccacttgaatcaaacaatagaaaatgttttataatttgcaaattgggttctgttttccctttttttttttcatcctccaaatacacagcatttcaagcaacaatctaaaatcaagaaaaaggcgacaagcaaratagaaatgcaggtgcccattcygataaaaaaayaatgggttcttgttcttgtcgttcccayactcgaatacattgcatttccaagaaccaaacgaaaaaaaaaaaatcaagaaaagggagttatagaatttacccgagtatgagaaaataagccgacaacagaggagaaatgcaaatacccatttgcggaaaacgagttccatgtagaatgcaggcggcggaaacatcaagaaggcgcaaatatagagtccatctacaacaaaagaacaacycaaataaagaaactgagattgtttgaaaacccacttgggcgaatcttagtaattctttccttatcactcaaattattccaaacataaacaattaaacaagcatmaatatttagagytagcctctgcgattattrcatcattaattaaattgagaaacccaagtgtagastcgagacaagctaaaacccattaaaataagagcagaaggaaagaacaatcttatttttattccaacaatgctttaaaaaaagggtggaaagttttcacgtgtacggcatagaatgtggattgaaaagggaaggaaagggcgactttgagtgaagatttgggatgctgggtcttgattggttggatgaagaaaaggttagaaaggttagagaacgtgcctttaagtaaagaaattgggaaagtattcgagatgacaatggttacagtgcaactccattacaccttatcttcacgttcgatcttccggctcccaccaccttcctttgctaaatctgggttcagaggaaacggtctacgactttctttttctttttctaatttcttcttccttctattatttcctctcttttcttttctcttcttttcttctattttcccttcatacatttaagaaaagtataggttgaagatcactgataatcaaattatttaatcaacatgttttgatcttcagctaactcatgaagatcttccttcgtctttgagagaaaaataatatacccattaaatcaaagggygtagcatcwttatatttgmaccagtaaagccaaaagtcttasaagaataccagctgctcgttggagacaaagcaasttagaggtacatttcaacagtctgataaattcattttgagaatttttrcaggccgctcagtatgggccttaaatgtttcctaaaccaaaagcctarcccaaaatgaagacctttcccaacgatataagagacagagacagagccagaggatttattgtgagacaagtacaacaatatagcctgggccctaggcttgaccttgagcttgagcccactttacttctaacaggctctcaaagcacaggatcaagcgtgaggcactttgagagaatgccyactcatcttctttccttgggtgacttcgcactttgcatacgttttgggggctctttgtgcacactcaactcttttctatacctataaacatgacttttattccaataaaattgtactagcacaaaaaattattgtataaggacaaktttcacaagtttggcttactattggactttcaaggccaaataagttaacaagagacttttattgaattaagtgagaaattataccgggaatattatataatttgacaaaacccctttaacaagggaagtcccccaaaagcaatgtagtaaatattattttatagtactctcatttttgtctttcaattatggaacttcacaatagctattagagtgtttgacactgaggctgatatattagtaactctccgttgatgttgatgtatgcagtgcatggtgccacgctagtggttttacttttacatacctaatatgatatgacccatattagattggcatcctacatgattacttgagcaaggatcctatattgaggatcaaaatattaattaaacttttttcccaccgttgaattaggattagatactacttacttcttcgattcgttgatttaaactcaacaaatgggaatttgttataaagtattactccattgatgccaccattattgatatggtaatacctaccccaattaaagcgatagaaagctattgtgattgacattgaaaatataattgtatttttatgatgagatgtgtatgactttttttattttttttatatccaattcttctgacatcaactatttgatttgatggcgtacatcatatcttctaattctactcttatatcttgttctcctttgatatgacatttttattttctataaaagcaccattctcatcatacaatttataattatcatcatatggtctaaaatttttcatttgttactttcctctctagtccacatgtgaccaaccaaaatattgcctcctaacataccattttatgaatttaacagagatataataaatatatatattaaaatttttaaaatttttattaacttgatttatgataaagttgtttgataaagaaaagaagtatatttattagttatgatgtaatttttacattgaaaatatctttgaaaggaaaaactttatatgaaaattaatttaaaatttatattttctcatggcctaagggttataactttataccataaaataaatagtaacttttcctccacaaaattttatttattttataattttataaaataatttcaaaaattatataattgttattattattattattttttattcaagtggcctttgyatgtggtggtgccctattggtttaaagaggtgggtatgatatccaaaagygattttcggtacccgtagtatcattttttgatatcctagcataattcgatatgagaattaatwactctcatttaaaaaaagaaaggaaaaaatacttatcaacaaagatgtaatttttacattgaaaatgtctttgaaaggaaaaactttaatataaaaattcagttttcaaataattacttaaaattataaatarataawtttaatataaatttaagatccaacaaaaataattaaaatataatcttttgaatattgaactttaatataaatgatacttcaatattaatttatatattacaaagttwctatttaaaaaacaacttcaatataaattaattcactaaaacttgaaattaaatagaagcaggtgtcatatgaggaaataaatttacttaaaaatcaaaagtgaaattaatttttaaatattttttttttcaaaatatacaaacggcttaaatttgtcaaatccaattgacaattttccaagtttggcttagtattggactttcaagggcaaataagttaacaagagacttttattgaatcaagtaagagaaattataccgggaatattatgtaacattgattagactatactgattgacaaaatccctttaacacgggaagtcccccatatgcactgcatggtgccacgatagtggttttatgcacctaacatgatatgacccatattagaggaagcctccctacatgactacttgagcaaggatcctatattgaggatcaaaatattaattaaatttttttcccaccgagggataggaatttgttataaagtattactccattgatgccaccattattgacatggtactacctaccccaattaataaaaagctattgtgattgacaatgaaaatataattttatgtttttgatgagatgtctatgtcttttttttatatattttttttatttttatatctaacttttttgacattaaccatcttatcattaatttgacttaatttgatggacgtatatcatatcttctaattctactcttatatcttattctccttcgaccatgacatttttattttctataaaagacatttaattctcatcatacaacttataatcaccgtcatatgatctaaattttttctat

SEQ ID NO: 43 - protein, 942 aa, artificial sequence
  PH1 Grape cv Pinot Noir
MATPRFFNGNSHQNSSSSNPIREHLVTRPDDRKHGFANSVSVFLQRFMSGKKIDGGSRTEEEEKVYSWLYALAKSDKDLVFEYVRSTERGLSFTEAERRLKENGPNVPVEYHFPSWWHLLWTAFFHPFNIILIVLSALSYLASDNPNGCIMLVLVFISVSLRFYQEYGSSKAAMKLSELVRCPVKVQRCAGRVVQTELIVQVDQRDIVPGDIIIFEPGDLFPGDVRLLTSKHLVVSQSSLTGESGVTEKTADIKEDQSTPLLDLKNICFMGTSVVSGCGTGLIVSTGSKTYMSTMFSYIGKQKPPDYFEKGVRRISYVLIAVMLVVVTAIVLTCYFTSYDLSQSILFGISVACALTPQMLPLIVNTSLAKGALAMARDRCIVKSLTAIRDMGSMDILCIDKTGTLTMNRAIMVNHLDSWGLPKEKVLRFAFLNAYFKTEQKYPLDDAILAYVYTNGYRFQPSKWKKIDEIPFDFTRRRVSVILETELNPKEDSYQSLERFVVTKGALEEIINLCCFIDHIDQDAITTFSLEDQQRILNMGEELSYEGLRVIGVAVKRLQRKTSEGSIDSDEAIESEMIFLGLITFFDPPKDSAKQALWRLAEKGVKAKVLTGDSLSLAVKVCQEVGIRTTHVITGPDLELLDQDLFHETVKGATVLARLTPTQKLRVVQSLQMVGNHVVGFLGDGINDSLALDAANVGISVDSGVSVAKDFADIILLEKDLNVLVAGVERGRLTFANTMKYIKMSVIANVGSVLSILIATLFLRYEPLTPRQLITQNFLYNFGQIVIPWDKVEEDYVKTPQSFSRKGLPMFILWNAPVCTLCDLVTLLFVYFYYRAYTANDARFFHSAWFTEGLLMQTLIIHLIRTEKIPFIQEVASWPVICSTVIVSAIGIAIPFTPIGKVMDFVRLPFSYYGFLVVLFIGYFSVGQVVKRIYILIYHKWL

SEQ ID NO: 44 - DNA, 2829 bp, artificial sequence
  PH1 Grape cv Nebbiolo
atggcaactcccagatttttcaatggaaattcccatcaaaactcctcatcttccaaccccattcgcgaacatcttgtgacgaggcctgatgatcgtaaacatggattcgccaattcggtttcagtttttttgcagcgattcatgtccggaaagaaaatagatggaggatcacggacagaggaagaagagaaggtctactcttggttatatgcattggccaagtcggacaaggacttggtgtttgagtatgttcgatccactgaaaggggcctgagcttcaccgaagcagagaggagattgaaggaaaatggtccaaatgttcctgttgagtatcatttcccctcctggtggcatcttctgtggactgctttctttcatccattcaatatcattctgatcgtcttgtcagcactctcatacttagccagtgacaatccaaatggatgcatcatgctcgtactggtttttataagtgtttccctccgattctaccaggaatacggtagttcaaaagcagccatgaagctttcagaattagtaagatgcccggttaaagttcaaaggtgtgcaggtagagttgttcagactgaattaatagttcaagttgatcaaagagatattgttcctggggacattatcatttttgaacctggtgatctttttcctggtgatgttcggctattgacttcaaaacacctggttgtgagccagtcctcactaacaggggagtctggagtaactgagaaaacagctgacatcaaagaagatcagagcactcctttgctagatttaaagaatatttgttttatgggaacgagtgtggtgtcaggttgtggaactggtctaattgtttcaactggatccaagacttacatgagcaccatgttttcaaatatagggaagcaaaagccaccggattactttgagaaaggagttcggcgtatatcttatgtgctgattgctgtcatgctcgtagtagtcactgccatagttttaacttgttattttacatcttatgatttgagtcaaagcattctttttggaatctcagttgcatgtgcacttacacctcagatgcttccactcatagtaaatacaagtcttgcgaaaggagcacttgctatggctagagatagatgtattgtcaaaagcttgactgccataagggatatgggatccatggatatcctgtgcattgacaaaactggtacgcttaccatgaaccgtgcaatcatggttaatcatcttgacagttggggtttacccaaagaaaaggtcttgcgctttgctttccttaatgcttacttcaagactgaacagaagtatcctcttgatgatgcaattttggcatatgtatatacaaatggatatcggttccagccgtccaagtggaaaaagatagatgagattccttttgattttacgcggagaagagtatctgttatcttggaaacggagttgaatccaaaagaagattcctaccaatcactcgagaggtttgtggtaaccaaaggagcgctagaagaaataataaacctttgttgttttattgatcatattgatcaggatgcaatcacaactttctccctagaagatcagcagaggattctaaatatgggggaggaattaagctatgagggattacgcgttataggagtggcagtaaagaggctacaaaggaaaacaagtgaaggaagcatagatagtgatgaggctattgaatctgagatgattttccttggccttataaccttctttgacccacccaaggactcagcaaagcaagctctatggcgactggccgagaagggagtaaaagcgaaagtgttaacaggtgactcactgtccctagcagtaaaggtttgtcaggaagttggtatcagaaccacccatgtgattactggacccgatcttgagcttcttgatcaggatttgttccatgagaccgttaaaggggcaacagtactggctcgtctcacccccactcagaaactcagggtagtacagtccttgcagatggttggaaaccatgttgttgggttcctgggtgatggaataaatgactcacttgcattggacgctgccaatgttggtatatcagttgattctggagtctcagttgcaaaagactttgccgatattatattacttgaaaaggacctgaatgtacttgttgctggagttgagcggggtcggctcacctttgcaaacactatgaagtacataaaaatgtcagttattgccaatgtgggaagtgttctttcgatccttattgcaaccctgttccttcgatatgagccattgactcctaggcagctcatcactcagaacttcttgtataattttggccagatcgttattccttgggacaaggtggaagaagattatgtgaagaccccacagagcttttccaggaaaggcttacccatgttcattttgtggaatgcaccagtgtgcaccctctgtgacttagtcacgcttctgtttgtttacttctattatagagcctacactgcaaatgatgctagattcttccattcagcttggttcactgaagggcttctcatgcaaaccctaattatacatttgattcggactgagaaaattcccttcattcaagaggttgcctcctggcctgtgatctgttctactgtcattgtttctgccattggaatcgcaattcccttcacgccaattgggaaagtcatggactttgtccggctgccattttcatattatgggtttttggttgtacttttcattgggtatttttctgttggccaggtggttaagagaatctacattttgatctaccacaaatggctgtaa

SEQ ID NO: 45 - protein, 942 aa, artificial sequence
  Synthetic Construct
MATPRFFNGNSHQNSSSSNPIREHLVTRPDDRKHGFANSVSVFLQRFMSGKKIDGGSRTEEEEKVYSWLYALAKSDKDLVFEYVRSTERGLSFTEAERRLKENGPNVPVEYHFPSWWHLLWTAFFHPFNIILIVLSALSYLASDNPNGCIMLVLVFISVSLRFYQEYGSSKAAMKLSELVRCPVKVQRCAGRVVQTELIVQVDQRDIVPGDIIIFEPGDLFPGDVRLLTSKHLVVSQSSLTGESGVTEKTADIKEDQSTPLLDLKNICFMGTSVVSGCGTGLIVSTGSKTYMSTMFSNIGKQKPPDYFEKGVRRISYVLIAVMLVVVTAIVLTCYFTSYDLSQSILFGISVACALTPQMLPLIVNTSLAKGALAMARDRCIVKSLTAIRDMGSMDILCIDKTGTLTMNRAIMVNHLDSWGLPKEKVLRFAFLNAYFKTEQKYPLDDAILAYVYTNGYRFQPSKWKKIDEIPFDFTRRRVSVILETELNPKEDSYQSLERFVVTKGALEEIINLCCFIDHIDQDAITTFSLEDQQRILNMGEELSYEGLRVIGVAVKRLQRKTSEGSIDSDEAIESEMIFLGLITFFDPPKDSAKQALWRLAEKGVKAKVLTGDSLSLAVKVCQEVGIRTTHVITGPDLELLDQDLFHETVKGATVLARLTPTQKLRVVQSLQMVGNHVVGFLGDGINDSLALDAANVGISVDSGVSVAKDFADIILLEKDLNVLVAGVERGRLTFANTMKYIKMSVIANVGSVLSILIATLFLRYEPLTPRQLITQNFLYNFGQIVIPWDKVEEDYVKTPQSFSRKGLPMFILWNAPVCTLCDLVTLLFVYFYYRAYTANDARFFHSAWFTEGLLMQTLIIHLIRTEKIPFIQEVASWPVICSTVIVSAIGIAIPFTPIGKVMDFVRLPFSYYGFLVVLFIGYFSVGQVVKRIYILIYHKWL

SEQ ID NO: 46 - DNA, 39 bp, artificial sequence
  PH5 Phusion PCR
cctattcatcgtcgacacatggccgaagatctggagaga

SEQ ID NO: 47 - DNA, 34 bp, artificial sequence
  PH5 Phusion PCR
cgggatcctggagccagaagtttgttataggagg

SEQ ID NO: 48 - DNA, 50 bp, artificial sequence
  PH1 Grape cv Nebbiolo
ggggacaagtttgtacaaaaaagcaggctttatggcaactcccagatttt

SEQ ID NO: 49 - DNA, 24 bp, artificial sequence
  PH1 Grape cv Nebbiolo
tctagcaaaggagtgctctgatct

SEQ ID NO: 50 - DNA, 22 bp, artificial sequence
  PH1 Grape cv Nebbiolo
cactaacaggggagtctggagt

SEQ ID NO: 51 - DNA, 26 bp, artificial sequence
  PH1 Grrape cv Nebbiolo
atcttctagggagaaagttgtgattg

SEQ ID NO: 52 - DNA, 23 bp, artificial sequence
  PH1 Grape cv Nebbiolo
tcactcgagaggtttgtggtaac

SEQ ID NO: 53 - DNA, 50 bp, artificial sequence
  PH1 Grape cv Nebbiolo
ggggaccactttgtacaagaaagctgggtattacagccatttgtggtaga

SEQ ID NO: 54 - DNA, 54 bp, artificial sequence; identical to SEQ ID NO: 40
  RosePH1

SEQ ID NO: 55 - DNA, 54 bp, artificial sequence; identical to SEQ ID NO: 41
  RosePH1

SEQ ID NO: 56 - DNA, 30 bp, artificial sequence
  PH1 Phusion polymerase
caccatgtggttatccaatattttccctgt

SEQ ID NO: 57 - DNA, 23 bp, artificial sequence
  PH1 Phusion polymerase
taggactaaagccatgtcttgaa

SEQ ID NO: 58 - DNA, 7383 bp, artificial sequence
  Petunia PH1 genomic
atgtggttatccaatattttcccagtaaatcactctaacataccttattataatatttctcaaaatcttgttcaaaaacccagtggacaaacgcaacataatgatggtcctaacacttcagtgttctttcgtttcttgcggaggttcacttctgcaagtaggcttcctttaatttctctacctatcttttgtttttcagtttttgcatgcgcgcaagttcttttcctagaatgtattgaattatttgaatatctatgttttgaaacattgtagagagggtacttttacttgtttatttttagtttatcacacgttactcatatacgtacaatccagtactaatgaatgtgtcaactcataactgcatgctttcttactccattatttttcgtcaaattaaagttgaatccatttaatttgaactagctaacccagtgtgatctcgcaaataaagtctagggagggtgaggagtacgcaatccttacccctaacctttgaactataactattgaaagaacatatatgcagcggaagcagttcaaacatcatactcacatgtattctagacaaactagtagcaagcattcagttttgcaatgatataaaacggaatacctgttaaagcccacacaaagaccttcaataaccttgttgtccagttgctccagctttccacggtcttctgttgtgtacccgatcaatttccggaccaacagtacttgggtggaacaagtgcttcagagaaagagggtgaaaaatatgtatttttcgtgtgtctacagtggaggccgaaatcctccttttataggttaagttttgagggttaaaacccttctccaaaacctgttaggatttcccttttccaccaatcaactttctcccagtccaaattggattcggtcacaacagggaccacagaatcaattaataaggctttcaattatggaattaattctttttctctagaattaacctttccataataaattacaaattatttccactagaaattcgtaattgcactccttagataaatttcgaattcttccatcaaatcttatttaactccccacgttaagattacagacaccaatcaataatattaaattactgacaacttaatatattgattaaatgaatatcctttagatttccgcttaacttattccatgtgccggatacaaaattcactagctaggtttacacatagaagcttataagctttcataaagggatgtcatcaatctctatagcgagacgtggattctatcaactagctattacttcgcaaatgcatattatcattatccaacttatcaggaattatttgacccaaatatcctggacctcacctattgataaatcaagacaatgaataatatacatcactcataatagctatatcaagattaagagtataagtacatctgatgagctagagagtttgttttatatagtcagtataaaaacacttatctctacttggtccgttcaatacacacaaagtgcactagcacaagaagttggaactataccgttcccataatcaagataaattatatataatcttgtactacaatcataccgatggttttgtccaatttccatcttagatcgcgaacactacttcattatctataagaaccgatgatttaatcttccgtgtataagcttggctctacacactaaatcaactactgtataaataataggacacacatgacacaattgatctaaataaataatactttattatatttaataaatgtataaagcaacaattgtttaataagaaataatctaactaacaactgcatggttattagtatattttccaacaaactatatatgtaggttttttctttcaactacctcattgtgcgtttacagcttttactattttgcttttaaaattcaaaatgttctatactcgctactagtatttcgttctaattgtctagttctcttagctatcgttacatttcttaactcatggaaaatggaaagacagaagtataagcattagtgtgagcctgattaaagaactcttcttacttattctgataccatgttgaactgcggactcatttaaactttacactatcgaatagggaggattgtgtttgtttattttaggtatgtattttgggacaatatgtactttacttgtgtttgagttttataagtgaaatggatacagggtcgttaatcttttgttctgttctttatgtatatccatataaagagaaaattgatggagggtcgagaactgaagaagaagagaagttgtattcttggatatatgctttggctcaatcagaaaaggacttggtgtacgagtatgttcaatccactgaaagaggtaattttgtcaacataccaatttattttttatattttctattttcttgttgaagcaatggtagctatagaaaaacatgaatgtgttgtttatttcttggctaagtctagtttcaaattgatgttctaatgcgccaatgcgtaatttatctctgatcgtttaaagttaacaaatgaattgaaagttcatgagaattttgatttaggaacatatgcatatctttcctggataatactataaggcttatgttaagtaagaagctgtgttgaagaattgacatttgaaaaaacctgcattgcactagtcgtcactgacaaggatattcacaaaatgtatctattgtgttcttataaactagtctttcagttttgttcactttatactattgaacatttccgtctttttttgattttctttctttaagttaattttatttgatttataggagaaacataaatgtcttaagggaatgtataaccacacttttcatttctttgaatagggctggcaaagttgtcatatttcaattttggtatgtcattaggttgttgttatgcaggcttgagctttgctgaagctgacagaagacttaaagaaacaggaccaaatattcctcttgagaatactttcccacagtggtggaatctactgtggagtgcttcattccatcctttcaacataattcttcttgtcctatcagtactctcttacattgcaagtgacaatccaaatggttgtatcatgcttatattagtcttcataagtgtctctctccgcttttaccaggtatcgtccagttcacagtttcgatacaaatacatgtgcacatatatatacacgttgattaagatctaatctggagtttttttttctcctcaggaattcagcagctcaaaagcagcaatgaagcttgcagagtttgtacggtgtcctataaaggttcaaagatgtgcaggtagaattgttcaaactgaggtacaggttaaagttgatcaacgagaagttgttccaggtgatatcgtaattgttggaccgggggatcttttcccaggtgatgtgaggctactagaatcaaagcacctagttgtaaggtaaaattatagtaatgttatccttgtagactggagacgaaaattatatcatatctcatgatttgttctcatttgttttgttaaatgagttcatcgcatatttgtgaacagtcaatcttcactaacaggcgaatctgcaacgactgagaaaacagcttacgtaagagaagataacagcactccgttgctagatttgaagaacatttgctttatggtaagtccgggcattatagagggctgttctgtttcttgttctgacttttttgctgtgacctaaaaaagggtaaaattttctacattctgagaatccccatttcaatttaaatatatactaaatagaagaaaagtatgtgttattaacagaatagggagcagtactttatttattacattcagtggttgaagaaagaaaacaatgaatacaaacctttattcagtcattgtaccttcaaaatttctttatatcatgaggattcacgaatgcacttgcacttaaggatcatgcgaagtatgcatgcataagacattcgtttcactacaaagttgcaggtaaatatctgtatttaccaatacaatgtcttcttgttattagaacgcaaccagtaatcctcgctaacataatatcaatagtcacagttagtatgcatcattttgtattgtagcaaagtttggtgatccttttcatctttaagttctatattttgtatttcattgagagagtcgtgcttcatttcagggaacaagtgttgtatctggtagtggaaccggtctggttgtctctactggattaaagacgtacctcagcacaatcttttcaaaagtagggaagaaaagaccagcagatgattttgaaaaaggcatccgccacatatcatttgtgcttatcagcatcatgcttgttgtggtctcagtaattgtcctatctgtttactttacatcacgtgatctgagtaagaccatactgtatggaatctcagttgcaagtgcactcacccctcagatgcttcccctcattgtgaatactagtcttgcaaaaggagctcttgccatggccaaggatagatgtatagttaagagtttaactgctatacgaaatatgggatccatgtaagttgtatagttcatgtccatagtgacttttttcacctctagatattttctatctctaatccttttttcccctattttcaatttgagaatgattgatccagcttgattctttgtcttattcttttcatttgtttccattttcttcttttatttccagggatatcatatgcatagataagactggtacactcactgtggattttgcgactatggttaattacttcgatagctgggggtcaccaaatgaaacagtcctacactttgccttcttgaatgcttacttccaaagccaaaataagcatcctctggatgatgcaattatggcatatgcatacacaaatggtttcaggtttcagccttccaagtggaataagatagatgagattccttttgattttacaagaagaagagtatctgttatattggaaaccaaaattagcgccaaagacgagaaaataagtggtaacagagtgttgataacaaaaggagcactagaagatattttgagaatatgttctttcgttgagcacatagataagggtgtgattttaacttttaccaaagaagactacagaagaattagtgacctggcagaaagattaagtaatgaaggatatcgggttcttgggttagcaatgaaacaactcctaccagtaagtctgaattcctagacacaattttcataatactagttagcttctctcatagtgcaaattcctcggacaaatccatttgaatctgcaatattctgcaagttctcgatgatcataactcagatggtagttctgaactcagttccaagtttattactgttgaagagtgtcaccatcccatctaaaagcttaagcgattagatgtggtacactttatttatttaattgctttctcaacatgccccctcacgggcgggcttgattctttttcataagccaagcacatggaaattctttttttttttaaataatggatggcagtgaggttcgatcccaggacctctgcctggttgattttagatgagatggtcgcacacttcaacaattacatgtcatatcacagttgcattagtatgttcttgcagtatgaaactcaaatcactatagatagttaagatacaggtcaattataaccattaatttttatgtaacaagccggttagctaaatgattagatataatgttaactagataaacttcaattatcttcatagtgtaacataagtttgtgtatgtatttgagatatcaccaactttggtgaaattgatgtccacttcagtaatgtgctgtccaaattattcattttgttgctgccgagcatgaacacaaatttgtttatagaccaaaaaatttggaaaatacttaccaaggtttctgaaactttttttgatatgtcctagtcagctttcagtgtaacagtcataagctcttctcaagttggagctttgctagttcttaaatccttttaacagttggatatcagataaatagacaataactttatccccttttgcactcagaatgccgccttttactttcagtttctatgtttctatactaataagaactcgatcggaaacactactataagcattagacctttgaggcctgccaaggttcttaatgcaaggataaatgtgcactttatatgttgcttcataattattactgctgtcttgcaggaagtcaaagttagcagcatgatctatgaggaggacgttgaatccagtatggtattcgttgggcttatatccttttttgatccaccaaaagactctgcaaagcaagcactatggcgcctagcagaaaagggagtaaaagctaaagtactgacaggtgatactctatctcttgcgataagaatatgcaaggaggtcggtataagaacaactcatgtcatcactggacctgaccttgagtcactagacacagattctttccatgagacagttaagaggtcaacagtttttgcccgacttacacctactcagaaactaagagtggtgcaatctttgcaaacaaagggtgatcatgttgttggtttcttaggagatggagtaaatgattcacttgcactggatgcagcaaatgtaggtatatctgttgactccggtgcctcaatggccaaagactttgctaacattatcttacttgagaaagacctcaatgttctcatagctggagttgagcaaggccggcttacatttggaaacacgatgaagtatatcaagatgtcagtgattgccaatctaggaagcataatttcactgctaattgcaacattgatatttggatttgagcctttgacaccaatgcagcttcttacacaaaacatcttgtataatcttggccaaattgcaataccatgggacaagatggaagattgttatgtgaaagtcccacagagatggtcacttaaaggtttagcaatgtttacattatggaatggacctctttgttctgcatctgatatagcaaccctgttattccttttgctatattacaaggtttcaagattagatttcgaattttttcgttctgcttggttcgttgaaggacttctaatgcaaacgcttatcatacacctgatacggacagagaaaatcccctttattcaggaagttgcgtcatggccagttgtttgtgctactattcttatatcatccattggcattgtaattccgtacacaacaattggaaagattctagggttcacagccttaccattgtcatacttcggatttttggttgtgctcttcttaggttatttttcgtttggacaaattatcaagaaaggctacattttggtattcaagacatggctt

SEQ ID NO: 59 - DNA, 26008 bp, artificial sequence
  Grape cv Pinot Noir PH1.genomic
ttataaaaaatatattttatttttattttatataattaaaatttttaacaaagaaaaatatcaaaaatataatcaagcttttwtctttttcaattwwwwattttawacattttaactaagaaaaaagttaaaatcacggtcaaattawatwccctwaataaaaagttaaatttgttaattttatattatttttttcttattattattattattattattttatgtttaaaatgttcataaataattaaaataaaaagatgaaatgaaaaaaatgaaattaaaaatcaaaagctaaaatgaagaaataagggggaaaaaaataacgtggcatgttggtttgtcacatgtcaagtttaaaaacattgataagtatgagtaataaaaaaataaaataaaattattataatgtttttgacttgtgatgtcaatttttttttcaaaacaagagaaaagtatggtagttggcggagtgatgaagagttaaaagggtaattttggaatttgttcatgttaatattcaagaacatgtttattaaattactttttaaaagttcaaaaaatctattttttgtttttagtgtacgtttagatagttctcccaaaaaaactttatggactggtacgaggaaaactataaccagcaatttattttatcattatttttatatcaatggatgtattttatttctaatggaatacaatatattaatttaattaatattaaatcaagcaatattaaatttgactcatggaattgaaggtgcataaaataagcccagtggaatctctggctgaataataagcccagtgggcctatcttctgtgtaaaactgtaaacccacccggggctgttgaaatccaggcctgacttagaagaaagctcagtatctagagttgggcctaaagtagccccatcaacaaaaaaatcatggcattgacgtgaatggtcacttcactgacatccatcatgggcagaacaatcttmtgaaggccggttcagtgtgattcctgtcattcaagtaaaacatgtttttccatatgtttcaatattattggtttaatgcagtaaagattgtgaaaaggtcggaaagcccaatcacagagctccaatcgaccgatgggtgtttagctttcttgcatatatgttcggaccttctgaatgcgactgtttcgtcttggtctcaaccatcaaccgggcaagttgtatccaaaacacagtacttgtttmccccaaaccaaaacaacaacagtagcattgtgggccgggcatcacgggtccagacaatgagacggcacatcatattttgttccggcctccgctcctcgttataccgatttatcatccaaaagcaaatttcacttcacttcatggtggacagaaggcacacaaaggaaaagagccagttgtgaagtggtatggggccaaatgcaaaagcggaacaccttccgaaatttcagtatgaagttggacacaaccccagtttggatgaacccatatttcttaatttcaataagattttttttcctccttttaaaaagagggaggtggcatataaaagagggccttgcaagaaaatcccatgaacatttcgattttaacttggttgggagcgagacacatttttgcttgggtcgtcaccctaatttataaagaaaaaaatggatagtgcaagttaaactatgattttgttggacactcctgatataaaggacgacttgatgaaaaaagtaagctgaaaagaataagaactccctcacttttcattctattttattggtctgggttatgctagaaaaatctgtgatggcttaggcgtaaagaggggagagagcacaaatctgcaattggtgtcgttttctggaagacacacatggaaaggaaaaaggcaaaggacatgagtggagaaaaccaggctataaagcattagaccaccctcactcyttttttcttttttggttatacatgattcttgccttaaagtcyycccagaaaattatatcaaaaagaaagaaaagaaagaaaagaaaacacggttaagtgtgtggaacgtgaatsttatcggcgccccaccaattcttattgaaagggagaaagccaaagraaaaaaacaragggttagtaacgtagatcgaccttkgcatatcatagtagatraacagttgtaaattggaattgtatggcgcacaatatcctatgatctaatgattagaagacaccatactagttgtttakgattgtacggtattttaattcaccccattttcctttttctagtctcaaccccaaaagcaaagttgatggaaataaaggacacttaataaattacatgaaaaatagtttttgragcaaaagaaggaatcaaatttgttgtaagatatgactcatattgagtgaaagayatgaaagaaaaatggcaaatgctggagtggagcggtgtgacgatatttatcattcaagtttgtatttttawtattgagggcrgacgataggttaggggtgyggaggggtgttgccyccgatatggttcagaggtatttttggaatttyattacctaakaattaatataaatataaycctaatttgaratatagtwaaagttttattcccacrttaaattcgtgtgttttttttttttttttcatttttctctaattttttcactagaaattgttgcaagaatatcaacaaaattaatgtttattaagcttttcggtgaaatatatgatgatataaataaatggggtgaagatacgagaatattaataaatgaaacttgagtataaastcaggaaactaaagggtgtatgaatgaatgttgtcggaataatgacgtattttgatacttattttgaaaaatcatctttgctcctgaagtagacgcaaatgaagttgggaaattgaagtgctaccataacctagaggctgatggatttttcatcatgccgagcatatgcggggtaccaggacaacccctttcatgttctctatataaaccctaagccatttccaacgacacattaagcctcccaacctttacaaaaggagcaccatgtcatatgatccaacagtgggttctaccaacattgtgaatggtgtcaccaccgtcgactgccaaaagcaagttcgttcatggaggcttctccgctctctyatggagctcctcattccaaggtgcaactgcatttctcttgaagaacaccgaattgaggaagaaaactatctccacagatacttctattcccaacccaccttcatttcctccaccgttgtcaccggcaccattttcgggtaccgccgaggaaaagttagcttttgtacccagacaaactccaagtccaccaacccaattctccttcttgaactggcagttcccacagccattcttgcaagggaaatgcagggtggaattctacgaatcackctcgaatccatagctgccaaaaatggcatggattcttacactctcttgtccataccagtgtggaccatgtgctgtaacgggaggaaagtaggctttgccgttaagcgcacaccctccaaggctgatatgaacgtgctagggctgatgggatccgtcattgtaggtgccggaattataagcgccaaggaactcaactgcgatgatgagctcatgtacctccgagccaattttgagagagttcgcagttcgtccaattctgagtccttccatttgatagaccccgatgggaacatcggtcaggagcttggtattttctttttccgctcaaggtgacctcaatcaagtctaccagaragcaacagcggcaacagttacacccttcgggtttttttgtgttgcgcccgcttctttggataggcaggactttggcttaattttttagcctcccattcatatattcctcttggcctttccagtttgctaattaattaatatgcttgagggagtgtcaacgcatcattatggccgattttggaggggaaggttcatcccataaccttgtttttccttcccttcccttccctttgagtgagcctaatcggccaattggtcatttygctatgtctctgtctgtcttgtggcttattggcatatgtatttggattgatttgaatgaagcatttaaatcctcttcttataatatctaatctagagagagagagacagttactattcataaatggttcttctggtgggtgggtggtgcccgtgactctggcctccataaattagctaacttctatatgggtgacatggaatataatatgtattaatatgtgataaattatcgagtcattggataaggttttaggttaacccagagacggttgtatctaacaaataggttgaccatggctcagcaacttgataaacccaccaaccccgaattaattcagatagttttgacttactgttacgtactggttaggccgtaggcttgtagctaccaaatcctatgacatcctttttttaatgcatgtatattttgtctctttggtgttttgatataaaaaagccaaacaatacaatggattttatggcatgtttttcctttacttcttcactttccaaggaacgaaaagagaaaacaagcaaataaaaaaattatggaaaaatcaatatgagaaaacaaaattagaatacaaaatttgatgaagtatttattttggagtcgaaaaagagaattagaatgaagttgattcctttttaaaccaactatttacttgttgtaaagaagaaaaattgaaatgtataattttaatatagtataataattttacatgaatgcatgataaatgaggaataataatgaaaatttgcaaaagttgttttaatttaaaaatgagaaattattttaaaaattttggaaaaattaaaattcaatgcataaatcatttcaattttaacttccatgytaggacataatcaaaatggtttaaattagaaaaatcaataaagtcrattcgattcaatrttttagtttctacttcatgaatcaaagtacatttattgagtaaaattcaaatttgatttccaactttgattgcaacaagaaattgaaattgttttgattttgcaatctagttaacaatagttaaaattagagtgaaacttttatttcattttcattatggtcttaaaaatcaaaattgacaaagatattgataatgaatgatgattagctagttttcacatagtttcyacttstttgccattgattgaacatttcgaaagattagattttatctctaatccaaaataaattttctcctcataagattatgaaacatacaatacacaaatataatcaatagatcgagattctaatttctactagaatttattatacacatattgaaaagcttcaaacaattatagcatcaacaatgcacaattgatctttaggcttcttaagcatcatcttatataaaaagaaatacgtaaaaaggtaaaaatgataaaaggatatatctcgatctatttactcactttaaaaacaaaaactttatttttattctatttcttatattgcaagtaaatacagaaaaatacttatttttttattttcctttttttcttatctaattatctctcatattattttcctttccgttgcgtcgttaaaagatgggaaacagtaaatgcatttacatcctaaaaatctatttgagaaaaggaaccacacaaaagatatttcaaatatatttgaaggttatattaaaatagaaaataaaaataagaaatcaatttcattctcattcattcatcgaaaaytttgaatttttttttttttttttggaataagaaaaaattattctgaaagcaaaaaacttattctttcattctttcattctatttcacattatgatttacctcgacttcatatatttcccttcctattaacatctaacacaccaagccaagtaaatatgtagtgatagatttagggcatttagtgagtgctgcttgttaaagatataggtggttggggctgcctttttgtgtgggtgtagtgtcgcatgagctggtgttgattccttgtgtcgtttatggacaccagggcgtgtgctacccatttgccttctggatratctatgttatttttaatttcttttcactctttttctaatttgtcatctaattctttttgtttctttgttttcatatttattccgcagactcgtacgtattcctttccaaacaaggtgcgtaaatccttattttggtaaattttattttgggagctattttaagtttgtacggagaaaaattgattaacgactaaataattaagagttcatttgagagtgattttagaaaatatttcaaatatttttaatatttgaatgataaaaattttcaagtattaaaaatattaaattttttttttaaaatcactattaaacaaactttaagaatgcgtttaataaagatttyatgatgcattttttatttttttannnnnnnnnnnntaaatttaagtattaaaaatattagaagtatttcctaaaattattataaaataaattctaaattctttataaaaaaaattattttatctatttaagtataaaattctttaattatattgatgttgtattttttaaatttaaaaatayttttamwaaaaatactttwaagtcaaacattgataaacacattcttaaaacatttttaataacaattctattaataataaattctttaatacttaaawttttttattaaaatrttatttttaaatatgaagaaaaactaaaracacttaacataatcacaaacaaactcacgatagcatgggacttcaaaaggattttgcccgactccagcaattcaccccgcagattatggggttctttggggggttttgtggtacatgaaccggctgagtttcaaatccaaaaactatcttgaacccggttcgggaktagcaatttgagaggtgggaactgggaagcacgatctgcaattcttcacaaactatacctaacggtcwtttgaaaggtggttagtgggaaggaaagacgtggagagtatgggaaagcgagagaaattcaacgagtccatgaaaaaagtaatttattttctatctaaaaccctctcttcctacctctctttcaaaccctttaaccccatgaatgcattctgtggttcatttcctctcttatctcggtgtcatagtagttctcaatcattaccgttgctattatggcaactcccagatttttcaatggaaattcccatcaaaactcctcatcttccaaccccattcgcgaacatcttgtgacgaggcctgatgatcgtaaacatggattcgccaattcggtttcagtttttttgcagcgattcatgtccggaagtaagtctcaaattctccatttttttaaaaacatttttggtttggtttttggagatgtgcttgatgtgggtctttcgtttttcttggaaatgcgtagagaaaatagatggaggatcacggacagaggaagaagagaaggtctactcttggttatatgcattggccaagtcggacaaggacttggtgtttgagtatgttcgatcgactgaaaggggtcagtgtataatctctttttcgtgtgattccattactttgggaatgtaattgttttggcttcagaaatttcgaatatcttacattaatggaagtatgattacgtgtttgatgaaatgtctaggagaagacagacagattaatttttggttgatctggcgtattagcgtataatataaattagtgagacaagaactccatttcaaaattttccccttttccatgatagcgtagaaagttgggggaaagtgattccttcaaaatttaattttctttccttatctttttcttgaacaacaaaagaaaactgtataatttttcctttccttctcttttcctatcaattttctttccgtcaaacgttttttgcaaactaaatataggttatgtttaagttttggaatgtactgaggaaaagggaaaaaaaaaatactaaagaaaatgatttcgtcatgtttggtttaccgtgaaaaatatgaacaacgaaaatcaaatatgattgaaatactttaaacctattttctatgttttaaaaataactttcatctttgtgtaattattttttaaaataactgttagaaaaaaattgaacaaaaaaaatgttatctgaaaatactttatttttgttttaagaatagaaaattgttgtttgttttctggttaccaaacgtgttttttttttttttttttttggagaataaaamtctgtttttgaaaatagttttcaaacaactcctaagattccttcgtggttttcttattcctaatactttctaagagccaaacaaagtattaatgaaatttcatttcctaatttctttcagcatacaataatctaaaacaatttattgagctcaaatctttgaaagaatagtagattgactattacttttgaaaaataaaaataggctaaaattcaataatttaccacacatcctatttctttcacctttcatgataggaaatgtcaagtgttgtatttacctccttaaattaagatgattttttttttctagtgaattgcggacaatcttgccaattaataaaataaaataaaatttgaactaagtttaaatgattataataaagatcttatagattaatgaaagaccttgagtacaaccttgttggtgctttatctattattaaataagtgggctagggtctatcttttagggttaaggagaggaataaatgatgaagtttatggttgcaaaaaataaaataaaaaaatatagaaaagaagagaagaagaaaaagagacaaaaacctagagtctcactaaataaatatttatagaactcttgatttttgttgtgtacatataagacatttatagaaccgataatctaaaattctatatataacatagatgaagcctargcatttaaataaaataatttggatttcttctcaatgaaatgcctaccatttaaaaaaaaagacaggaaataaaagaagaaatacatatgatgtaaatgcaagattcctatttggaatatgattttatctcatatgacatgaaatgggtatcaagtggtgaagggattttattggtcttgtaaaaagtttctccaactacacaagacttgatgagaaaatattagaagataaacatggcaattgataaagaggtcagtgtataaataagctattactaacatgaaatctctttaggactaccccaacttaggtcaagagtgagtgactttcttaacatccatatgcttgtttgttagtggaaaggagattgatttcggttttcaaactcataaaaaataaaaattatgcaaaaaacatgtttggtagcatgttctggaaaaaawttttgttaacagtttatgaaaatgagtcatccttagaaaaacgtagaaatattgttttctttgttaaaaagtwtgaacgctttaacaattggacatgatctaaaagaayaagtagttttttaacatgctcattattttcatttcccaaaatgagaatatttttccaaaaaccatttttattttcatcacttgagaggcactggaccttcctgtttgtaatggaaattgaattgaattttttttttcttcttygttcacatttacctagcatgtcattgtgttttgcaacaatcttacaattcagwgaacaaattgaccctttcagcctcaacctatgtacttgtttcaattatcagattactttactccctttgctttcatgcaggcctgagcttcaccgaagcagagaggagattgaaggaaaatggtccaaatgttcctgttgagtatcgtttcccctcctggtggcatcttctgtggactgctttctttcatccattcaatatcattctgatcgtcttgtcagcactctcatacttagccagtgacaatccaaatggatgcatcatgcttgtactggtttttataagtgtttccctccgattctaccaggtatatgttgctttatttgttctatcaaactggtatcacattttttcatttggcccttaatggttttcctgtgtcttccaggaatayggtagttcaaaagcagccatgaagctttcagaattagtaagatgcccggttaaagttcaaaggtgtgcaggtagagttgttcagactgaattaatagttcaagttgatcaaagagatattgttcctggggacattatcatttttgaacctggtgatctttttcctggtgatgttcggctattgacttcaaaacacctggttgtgaggtatgttacctggaacactwattcaatttatggctcaggattagtgagttctttcatgctatctattccttcctctacagccaattatagaacacgtgacttcctgcttcttgaacagccagtcctcactaacaggggagtctggagtaactgagaaaacagctgacatcaaagaagatcagagcactcctttgctagatttaaagaatatttgttttatggtaggtatwgacattgctagtccttctgtttgatacatatgatatagcttttgtattatattgacaatatttgagtaagcctcatatagaaaagtgaccattaggcaatgctaatggtatctgatgatttgggatcatttgaaaatttattgtgaataagttgggaaaattcacaatgcatgtcaacagaaaraaacacttatagtctttaacaactgacgatcataattctgtgaatttcacaaattattctggagttgtttgtatcatttaggrtatccatgatatattcataagtaattaaaattgggttttttttttttttttttccagtaagacctcccacattgtccatgacaacaaataacatgttggagatattttatggaggaaaatgtccaggttaaaatcatattactgaaaaagctccttccccttcagattttaaaatcatttacaaattattttcatgctttcacagtcttatgtggttaggctttcacctctttccttgaagcatttccaagacaacaacatcagtaattttctttatatgccattatcatgtcaaacacagatatatgattcattttttgtgattccatatttcagggaacgagtgtggtgtcaggttgtggaactggtctaattgtttcaactggatccaagacttacatgagcaccatgttttcaaatatagggaagcaaaagccaccggattactttgagaaaggagttcggcgtatatcttatgtgctgattgctgtcatgctcgtagtagtcactgccatagttttaacttgttattttacatcttatgatttgagtcaaagcattctttttggaatctcagttgcatgtgcacttacacctcagatgcttccactcatagtaaatacaagtcttgcgaaaggagcacttgctatggctagagatagatgtattgtcaaaagcttgactgccataagggatatgggatccatgtaagtttaaattgagctcatgaatgttctggcgcatatattacatcaatatataacaagttacctgccctgaattctctagctaattaacttctgggcaaatggagaagttcccagattttcaccatagattcaagttttaatcatacaattgaggcaaaactttgctttaaatttctcatattcactttttccttttaaccttcaatttttgttaaatcttcaagtaagcagaaactatgcactattcccttttccttcacacatatttaaaaatttttaagctaaacatttattctccttttccccctttttttgttatagggatatcctgtgcattgacaaaactggtacgcttaccatgaaccgtgcaatcatggttaatcatcttgacagttggggtttacccaaagaaaaggtcttgcgctttgctttccttaatgcttacttcaagactgaacagaagtatcctcttgatgatgcaattttggcatatgtatatacaaatggatatcggttccagccgtccaagtggaaaaagatagatgagattccttttgattttacgcggagaagagtatctgttatcttggaaacggagttgaatccaaaagaagattcctaccaatcactcgagaggtttgtggtaaccaaaggagcactagaagaaataataaacctttgttgttttattgatcatattgatcaggatgcaatcacaactttctccctagaagatcagcagaggattctaaatatgggggaggaattaagctatgagggattacgcgttataggagtggcagtaaagaggctacaaagggtatgtgacctatttcaactttcttatttcttatttttgtttttttctcatcttttctgtcttaggttctaattagctctatacattgttgttaaatcatttttcttcaaatagttaatttctttggaatttcattgcttacagaccagaagctgttcattagatgggttgtcaataggctaacatctttcccttcctgcacttactactgaccaagtctagaaatcaccttggtgtcaaccgtaacttgtataatttaaatttaatgtatatttgtagtcaatatcttgaagctagaaggggttaagcacaaaacagtttagtggaaaagttcatagactgacaacctctggctccacgtaatccaaaatctatgtatgggcatgtatataagcatctaaatatatttgatatatttacaattagcacttttgacatatttagctcagtgcttcatcatttgcttgaccttcatttgaatagaaaggttcttatagatcttagtttctgatcaatacacatggattatcttaragtgctgtattaactagagacataggagagtaagacagcttgcaagagttagagcaggagctgcctgagagttaagagcatggactttaaagaaccccacaatgccaaaatatacttgttcaatcatgtatttatgatagtatgttggtttgtttagataagttaaatcatcctcaaatatgagaaggattacagatgcaasaaaggtggattgttcatggttcaataatttgtaatctaaattcttgtcatgaagtttcagattctatatcccaacagccacttcctaaagatttttgaacarcttttggctaaatgtgctagagctcttgcatgttgtcacaggcacaaaattcttacaccagtttttgtgtctgtgcagtgcttagaagctctttaagccagcccacagaaggcatacgaaattacaaggcaccaaccctaacttgtatttttattaccttactgaataagaatacaagacataacttttagcaagactccygcaatctctttagtgagttcatgcttattattaactctctgctaggaggctctccagtcctccctttttatagagtggcgccactccttgttagaytaggagtacacaatcctagttctacttggactcttgttcataggtcatggcatcaaccactcctacttgaactagagatgagcaatcctactcctacccagagcgcaattacaataggaatctgaactcctactcatacttgatttgcaatccygttttgagtccaactcttcatgctgctgttgygtgcctctctctgcagctcgtgtgcatgcaagtccattcttagcctgcatgtctaggtcttccatgtcgaggcaacatgctcttgtatctatcatgttgctgtagtggcaccatgccctggccaagtcatctcttagtgcagctacgagtcatgccattgcacctatgrctcmtcatctctgtgcacaaggcattgcgcctttgtgtcaagccttaggcatcccatctgagtttgcatcactgctgctgcatcaatatgcctcrttcgagtccctcttggtgctgcaacatgccatggcatggcatccatggctcaccaagccgtctccttgcataaggcactgctcctctttgccaccttgtcatgtcatagttcactcatcaggccaaggtgctgccccatgaggccttggtgctgcatggtctgtgtcaaggggctaacatataccaggctcccaagtatatatgaaaaacaacttggtgccaaacctgctaacttgagatgagatagaaaccagtatagaaaattcattaacagttacattacgattttgttygtcttttgcagaaaacaagtgaaggaagcatagatagtgatgaggctaktgaatctgagatgattttccttggccttataaccttctttgacccacccaaggactcagcaaagcaagctctatggcgactggccgagaagggagtaaaagcgaaagtgttaacaggtgactcactgtccctagcagtaaaggtttgtcaggaagttggyatcagaaccacccatgtgattactggacccgatcttgagcttcttgatcaggatttgttccatgagaccgttaaaggggcaacagtactggctcgtctcacccccactcagaaactcagggtagtacagtccttgcagatggttggaaaccatgttgttgggttcctgggtgatggaataaatgactcacttgcattggacgctgccaatgttggtatatcagttgattctggagtctcagttgcaaaagactttgccgatattatattacttgaaaaggacctgaatgtacttgttgctggagttgagcggggtcggctcacctttgcaaacactatgaagtacataaaaatgtcagttattgccaatgtgggaagtgttctttcgatccttattgcaaccctgttccttcgatatgagccattgactcctaggcagctcatcactcagaacttcttgtataattttggccagatcgttattccttgggacaaggtggaagaagattatgtgaagaccccacagagcttttccaggaaaggcttacccatgttcattttgtggaatgcaccagtgtgcaccctctgtgacttagtcacgcttctgtttgtttacttctattatagagcctacactgcaaatgatgctagattcttccattcagcttggttcactgaagggcttctcatgcaaaccctaattatacatttgattcggactgagaaaattcccttcattcaagaggttgcctcctggcctgtgatctgttctactgtcattgtttctgccattggaatcgcaattcccttcacgccaattgggaaagtcatggactttgtccggctgccattttcatattatgggtttttggttgtacttttcattgggtatttttctgttggccaggtggttaagagaatctacattttgatctaccacaaatggctgtaaataacatattgaagtcatgagaagaaagttcgccagagattcaaaaacaggagcaatttttttcctgtgcatattatttagagtaaatgtaacamagcctaaattctctgaatgctttcttagatcattcacaattttcctctatcctttctgctctaacaacattacttgtatcacttgcaaatttgtrgaatatttgagtgtgatggttgaacaacaacaaagaaaggacatggatgagccacatttgtatctccctctttaactctagatcagtacgcaactttggttggatatatcataccatgtgatgcagatagagatagccacacactaatccaagtaacactgcastgttagacacttgcctgtttggtgaacttcctgcatgaaaatataagatggccatggtttattaagtaccattaaacaggaaagaaggtagccaaagaactgtattgacaactctatatgtgtgtgtgcatgtgcattttcaaatcaaatgaaggaaaagatgactgtaggcttttacccaattggagataattgttccgtatgttccattcaaaatcccagtgctttctatcatttttgagtgtccaataagcccatccaaaggaagctgcattataaacctctaactgggttcttccaaaattttgataatccatctgagtagcatttgcracattccattcgttcacccawtycccctgcaaccatgccattttcaagcatcaaaatttgtagtactacttgaattttctaatccacctctatataataagacttttatcaaatgcaaatcagtgttttaagttttggcaattatgactaggttcaaatgttagaactgaagtgtaacaaacttgaacaaaccagtttttgaatggaagaagacagcatggcaattatcttaccaataaaaaccagtggaccatttgctctgttcagagcccgtaattgagtttccctgctgttgtaaataaattggatattgtccaatgggttcatgttaacaaagaaattgtcgaagagattgtagtaatgtaaatctactaccaggttgtaagatcctatgtcagcctgaaaaagttccgayggatctgcaatgccaattctttggcaaactatcacataagcttctgaagaatactttcgaactatttgatagccttgcttgtaatatgaaactaagaggtccagtgaaactgaggcagcagatggttcatttaaaagctcaattcccagcagagtaggatgtttcccatatctgcaaaggtcaaattttagatcaccaccaaccagtacattaactaaggaattggttttgcttcagaagtcrcaagttgcaatgtccaaaggaaaaggattgagattgctcctagatgagtctgtttaaattcattcaacactcatgtcacagaaaataccataagagagagaatgcaagatgcagaaaatgaagagaatacttaaaaaacagttaaaagaaacatgctacacacacacacacacacacacacatatataatattaatgcctttgatggaaaagctacaattatagatgctagtagaagagggagctggagcaatgctgcaatggcaatgaagcagggtaactctaggagcagcataaacttcacttaatcttgtaggtctagcatgtgaatgaggatgtgtgtttgggatctgagaaaaaacaactgaacagttttttggagcaatttgatggagattyagtttggtgattgtttttattttttattttttatttctgtaaaacacttattgaaatcagccattttttgcatttcaatgatggaagatcattgatctaatagtgatatgttyaaatgccatggaatcagwagcatcatttckatttcatatggtatgcaaacaaaccataataacaatattaggctccccagctgcaagccaacaataccatcctaatcctaatccatgttggccaaggcaagtatgtaactgccctttagaagcaagaggatkccccatgttaatccaacactggcaggacacatgcaaattggcaaatttgtaacgagtaactggtatcttaaccctccaataactggtctagaatgtacctggaagctaaaaattctatcacatccaatgtttgtgaaatgtaacttgcagatgtaggccagccagatgaaccatctctactagcactatgttccatcccattctgggagccaggagccgcatgcaggtcaattatgcaccttatattataggctctgtgcaacagtttttcagatacatatcaaaaggatgcaaatttaaagtcccaacagtgaaaacagaaacaggttcaaaacaagacctactgcgcccatgagaatgcattatccagagcttccaaagttcccccaataaaaggagccggtggattaggatcaaaagcaatccaccaaccaacaggaatcctcacagtatttattccatgtctatacagaaaaatgaaatcttctatagtgataaagctgtttctatgtctctgcagttttcaaaaggccaatacatatcaaataaacaagatactgagattcatggagaaacctgttctacaatctagcaaaaatagctcaayttctgaaaatcacaaagtgatatccaaaatattatrctaactggctaagatatatggttcatgcttrgcatatagttcaatcaacaaatcatgatgacaatattcacaagtgggttaaaataagaacattggaggatgccaaaactatatcacaaagtaaagctcatataccaactatgtgtggacacaggtttggggatggacacaatactracaagccaggagttttttgttgcttaaagtgcccccaaagaaacctttctgcagaaaattaaaaatgttttgcaaacatacaagtaaaattagttggggaaaaatactttgttgagtttgagttgtagatacaatattacatttaagcatcacctcttggcaattgtgttttttttataggtaaatttttgttcctattgggacttgaacctggaacctccaacaaacctcccatcctttactgcttgagctaggcctcaagggcatcgcctcttagcaattgttgatactcaatagagttagatatcaaaacattacatcatattaaattatacagcataaaacaaaggcaaaacatgctctgggctccagattccttaacactatattgggttgtttttttcttagccaaacctgttatatcctaaagtccataacccataaattatgcaatatgagttcatccaataaattctgttaaaggtgctatccaatataattggtcaagaagtttgagaaaaaaaaaagaaaaaataaataaaagaagtcaaacaggaaatttggagctaccttaagaacttctttggctttatcatgtccatacccatttgcaagctggtaatcaccatgtatgttatttgctacaattgtcatttcaaatgtggctgcattatcatcccatcctggcatccctggatagtctgctgagagctgattagctagtgtagcctagacaagtgaagtataagactatcatgttttagtattagaaaataaaagaggggaccttgaagagatattaagaaaatgtcatctaggaggatacttggtaagtttggtaatggaatagtgggtgggtgactgggtattccaacaagcatttataccagtatttgatgacagcaagctaaacaaaaactcaaacaatctgagattgtaaccaacttaattacttgcataacattgaacaactggattaagttttttaattttccctcaactattcctaaactattcctgaaattctagaataatcctgaaattattactgactactcaacttccagcactattcttaaataattcacaaaggcagataggtgccaaaatctgctggtcttgctgtgggcaatcagagmtattgacaatggaaccatagagaagccaaaacaatggatgactgaaaatgttcaacctggagctctargaatggtagtcatgccacactgcaygcatgacaagtttgccattacaccattmttgactgtctacccaactaaagtgagtaaattaaattctgaaatagtaaagtaagcgattgaatacacctgcagatagttcccattcttcagtttgatgtgaactctgttgtcataatttctttcaacataaaatgtttcctttattgacgatgatccygccattgcagagacagagcctccctcaccatcgcatgytaaaaactgcccttgagaagtgcggaactgaaactctgaatcagaaaccctccataactgcaggacactaatgaaataattaatttccatacatcacaagaatttgtaatatcaaacagcctacctttgattcattagattatgacaccctgtttggtgcacctaatgagataactgtatttgtttaatagatacaagtctaaaagttaatccttagaataaatgcagttattatcatcatcctgcatgtgcttcaggcaaagacaacaaggtctgaacttaagaactagattaataacaatgattccaaccaagacaccactaacacattgaataaccagcacaatgcacaattaactgagaacctagaaattctacgattcaatttctaaataagcattctacactaatttctggaaataacagtctcttgatttaatatccaactccccctccaggaagcaaggaaaatggaagggaaaaaagaagggaaaaagaacacaagctgatgtttgaaaatatatcataagcaattttgtttcacaagaaaacaagtttatttcatcctttttataagcagcaccttcataaaatcaccaataactaaatttttttctatttaagtcatgatgagcaaacactggagtatttcatataacttaagagtagaatggtgaatttatccaaattctaagatccgaataactggtacaaggcattgctaagtggttttgagttgagaaattgttgtatgtcccattaacaaagcgcatcatccaacttaaggttgttgaccatgtgaaaaatgaagtcaaaagcacttcaatgctaatttgccttagtttatgatagtgtgtattatctagcagaggaacaagtctcagacagatacagccttaggacagaaatttctcatggtagttttcttcctttccttttcattwactagatctaaacacagcaatttcaggtttgcttagcattggtaagaatgctgccactagatctaaactgaacacaataccacatgtttccttgcattattaaatgatcactcacatgcctcacatgactcaaccacctaatgaactttgaatacagaaatcagaaataatatattcaacatcctcatgaataatgcttgagaagagtgcagcacactctttggatcatttcgatgtgcaaactgttgctcgatcttttgacatcacttcaatttttttgctttgaaagaaggccataaaattctaaatatggaccacaacagyagaactcraattttgctaacctcaaccaagttgtgcttgtgtttmttacctcattgctttacttttcgagtcaaactcaaccatgtaataaaaggagtttaaragtgttttcccagggtagaggctagaattcaagcataatcttttaataccacaagttaaaagttctaatttaccacatatcttyaatttctaaaagtgcctatcaatatgtatattcaatttctagaagtagttgcatagaccttctcttcccccaacaatatatatatatatatatatatatatatatatatatatatatatatattgctgctggaagcttawaatttcaatttcatgttgcagtaacaggctggcaaagaaatgtgtgaagttgaactaattctgttgtaagaatgcatgttaaagtgttcaatcaactccattagcagttaattcatgttggaatggatgaatttctttacctccttaaaattgcagctacacacctacaccttgaattaaaatgactggccaaagagttaaaactacgagaactagtcaaaaagattatcataaattttttggtgaacaggaaaaaaaaatttaaatgaaaatatctgattatatgatcaaatctactaatttaaagaaaccacttatttatgacataaagtatcttacacagtaacatataaaaatcaagagaaaaaaattaagacaagcgcataacaataggtcagacatctaaccctgaaggtttcccatgaagaaggaacatctttgtctactgtaacacccatgcctcctccattctctgcagatacatacttctgcaacatcagtgacttgaattgaacctctgttccatcctaaatttccatagccagaaaagggttagccagatcagtmacctaaatcataaaagaatgccaagccaatgaagcagcacaattacgataaaggaaagcttacgagcatgtctccatttggaatgccatcaaacaatgaaggtttaatccagccttccacaaccagccaccctcccaggttcactcctctaactttttcacccccttgtactaagtccacttgaatcaaacaatagaaaatgttttataatttgcaaattgggttctgttttccctttttttttttcatcctccaaatacacagcatttcaagcaacaatctaaaatcaagaaaaaggcgacaagcaaratagaaatgcaggtgcccattcygataaaaaaayaatgggttcttgttcttgtcgttcccayactcgaatacattgcatttccaagaaccaaacgaaaaaaaaaaaatcaagaaaagggagttatagaatttacccgagtatgagaaaataagccgacaacagaggagaaatgcaaatacccatttgcggaaaacgagttccatgtagaatgcaggcggcggaaacatcaagaaggcgcaaatatagagtccatctacaacaaaagaacaacycaaataaagaaactgagattgtttgaaaacccacttgggcgaatcttagtaattctttccttatcactcaaattattccaaacataaacaattaaacaagcatmaatatttagagytagcctctgcgattattrcatcattaattaaattgagaaacccaagtgtagastcgagacaagctaaaacccattaaaataagagcagaaggaaagaacaatcttatttttattccaacaatgctttaaaaaaagggtggaaagttttcacgtgtacggcatagaatgtggattgaaaagggaaggaaagggcgactttgagtgaagatttgggatgctgggtcttgattggttggatgaagaaaaggttagaaaggttagagaacgtgcctttaagtaaagaaattgggaaagtattcgagatgacaatggttacagtgcaactccattacaccttatcttcacgttcgatcttccggctcccaccaccttcctttgctaaatctgggttcagaggaaacggtctacgactttctttttctttttctaatttcttcttccttctattatttcctctcttttcttttctcttcttttcttctattttcccttcatacatttaagaaaagtataggttgaagatcactgataatcaaattatttaatcaacatgttttgatcttcagctaactcatgaagatcttccttcgtctttgagagaaaaataatatacccattaaatcaaagggygtagcatcwttatatttgmaccagtaaagccaaaagtcttasaagaataccagctgctcgttggagacaaagcaasttagaggtacatttcaacagtctgataaattcattttgagaawtttttrcaggccgctcagtatgggccttaaatgtttcctaaaccaaaagcctarcccaaaatgaagacctttcccaacgatataagagacagagacagagccagaggatttattgtgagacaagtacaacaatatagcctgggccctaggcttgaccttgagcttgagcccactttacttctaacaggctctcaaagcacaggatcaagcgtgaggcactttgagagaatgccyactcatcttctttccttgggtgacttcgcactttgcatacgttttgggggctctttgtgcacactcaactcttttctatacctataaacatgacttttattccaataaaattgtactagcacaaaaaattattgtataaggacaaktttcacaagtttggcttactattggactttcaaggccaaataagttaacaagagacttttattgaattaagtgagaaattataccgggaatattatataatttgacaaaacccctttaacaagggaagtcccccaaaagcaatgtagtaaatattattttatagtactctcatttttgtctttcaattatggaacttcacaatagctattagagtgtttgacactgaggctgatatattagtaactctccgttgatgttgatgtatgcagtgcatggtgccacgctagtggttttacttttacatacctaatatgatatgacccatattagattggcatcctacatgattacttgagcaaggatcctatattgaggatcaaaatattaattaaacttttttcccaccgttgaattaggattagatactacttacttcttcgattcgttgatttaaactcaacaaatgggaatttgttataaagtattactccattgatgccaccattattgatatggtaatacctaccccaattaaagcgatagaaagctattgtgattgacattgaaaatataattgtatttttatgatgagatgtgtatgactttttttattttttttatatccaattcttctgacatcaactatttgatttgatggcgtacatcatatcttctaattctactcttatatcttgttctcctttgatatgacatttttattttctataaaagcaccattctcatcatacaatttataattatcatcatatggtctaaaatttttcatttgttactttcctctctagtccacatgtgaccaaccaaaatattgcctcctaacataccattttatgaatttaacagagatataataaatatatatattaaaatttttaaaatttttattaacttgatttatgataaagttgtttgataaagaaaagaagtatatttattagttatgatgtaatttttacattgaaaatatctttgaaaggaaaaactttatatgaaaattaatttaaaatttatattttctcatggcctaagggttataactttataccataaaataaatagtaacttttcctccacaaaattttatttattttataattttataaaataatttcaaaaattatataattgttattattattattattttttattcaagtggcctttgyatgtggtggtgccctattggtttaaagaggtgggtatgatatccaaaagygattttcggtacccgtagtatcattttttgatatcctagcataattcgatatgagaattaatwactctcatttaaaaaaagaaaggaaaaaatacttatcaacaaagatgtaatttttacattgaaaatgtctttgaaaggaaaaactttaatataaaaattcagttttcaaataattacttaaaattataaatarataawtttaatataaatttaagatccaacaaaaataattaaaatataatcttttgaatattgaactttaatataaatgatacttcaatattaatttatatattacaaagttwctatttaaaaaacaacttcaatataaattaattcactaaaacttgaaattaaatagaagcaggtgtcatatgaggaaataaatttacttaaaaatcaaaagtgaaattaatttttaaatattttttttttcaaaatatacaaacggcttaaatttgtcaaatccaattgacaattttccaagtttggcttagtattggactttcaagggcaaataagttaacaagagacttttattgaatcaagtaagagaaattataccgggaatattatgtaacattgattagactatactgattgacaaaatccctttaacacgggaagtcccccatatgcactgcatggtgccacgatagtggttttatgcacctaacatgatatgacccatattagaggaagcctccctacatgactacttgagcaaggatcctatattgaggatcaaaatattaattaaatttttttcccaccgagggataggaatttgttataaagtattactccattgatgccaccattattgacatggtactacctaccccaattaataaaaagctattgtgattgacaatgaaaatataattttatgtttttgatgagatgtctatgtcttttttttatatattttttttatttttatatctaacttttttgacattaaccatcttatcattaatttgacttaatttgatggacgtatatcatatcttctaattctactcttatatcttattctccttcgaccatgacatttttattttctataaaagacatttaattctcatcatacaacttataatcaccgtcatatgatctaaattttttctat